(19) United States
(12) Patent Application Publication
(10) Pub. No.: US 2022/0081666 A1
(43) Pub. Date: Mar. 17, 2022

(54) METHODS OF CELL CULTURE CLARIFICATION

(71) Applicant: ICHNOS SCIENCES SA, La Chaux-de-Fonds (CH)

(72) Inventors: Patrick VETSCH, La Chaux-de-Fonds (CH); Romain METTE, La Chaux-de-Fonds (CH)

(21) Appl. No.: 17/415,651

(22) PCT Filed: Dec. 20, 2019
(86) PCT No.: PCT/EP2019/025496

§ 371 Date: Jun. 17, 2021

(30) Foreign Application Priority Data
Dec. 21, 2018 (EP) 18020660.9

(51) Int. Cl. C12M 1/00; C12M 1/26; C12M 1/33

(52) U.S. Cl. CPC C12M 29/04; C12M 33/08; C12M 45/02; C12M 47/02; C12M 47/10; C12M 33/10

(57) ABSTRACT

The present invention relates to methods of cell culture clarification. In particular the present invention discloses methods of cell culture clarification useful in the manufacturing process of biopharmaceutical molecules, such as antibodies.

### TECHNICAL FIELD

The present invention relates to methods of cell culture clarification. In particular the present invention discloses methods of cell culture clarification useful in the manufacturing process of biopharmaceutical molecules, such as antibodies.

### BACKGROUND

The manufacturing process of a biopharmaceutical molecule, such as an antibody, is complex and it comprises different steps, each requiring extensive optimizations. The process begins with the selection of a cell line to use for expressing and secreting the biomolecule of interest. Once selected, this cell line is amplified and cultivated at larger scales in bioreactors where the biomolecule of interest is produced under controlled conditions. These steps are referred to as the upstream process (USP) part of the whole biomolecule manufacturing process.

In order to remove residual biomass and impurities from the culture medium containing the biomolecule of interest, a clarification process is necessary. This process is the first step of the biomolecule purification and it is usually considered to be at the interface between the USP and the purification steps (referred to as downstream process (DSP)). Cell culture clarification requires multiple steps directed to the removal of different types of impurities spanning a wide range of size, including residual cells, cell debris and eventually DNA and host cell proteins (HCPs). Importantly, the clarification process must meet the downstream process requirements in terms of product stability and purity and at the same time it needs to be robust enough to accommodate eventual variability in the cell culture.

A challenge of the clarification step is to result in a clarified cell culture with low turbidity so as to be compatible with the subsequent purification process; e.g. a low quality clarified harvest could cause a rapid attrition of the purification columns due to the presence of impurities. This may impact the lifetime of the purification resin as well as the overall process design and finally its yield and the cost of goods. Given the importance of the cell culture clarification, it is essential to have a high understanding of the process and its tight control; moreover it is crucial the optimization of the cell clarification step in order to efficiently reduce the turbidity of a cell culture and to improve the balance between the quality of the manufactured biomolecule, the yield, the time and costs of the clarification process and its robustness also in relation with the other USP and DSP steps.

### SUMMARY

The present invention relates to methods of cell culture clarification. In the manufacturing process of a biomolecule of interest, such as a therapeutic antibody, the cell culture clarification is the step which allows the removal of cell culture material such as cells, cell debris and impurities from the cell culture medium to obtain a clarified cell culture where mainly the biomolecule of interest is present in the cell culture medium. Importantly, the higher the turbidity of the initial cell culture is, the more difficult it is to efficiently clarify the cell culture. Given that the manufacturing of biomolecules in bioreactors has nowadays reached an elevated productivity, which implies elevated cell concentrations in the culture, biomass accumulation levels have challenged the clarification process. Therefore, the ability of the clarification system to reduce the turbidity of a cell culture at high throughputs is fundamental, especially when the cells are cultured with high density.

In particular the present invention relates to a method for clarifying a cell culture comprising a cell culture including a biomolecule of interest and having a turbidity between about 1000 NTU and about 6000 NTU characterized by comprising (a) a primary clarification step which removes cell culture material of size equal to or greater than about 0.2 μm and (b) a secondary clarification step comprising filtration which removes cell culture material of size equal to or less than 4 μm, wherein said secondary clarification step has a maximum throughput equal to or greater than about 80 L/m2 and wherein said primary and secondary clarification steps lead to a turbidity reduction equal to or greater than about 90%.

More specifically according to the present invention, the primary clarification is selected from the group comprising depth filtration, centrifugation and flocculation.

In one embodiment, the primary clarification is depth filtration performed by a first depth filter with exclusion range comprised between about 0.25 μm and about 30 μm. In more specific embodiment, the first depth filter has exclusion range comprised between about 5 μm and about 30 μm, or comprised between about 0.5 μm and about 10 μm.

In another embodiment of the primary clarification is centrifugation performed at a relative centrifugal field comprised between about 500 G and about 3000 G for a time comprised between 1 and 10 minutes.

In a further embodiment the primary clarification is flocculation performed with a flocculation agent selected from the group comprising calcium phosphate, caprylic acid, divalent cations or positively charged polymers like polyamine, chitosan or polydiallyldimethylammonium chloride and which is added to the cell culture at a percentage of about 0.03% v/v.

According to the method of the present invention, the secondary clarification is performed by a second depth filter with an exclusion range equal to or less than about 3.5 μm.

In one aspect of the present invention, when the cell culture has a turbidity less than about 3000 NTU, the maximum throughput is equal to or greater than about 250 L/m2 and the turbidity reduction is equal to or greater than about 99%.

In another aspect when the cell culture has a turbidity equal to or greater than about 3000 NTU, the maximum throughput is equal to or greater than about 80 L/m2 and the turbidity reduction is equal to or greater than about 99%. More in particular, according to one aspect, when primary clarification is performed with a depth filter by an exclusion range comprised between about 5 μm and about 30 μm, or comprised between about 0.5 μm and about 10 μm, and the secondary clarification is performed by a second depth filter with an exclusion range equal to or less than about 3.5 μm, and when the cell culture has a turbidity equal to or greater than about 3000 NTU, the maximum throughput is equal to or greater than about 80 L/m2 and the turbidity reduction is equal to or greater than about 99%.

In a further aspect, when the turbidity of said cell culture is equal to or less than 3000 NTU, the primary clarification step and the secondary clarification step are performed by a single filter with an exclusion range comprised between about 1 μm and about 20 μm, the maximum throughput is equal to or greater than 110 L/m2 and the turbidity reduction is equal to or greater than about 98%.

In certain embodiments of the present invention, the primary clarification is preceded by a cell culture pretreatment step of acoustic wave separation.

In one aspect, the method of the present invention also comprises (c) a bioburden reduction step performed by one or more sterile filters with exclusion range equal to or less than 0.5 μm to obtain a clarified cell culture comprising the biomolecule of interest.

In another aspect, the method of the present invention comprises a further filtration step subsequent to the secondary clarification performed by a membrane absorber with exclusion range equal to less than 0.2 μm.

In another further aspect, the method of the present invention comprises a further a step of (d) subjecting the clarified cell culture to one or more steps of purification of said biomolecule of interest.

According to an aspect of the present invention, the cells are mammalian cells.

According to another aspect of the present invention, the biomolecule of interest is an antibody or an antibody fragment thereof.

The current invention also discloses a cell culture subjected to the method of anyone of the preceding claims.

The current invention also discloses a process of production of a drug substance comprising the steps of:

(i) seeding cells expressing a biomolecule of interest in a cell culture medium;
(ii) culturing said cells for a period comprised between 10 and 18 days, preferably for 14 days;
(iii) subjecting the obtained cell culture to the clarification method according to the present invention;
(iv) add excipients to the purified biomolecule of interest.

Unless otherwise defined, all terms used in this disclosure, including technical and scientific terms, have the meaning as commonly understood by a person skilled in the art to which this disclosure belongs. By means of further guidance, term definitions are included to better appreciate the teaching of the present disclosure.

As used herein, the following terms have the following meanings: “a”, “an”, and “the” as used herein refers to both singular and plural unless the context clearly dictates otherwise.

The term “biomolecule of interest” as used herein refers to a product of interest, which is desired to be purified or separated from one or more undesirable entities, e.g., one or more impurities, which may be present in a sample containing the product of interest. In a preferred embodiment of the present invention, the biomolecule of interest is a polypeptide. In a more preferred embodiment, the biomolecule of interest is a protein. In an even more preferred embodiment, the biomolecule of interest is an antibody or an antibody fragment thereof. In a particularly preferred embodiment of the present invention, the biomolecule of interest is a monoclonal antibody or a monoclonal antibody fragment thereof.

In the present invention, the term “antibody” and the term “immunoglobulin” are used interchangeably. The term “antibody” as referred to herein, includes the full-length antibody and antibody fragments. Antibodies are glycoproteins produced by plasma cells that play a role in the immune response by recognizing and inactivating antigen molecules. In mammals, five classes of immunoglobulins are produced: IgM, IgD, IgG, IgA and IgE. In the native form, immunoglobulins exist as one or more copies of a Y-shaped unit composed of four polypeptide chains: two identical heavy (H) chains and two identical light (L) chains. Covalent disulfide bonds and non-covalent interactions allow inter-chain connections; particularly heavy chains are linked to each other, while each light chain pairs with a heavy chain. Both heavy chain and light chain comprise an N-terminal variable (V) region and a C-terminal constant (C) region. In the heavy chain, the variable region is composed of one variable domain (VH), and the constant region is composed of three or four constant domains (CH1, CH2, CH3 and CH4), depending on the antibody class; while the light chain comprises a variable domain (VL) and a single constant domain (CL). The variable regions contain three regions of hypervariability, termed complementarity determining regions (CDRs). These form the antigen binding site and confer specificity to the antibody. CDRs are situated between four more conserved regions, termed framework regions (FRs) that define the position of the CDRs. Antigen binding is facilitated by flexibility of the domains position; for instance, immunoglobulin containing three constant heavy domains present a spacer between CH1 and CH2, called “hinge region” that allows movement for the interaction with the target. Starting from an antibody in its intact, native form, enzymatic digestion can lead to the generation of antibody fragments. For example, the incubation of an IgG with the endopeptidase papain, leads to the disruption of peptide bonds in the hinge region and to the consequent production of three fragments: two antibody binding (Fab) fragments, each capable of antigen binding, and a cristallizable fragment (Fc). Digestion by pepsin instead yields one large fragment, F(ab′)2, composed by two Fab units linked by disulfide bonds, and many small fragments resulting from the degradation of the Fc region.

The term “antibody fragments” as used herein, includes one or more portion(s) of a full-length antibody. Non limiting examples of antibody fragments include: (i) the fragment crystallizable (Fc) composed by two constant heavy chain fragments which consist of CH2 and CH3 domains, in IgA, IgD and IgG, and of CH2, CH3 and CH4 domains, in IgE and IgM, and which are paired by disulfide bonds and non-covalent interactions; (ii) the fragment antigen binding (Fab), consisting of VL, CL and VH, CH1 connected by disulfide bonds; (iii) Fab′, consisting of VL, CL and VH, CH1 connected by disulfide bonds, and of one or more cysteine residues from the hinge region; (iv) Fab′-SH, which is a Fab′ fragment in which the cysteine residues contain a free sulfhydryl group; (v) F(ab′)2 consisting of two Fab fragments connected at the hinge region by a disulfides bond; (vi) the variable fragments (Fv), consisting of VL and VH chains, paired together by non-covalent interactions; (vii) the single chain variable fragments (scFv), consisting of VL and VH chains paired together by a linker; (ix) the bispecific single chain Fv dimers, (x) the scFv-Fc fragment; (xi) a Fd fragment consisting of the VH and CH1 domains; (xii) the single domain antibody, dAb, consisting of a VH domain or a VL domain; (xiii) diabodies, consisting of two scFv fragments in which VH and VL domains are connected by a short peptide that prevent their pairing in the same chain and allows the non-covalent dimerization of the two scFvs; (xiv) the trivalent 10 triabodies, where three scFv, with VH and VL domains connected by a short peptide, form a trimer. (xv) half-IgG, comprising a single heavy chain and a single variable chain.

Depending on their nature, antibodies and antibody fragments can be monomeric or multimeric, monovalent or multivalent, monospecific or multispecific.

The term “monospecific antibody” as used herein, refers to any antibody or fragment having one or more binding sites, all binding the same epitope.

The term “multispecific antibody” as used herein, refers to any antibody or fragment having more than one binding site that can bind different epitopes of the same antigen, or different antigens. A non-limiting example of multispecific antibodies are bispecific antibody, which have two binding sites that can bind two different epitopes of the same antigen, or two different antigens.

The term “monoclonal antibody” (MAb), as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The biomolecule of interest, such as an antibody, can be produced by introducing genetic material encoding said biomolecule of interest in host cells. The term “host cells” refers to all the cells in which the biomolecule of interest codified by the artificially introduced genetic material is expressed, including those cells in which the foreign nucleic acid is directly introduced and their progeny. In the host cells it can be introduced an expression vectors (constructs), such as plasmids and the like, encoding the biomolecule of interest e.g., via transformation, transfection, infection, or injection. Such expression vectors normally contain the necessary elements for the transcription and translation of the sequence encoding the biomolecule of interest. Methods which are well known to and practiced by those skilled in the art can be used to construct expression vectors containing sequences encoding the protein of interest, as well as the appropriate transcriptional and translational control elements. These methods include in vitro recombinant DNA techniques, synthetic techniques, and in vivo genetic recombination. Cell lines suitable as host cells include and are not limited to bacteria, mammalian, insect, plant and yeast cells. Cell lines often used for the expression and production of therapeutic antibodies include mammalian cells lines such as Chinese hamster ovary (CHO) cells, NSO mouse myeloma cells, human cervical carcinoma (HeLa) cells and human embryonic kidney (HEK) cells.

In a particular embodiment of the present invention the cultured cells are mammalian cells, more in particular, they are CHO cells.

The terms “cell culture” and “culture” as used herein are interchangeable and refer to the growth and/or propagation and/or maintenance of cells in controlled artificial conditions, and they indicate a cell culture which comprises a cell culture medium and cell culture material comprising cells, cell debris, for instance generated upon cell death, colloidal particles, such as DNA, RNA and host cell proteins (HCP), and (bio)molecules secreted by the cultured cells, such as the biomolecule of interest.

The cells of a cell culture can be cultured in suspension or attached to a solid substrate, in containers comprising a cell culture medium. For example a cell culture can be grown in tubes, spin tubes, flasks, bags, roller bottles, bioreactors. In certain embodiments of the present invention, at the end of the cell culture the obtained titer of the biomolecule of interest is below 10 g/L, in other embodiments the obtained titer of the biomolecule of interest is comprised between about 1 g/L and about 10 g/L. Non limiting examples of the obtained titer of the biomolecule of interest include: about 0.1 g/L, about 0.5 g/L, about 1 g/L, about 1.5 g/L, about 2 g/L, about 2.5 g/L, about 3 g/L, about 3.5 g/L, about 4 g/L, about 4.5 g/L, about 5 g/L, about 5.5 g/L, about 6 g/L, about 6.5 g/L, about 7 g/L, about 7.5 g/L, about 8 g/L, about 8.5 g/L, about 9 g/L, about 9.5 g/L, about 10 g/L.

The cell culture to be clarified is also referred herein as cell culture fluid (CCF).

When the production of the biomolecule of interest has a commercial purpose, often the host cells are cultured in bioreactors, under conditions that aid their growth and the expression of said biomolecule of interest. The term “bioreactor,” as used herein, refers to any manufactured or engineered device or system that supports a biologically active environment. Optimal culturing conditions are obtained by the control and adjustment of several parameters including: the formulation of the cell culture medium, the bioreactor operating parameters, the nutrient supply modality and the culturing time period. The formulation of the culturing medium has to be optimized to favorite cell vitality and reproduction; examples of constituents of the cell culture medium include but are not limited to essential amino acids, salts, glucose, growth factors and antibiotics. Important bioreactor operating parameters include: initial cell seeding density, temperature, pH, agitation speed, oxygenation and carbon dioxide levels. Nutrients can be supplied in different ways: in the batch mode culture all the necessary nutrients are present in the initial base medium and are used till exhausted while wastes accumulate; in the fed-batch culture additional feed medium is supplied to prevent nutrient depletion and prolong the culture; differently, in the perfusion modality, cells in culture are continuously supplemented with fresh medium containing nutrients that flows in the bioreactor removing cell wastes. The culturing period is important as it needs to be long enough to let the cells produce a consistent amount of product but it cannot be too long to impair their viability. Commonly used bioreactors are typically cylindrical, ranging in size from liters to cubic meters, and are often made of stainless steel. In the embodiments described herein, a bioreactor is made of plastic or of stainless steel. It is contemplated that the total volume of a bioreactor may be any volume ranging from 100 mL to up to 20000 Liters or more, depending on a particular process. Non limiting examples of bioreactor volumes include about 100 mL, about 200 mL, about 500 mL, about 800 mL, about 1 L, about 5 L, about 10 L, about 20 L, about 30 L, about 40 L, about 50 L, about 60 L, about 70 L, about 80 L, about 90 L, about 100 L, about 200 L, about 300 L, about 400 L, about 500 L, about 600 L, about 700 L, about 800 L, about 900 L, about 1000 L, about 2000 L, about 3000 L, about 4000 L, about 5000 L, about 6000 L, about 7000 L, about 8000 L, about 9000 L, about 10000 L, about 15000 L, about 20000 L.

Bioreactors useful for present inventions include but are not limited to small scale bioreactors, single use bioreactors (SUB), shake flask vessels, large scale bioreactors, batch bioreactors, fed-batch bioreactors. In a particular embodiment of this invention cells are cultured in 3 to 5 L, or 50 L SUBs in fed-batch mode for a number of days comprised between 10 and 20 days, preferably between 12 and 18 days, most preferably for at least 13 days, even more preferably for 14 days in a cell culture medium.

The terms “cell culture medium,” and “culture medium” and “medium” are used interchangeably herein and they refer to a nutrient solution used for growing cells, such as animal cells, e.g., mammalian cells. Such a nutrient solution generally includes various factors necessary for cell attachment, growth, and maintenance of the cellular environment. For example, a typical nutrient solution may include a basal media formulation, various supplements depending on the cell type and, occasionally, antibiotics. During cell culture the cell culture medium may also contain cell culture material such as cell waste products, host cell proteins (HCP) and material from lysed cells. The composition of the culture medium may vary in time during the course of the culturing of cells.

The terms “clarify”, “clarification”, “clarification step”, “clarification process” as used herein are interchangeable and generally they refer to one or more steps that aid the removal of a part of the cell culture material from the cell culture, such as removal of cells, cell debris and colloidal particles, to obtained clarified cell culture, also called clarified cell culture fluid (CCCF) herein, comprising the biomolecule of interest.

The efficiency of the clarification step is crucial to facilitate the subsequent downstream processing steps of purification of the biomolecule of interest. Characteristics of the cell culture that have an impact on the clarification step include the total cell concentration, the cell viability, the initial turbidity of the cell culture to clarify, the concentration of biomolecule produced by the cultured cells. The term “Total cell concentration” (TCC) refers to the number of cells in a given volume of culture. The terms “Viable cell concentration” (VCC) refers to the number of live cells in a given volume of culture, as determined by standard viability assays (such as trypan blue dye exclusion method). The percentage of living cells is called “viability”. In general terms, a higher TCC implies higher biomass to be removed from the cell culture and therefore higher impact on the clarification. Similarly for the lower viability, given the higher presence of cell debris. The term “turbidity” refers to the cloudiness or haziness of a liquid caused by large numbers of individual particles. In particular, the turbidity indicates the amount of material and small particles inside a liquid capable of light diffusion. More in particular, the turbidity of a cell culture may be due to the presence of cells, cell debris, colloidal particles, such as DNA, RNA and host cell proteins (HCP), and of the biomolecule of interest. The aim of the clarification step may be considered the reduction of the initial turbidity of the cell culture to a lower turbidity of the clarified cell culture to obtain a clarified cell culture with the highest concentration of the biomolecule of interest and smaller presence of other cell culture material. In one embodiment of the present invention, a threshold has been set for the maximum the turbidity of the clarified cell culture, also referred inhere as CCCF. In particular the turbidity of the CCCF has been set equal to or less than 10 NTU. Nevertheless, at a turbidity above 10 NTU, clarification could still be efficient but the risk of clogging the sterile filters eventually used during clarification increases. High turbidity of the cell culture, such as higher than 2500 NTU, or higher than 3000 NTU may represent a difficult clarification challenge. The cell culture of the present invention has a turbidity comprised between about 500 NTU and about 8000 NTU, more specifically comprised between about 1000 NTU and about 6000 NTU, in certain embodiments the turbidity is comprised between about 1000 NTU and 3000 NTU, more particularly between about 1200 NTU and about 2500 NTU; in other embodiments the turbidity is comprised between about 3000 NTU and 6000 NTU, more particularly between about 3500 NTU and about 5800 NTU. Non limiting examples of the cell culture turbidity include: about 500 NTU, about 800 NTU, about 1000 NTU, about 1200 NTU, about 1400 NTU, about 1600 NTU, about 1800 NTU, about 2000 NTU, about 2200 NTU, about 2400 NTU, about 2600 NTU, about 2800 NTU, about 3000 NTU about 3200 NTU, about 3400 NTU, about 3600 NTU, about 3800 NTU, about 4000 NTU about 4200 NTU, about 4400 NTU, about 4600 NTU, about 4800 NTU, about 5000 NTU, about 5200 NTU, about 5400 NTU, about 5600 NTU, about 5800 NTU, about 6000 NTU. In certain embodiments, the turbidity of the cell culture is less than about 3000 NTU, e.g. it is comprised between 1200 NTU and 2500 NTU, more in particular it is selected from the group comprising about 800 NTU, about 1000 NTU, about 1200 NTU, about 1400 NTU, about 1600 NTU, about 1800 NTU, about 2000 NTU, about 2200 NTU, about 2400 NTU, about 2600 NTU, about 2800 NTU, about 2900 NTU. In certain other embodiments the turbidity of the cell is equal to or greater than about 3000 NTU e.g. equal to or greater than about 3500 NTU, equal to or greater than about 4000 NTU, equal to or greater than about 4500 NTU, equal to or greater than about 5000 NTU, equal to or greater than about 5500 NTU, or comprised between about 3000 NTU and about 8000 NTU, more specifically between about 3000 NTU and about 6000 NTU, even more specifically between about 3500 NTU and about 5800 NTU, more in particular selected from the group comprising about 3000 NTU about 3200 NTU, about 3400 NTU, about 3600 NTU, about 3800 NTU, about 4000 NTU about 4200 NTU, about 4400 NTU, about 4600 NTU, about 4800 NTU, about 5000 NTU about 5200 NTU, about 5400 NTU, about 5600 NTU, about 5800 NTU, about 6000 NTU. The present invention also includes turbidity values at any intermediate value of the above said value.

For a clarification method to be efficient, it is also important that the throughput is maximized. As used herein the terms “throughput” or “loading capacity” or “capacity” are interchangeable and indicate the volume clarified by a clarification operational unit, for instance the volume filtered through a filter, more particularly, the volume normalized by filter's area (L/m²).

The cell culture clarification can start with a primary clarification step. The terms “primary clarification” and “primary recovery” as used herein are interchangeable and refer to the removal of large particles such as whole cells and cell debris. The primary clarification can be followed by a secondary clarification step. The terms “secondary clarification” and “secondary recovery” as used in the present patent application are interchangeable and indicate the removal of smaller particles.

Primary and secondary clarification may require one or more clarification operational unit such as filters, centrifuges, acoustic separator etc., and their combinations.

In one embodiment, the present invention discloses a method for clarifying a cell culture including a biomolecule of interest and having a turbidity between about 1000 NTU and about 6000 NTU characterized by comprising (a) a primary clarification step which removes cell culture material of size equal to or greater than about 0.2 μm and (b) a secondary clarification step comprising filtration which removes cell culture material of size equal to or less than 0.4 μm, wherein said secondary clarification step has a maximum throughput equal to or greater than about 80 L/m2 and wherein said primary and secondary clarification steps lead to a turbidity reduction equal to or greater than about 90%.

In a more particular embodiment of the present invention, the primary clarification step is the group comprising depth filtration, centrifugation and flocculation.

In an even more particular embodiment of the present invention, the primary clarification step is a depth filtration.

As used herein, the term “depth filtration” refers to a technology that exploits filters with a certain porosity to retain particles of a medium throughout the medium, rather than just on the surface. Examples of depth filters used for instance in biopharmaceutical industry include single-use devices in the form of a lenticular disks or cartridges which contain the filter sheets at small scale. Disks or cartridges can be assembled into multilayer housings at larger scales. Depth filters are typically composed of cellulose fibers and filter aids like diatomaceous earth or perlite bound together into a polymeric resin. In some cases, cellulose fibers can be replaced by fully synthetic polymeric fibers like polyacrylic or polystyrene. These fibers form a three-dimensional network with a certain porosity. The permeability and retention characteristics of the filters are directly correlated with the length and compaction of those fibers. The porosity of the filters matrix can vary along with the filter depth, allowing the coverage a wide range of particle exclusion. Non limiting examples of depth filters useful for the present invention comprise filters made of cellulose and/or resin, and/or synthetic media and/or polypropylene and/or filters aids. A depth filter can be suitable for both primary and/or secondary recovery, or for primary or secondary recovery only. Filters suitable for primary and/or secondary recovery are also known as “single filters”. Single filters can be applied alone to carried out both the primary and the secondary recovery, or they can be used as filters for primary recovery and coupled with the subsequent use of a filter for secondary recovery; or they can be used as filters for secondary recovery for instance after a centrifugation step for primary recovery. In the present invention single filters are also referred as “SF”, filters for primary clarification are referred as “PCF” and filters for secondary clarification are referred as “SCF”.

In the present invention, the terms “nominal exclusion range”, “exclusion range”, “retention range” and “grade” are interchangeable and they refer to the ability of a filter to retain particles of a specific size. For instance a filter with exclusion range comprised between 1 μm and 10 μm it is able to retain particles of size comprised between 1 μm and 10 μm; particles bigger than the highest limit of the exclusion range do not pass into the filter but remain in the surface. The exclusion range of a depth filter can vary from above 60 μm to less than 0.1 μm. The porosity, namely the pore size, of the filters allow a size exclusion mechanism in order to remove large particles like whole cells and cell debris. To a certain extent, depth filters can also remove colloidal contaminants, like HCP or DNA, which are smaller than the pore size. The removal of these contaminants is allowed by the positively charged filter matrix. The positive charge of the filter matrix is brought by the polymeric resins that bind together the filter components. The electrokinetic interactions between the filter matrix and the colloidal particles leads to the removal of those small contaminants whereas bigger particles are trapped by the porosity of the filter matrix.

In one aspect of the present invention, the primary clarification is performed by a first depth filter selected from the group comprising depth filters for primary clarification and single filters. In another aspect of the present invention the first depth filter has an exclusion range equal to or greater than about 0.1 μm and equal to or less than about 50 μm, more particularly the exclusion range is comprised between about 0.25 μm and about 30 μm. In certain particular embodiments the exclusion range is comprised between about 1 μm and about 20 μm, or comprised between about 5 μm and about 30 μm, preferably comprised between about 6 μm and about 30 μm, or comprised between about 0.5 μm and about 10 μm, preferably comprised between about 0.55 μm and about 8 μm, or comprised between about 0.2 μm and about 2 μm, or comprised between about 1.5 μm and about 10 μm, or comprised between about 0.7 μm and about 5 μm, or comprised between about 0.25 μm and about 5 μm. In a specific aspect the first depth filter has an exclusion range selected from the group comprising at least about 0.1 μm, at least about 0.2 μm, at least about 0.5 μm, at least about 0.7 μm, at least about 1 μm, at least about 1.5 μm, at least about 2 μm, at least about 5 μm, at least about 7 μm, at least about 10 μm, at least about 20 μm, at least about 30 μm. The present invention also includes first depth filters with an exclusion range at any intermediate value of the above said ranges.

In another particular embodiment of the present invention, the primary clarification step is centrifugation.

As used herein the term “centrifugation” refers to a technology that allows the separation of particles by subjecting the cell culture to centrifugal force, and wherein the particles are separated according to their density.

According to one aspect of this invention centrifugation may be run at a relative centrifugal field comprised between about 100 G and e about 15000 G, more specifically at a relative centrifugal field selected from the group comprising about 100 G, about 200 G, about 400 G, about 500 G, about 600 G, about 800 G, about 1000 G, about 1200 G, about 1400 G, about 1500 G, about 1600 G, about 1800 G, about 2000 G, about 2200 G, about 2400 G, about 2500 G, about 2600 G, about 2800 G, about 3000 G, about 3200 G, about 3400 G, about 3500 G, about 3600 G, about 3800 G, about 4000 G, about 4200 G, about 4400 G, about 5500 G, about 5600 G, about 5800 G, about 6000 G, about 6200 G, about 6400 G, about 6500 G, about 6600 G, about 6800 G, about 7000 G, about 7200 G, about 7400 G, about 7500 G, about 7600 G, about 7800 G, about 8000 G, about 8200 G, about 8400 G, about 8500 G, about 8600 G, about 8800 G, about 9000 G, about 9200 G, about 9400 G, about 9500 G, about 9600 G, about 9800 G, about 10000 G, about 9200 G, about 9400 G, about 9500 G, about 9600 G, about 9800 G, about 10000 G, about 12000 G, about 12500 G, about 13000 G, about 13100 G, about 13500 G, about 14000 G, about 14500 G, about 15000 G. Centrifugation according to the present invention may be run in a continuous flow or for a set time. In certain aspects of the present invention centrifugation is run for a time comprised between 10 sec and 20 minutes, in particular for a time selected from the group comprising: about 10 sec, about 30 sec, about 1 min, about 5 min, about 10 min, about 12 min, about 15 min, about 17 min, about 20 min. In a more specific aspect of this invention, centrifugation is run at a relative centrifugal field comprised between about 100 G and e about 6000 G, more in particular between about 500 G and 3000 G for a time comprised between 10 sec and 20 minutes. In a more particular aspect the centrifugation is run at a relative centrifugal field selected from the group comprising at least about 100 G, at least about 500 G, at least about 1000 G, at least about 1500 G, at least about 2000 G, at least about 2500 G, at least about 3000 G, at least about 3500 G, at least about 4000 G, at least about 5000 G for a time selected from the group comprising at least about 10 sec, at least about 30 sec, at least about 1 min, at least about 5 min, at least about 10 min, at least about 15 min, at least about 20 min. In some embodiments, centrifugation is run at a relative centrifugal field equal to or greater than about 500 G and equal to or less than 3000 G for a time comprised between 1 and 10 min. In a preferred embodiment, centrifugation is run at a relative centrifugal field of about 500 G or of about 3000 G, more in particular centrifugation is run at a relative centrifugal field of about 500 G for about 1 min or at a relative centrifugal field of about 3000 G for about 5 min. The present invention also includes centrifugation run at relative centrifugal fields at any intermediate value of the above said range.

In another particular embodiment of the present invention, the primary clarification step is flocculation.

The term “flocculation” refers to the aggregation, precipitation and/or agglomeration of insoluble particles caused by the addition of a suitable flocculating agent to a suspension. By increasing the particle size of the insoluble components present in the suspension, the efficiency of solid/liquid separations, such as by filtration, is improved. Flocculation of a cell culture leads to the formation of “floccules” which comprise host cell impurities such as cell material including cells, cell debris, host cell proteins, DNA and other components present therein. The flocculation process can be triggered by different methods including the reduction of the cell culture pH or the addition of flocculants (also known as flocculating agents). Non limiting examples of flocculants include: calcium phosphate, caprylic acid, divalent cations or positively charged polymers like polyamine, chitosan or polydiallyldimethylammonium chloride (e.g. pDADMAC), which induce the particles aggregation due to their interaction with the negatively charged surface of cells and cell debris.

In one embodiment of the present invention, the flocculating agent is a positively charged polymer. In a more particular embodiment the flocculating agent is polydiallyldimethylammonium chloride, and it is added to the cell culture at a percentage equal to or greater than about 0.005 v/v and equal to or less than 0.1% v/v. In particular polydiallyldimethylammonium chloride it is added to the cell culture at a percentage selected from the group comprising about 0.005% v/v, about 0.01% v/v, about 0.03% v/v, about 0.05% v/v, about 0.08% v/v, about 0.1% v/v. In a preferred embodiment, polydiallyldimethylammonium chloride it is added to the cell culture at a percentage of about 0.03% v/v. The present invention also includes the addition of a flocculation agent to a cell culture at any intermediate percentage value of the above said range.

In another particular embodiment of the present invention, the secondary clarification step is carried out by a second depth filtration.

In one aspect of the present invention, the second clarification is performed by a second depth filter selected from the group comprising depth filters for secondary clarification, single filters and post-flocculation filters. In another aspect of the present invention the second depth filter has an exclusion range comprised between about 0.01 μm and about 10 μm, more in particular comprised between about 0.05 μm and about 5 μm, even more in particular comprised between about 0.1 μm and about 4 μm, in a further particular embodiment the second depth filter exclusion range in comprised between about 0.2 μm and about 3.5 μm. In a specific aspect, the second depth filter has an exclusion range equal to or less than about 3.5 μm, or equal to or less than about 3 μm, or equal to or less than about 2 μm, or equal to or less than about 1 μm, or equal to or less than about 0.5 μm, or equal to or less than about 0.2 μm, or equal to or less than about 0.1 μm. The present invention also includes second depth filters with an exclusion range at any intermediate value of the above said values.

In one aspect of the present invention, the secondary clarification step has a the maximum throughput is equal to or greater than about 50 L/m², or equal to or greater than about 60 L/m², or equal to or greater than about 80 L/m², or equal to or greater than about 100 L/m², or equal to or greater than about 150 L/m², or equal to or greater than about 200 L/m², or equal to or greater than about 250 L/m², or equal to or greater than about 300 L/m², or equal to or greater than about 350 L/m², or equal to or greater than about 400 L/m², or equal to or greater than about 450 L/m², or equal to or greater than about 500 L/m², or equal to or greater than about 550 L/m², or equal to or greater than about 600 L/m²; more specifically the maximum throughput is selected from the group comprising about 50 L/m2, about 55 L/m², about 60 L/m², about 65 L/m², about 67 L/m², about 70 L/m², about 75 L/m², about 80 L/m², about 85 L/m², about 90 L/m², about 95 L/m², about 100 L/m², about 125 L/m², about 150 L/m², about 175 L/m², about 200 L/m², about 225 L/m², about 250 L/m², about 275 L/m², about 300 L/m², about 325 L/m², about 350 L/m² about 375 L/m², about 400 L/m², about 425 L/m², about 450 L/m², about 475 L/m², about 500 L/m², about 525 L/m², about 550 L/m², about 575 L/m², about 600 L/m², about 625 L/m², about 650 L/m², about 675 L/m², about 700 L/m², about 725 L/m², about 750 L/m², about 775 L/m², about 800 L/m², about 825 L/m², about 850 L/m², about 875 L/m², about 900 L/m², about 925 L/m², about 950 L/m², about 975 L/m², about 1000 L/m². The present invention also comprises maximum throughput at any intermediate values of the ones said above.

In another aspects, the primary and the secondary clarification lead to a turbidity reduction equal to or greater than about 80%, or equal to or greater than about 85%, preferably equal to or greater than about 90%, more preferably equal to or greater than about 95%, even more preferably equal to or greater than about 98%, most preferably equal to or greater than about 99%; more specifically the turbidity reduction is selected from the group comprising about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%. The present invention also comprises turbidity reduction at any intermediate values of the ones said above.

In certain embodiments of the present invention, when the cell culture has a turbidity less than about 3000 NTU, e.g. equal to or less than about 2500 NTU, equal to or less than about 2000 NTU, or comprised between 800 NTU and 3000 NTU, more specifically between 1200 NTU and 2500 NTU, following the primary and secondary clarification the maximum throughput is equal to or greater than about 100 L/m², or equal to or greater than about 150 L/m², or equal to or greater than about 200 L/m², or equal to or greater than about 250 L/m², or equal to or greater than about 300 L/m², or equal to or greater than about 350 L/m², or equal to or greater than about 400 L/m², or equal to or greater than about 450 L/m², or equal to or greater than about 500 L/m², or equal to or greater than about 550 L/m², or equal to or greater than about 600 L/m², and the turbidity reduction is equal to or greater than about 90%, specifically equal to or greater than about 95%, even more specifically equal to or greater than about 98%, preferably equal to or greater than about 99%. More specifically the maximum throughput is selected from the group comprising: about 100 L/m², about 125 L/m², about 150 L/m², about 175 L/m², about 200 L/m², about 225 L/m², about 250 L/m², about 275 L/m², about 300 L/m², about 325 L/m², about 350 L/m², about 375 L/m², about 400 L/m², about 425 L/m², about 450 L/m², about 475 L/m², about 500 L/m², about 525 L/m², about 550 L/m², about 575 L/m², about 600 L/m², and the turbidity reduction is selected from the group comprising about 90%, about 91%, about 92%, about 93%, about 94%, about 95%, about 96%, about 97%, about 98%, about 99%, about 100%. The present invention also comprises turbidity, throughput and turbidity reduction at any intermediate values of the ones said above.

In other embodiments, when the cell culture has a turbidity equal to or greater than about 3000 NTU, e.g. equal to or greater than about 3500 NTU, equal to or greater than about 4000 NTU, equal to or greater than about 4500 NTU, equal to or greater than about 5000 NTU, equal to or greater than about 5500 NTU, or comprised between about 3000 NTU and about 8000 NTU, more specifically between about 3000 NTU and about 6000 NTU, even more specifically between about 3500 NTU and about 5800 NTU, and when a combination of a depth filter for primary clarification and a depth filter for secondary clarification is used, the maximum throughput is equal to or greater than about 50 L/m², or equal to or greater than about 60 L/m², or equal to or greater than about 80 L/m², or equal to or greater than about 100 L/m², or equal to or greater than about 150 L/m², or equal to or greater than about 200 L/m², or equal to or greater than about 250 L/m², or equal to or greater than about 300 L/m², or equal to or greater than about 350 L/m², or equal to or greater than about 400 L/m², or equal to or greater than about 450 L/m², or equal to or greater than about 500 L/m², or equal to or greater than about 550 L/m², or equal to or greater than about 600 L/m², and the turbidity reduction is equal to or greater than about 90%, specifically equal to or greater than about 95%, even more specifically equal to or greater than about 98%, preferably equal to or greater than about 99%. In a particular embodiment when the cell culture has a turbidity equal to or greater than about 3000 NTU, and clarification is performed with a first depth filter with exclusion range comprised between about 5 μm and about 30 μm, or comprised between about 0.5 μm and about 10 μm, followed by a second depth filter with an exclusion range equal to or less than about 3.5 μm, the maximum throughput is equal to or greater than about 80 L/m² and the turbidity reduction is equal to or greater than about 99%. More specifically the maximum throughput is selected from the group comprising: about 50 L/m2, about 55 L/m², about 60 L/m², about 65 L/m², about 67 L/m², about 70 L/m², about 75 L/m², about 80 L/m², about 85 L/m², about 90 L/m², about 95 L/m², about 100 L/m², about 110 L/m², about 120 L/m², about 130 L/m², about 140 L/m², about 150 L/m², about 160 L/m², about 170 L/m², about 180 L/m², about 190 L/m², about 200 L/m², about 225 L/m², about 250 L/m², about 275 L/m², about 300 L/m², about 325 L/m², about 350 L/m², about 375 L/m², about 400 L/m², about 425 L/m², about 450 L/m², about 475 L/m², about 500 L/m², about 525 L/m², about 550 L/m², about 575 L/m², about 600 L/m², and the turbidity reduction is selected from the group comprising about 90%, about 91%, about 92%, about 93%, about 94%, about 95%, about 96%, about 97%, about 98%, about 99%, about 99.5% about 99.8%, about 100%. The present invention also comprises turbidity, throughput and turbidity reduction at any intermediate values of the ones said above.

In certain embodiments of the present invention, when a combination of a depth filter for primary clarification and a depth filter for secondary clarification is used, the surface ratio between the first and the second depth filter is selected from the group comprising 1:1, 2:1, 1:2.

In another embodiment of the present invention, when the turbidity of the cell culture is less than 3000 NTU, the primary clarification step and the secondary clarification step are performed by a single filter with an exclusion range comprised between about 1 μm and about 20 μm, so as the maximum throughput is equal to or greater than about 80 L/m², or equal to or greater than about 100 L/m², or equal to or greater than about 110 L/m², or equal to or greater than about 150 L/m² and the turbidity reduction is equal to or greater than about 90%, specifically equal to or greater than about 95%, even more specifically equal to or greater than about 98%. More specifically the maximum throughput is selected from the group comprising: about 80 L/m², about 85 L/m², about 90 L/m², about 95 L/m², about 100 L/m², about 110 L/m², about 115 L/m², about 120 L/m², about 130 L/m², about 140 L/m2, about 150 Um′, and the turbidity reduction is selected from the group comprising about 90%, about 91%, about 92%, about 93%, about 94%, about 95%, about 96%, about 97%, about 98%, about 98.5%, about 99%, about 99.5% about 99.8%, about 100%. The present invention also comprises turbidity, throughput and turbidity reduction at any intermediate values of the ones said above.

In one embodiment of the present invention, the primary clarification is preceded by a cell culture pretreatment step of acoustic wave separation. In another aspect of the present invention, acoustic wave separation may be used for the primary clarification step.

The term “acoustic wave separation” (AWS) as used herein refers to a technology that allows reducing the turbidity of a cell culture by forcing said cell culture into at least a flow channel where an acoustic force is applied that traps the cells and induce their clumping, which is followed by cells dropping by the gravitational force. In one embodiment of the present invention the AWS is performed by Cadence™ Acoustic Wave Separator system by Pall.

In one embodiment of the present invention the second clarification is followed by a bioburden reduction.

The term “bioburden reduction” as used herein refers to the reduction of the number of microorganisms in the fluid obtained after the primary and secondary clarification. Normally bioburden reduction is considered the final step of the clarification process and comprises one or more steps of sterile purification. In one embodiment of this invention the bioburden reduction is performed by at least one sterile filter having exclusion range equal to or less than about 0.5 μm. In a more specific embodiment, the bioburden reduction is performed by at least one sterile filter having exclusion range from about 0.2 μm to about 0.45 μm.

In one aspect of the present invention, the secondary depth filtration may be followed by a further filtration performed by a membrane absorber with exclusion range equal to less than 0.2 μm.

At the end of the clarification process, the clarified cell culture may be further subjected to one or more steps of purification to isolate and recover the biomolecule of interest. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful.

The current invention also discloses a cell culture subjected to the method of anyone of the preceding claims.

The current invention also discloses a process of production of a drug substance comprising the steps of:

(i) seeding cells expressing a biomolecule of interest in a cell culture medium;
(ii) culturing said cells for a period comprised between 10 and 18 days, for instance for a period selected from the group comprising about 10 day, about 11 days, about 12 days, about 13 days, about 14 days, about 15 days, about 16 days, about 17 days, about 18 days, preferably for 14 days;
(iii) subjecting the obtained cell culture to the clarification method according to the present invention;
(iv) add excipients to the purified biomolecule of interest.

Unless otherwise defined, all terms used in this disclosure, including technical and scientific terms, have the meaning as commonly understood by a person skilled in the art to which this disclosure belongs. By means of further guidance, term definitions are included to better appreciate the teaching of the present disclosure.

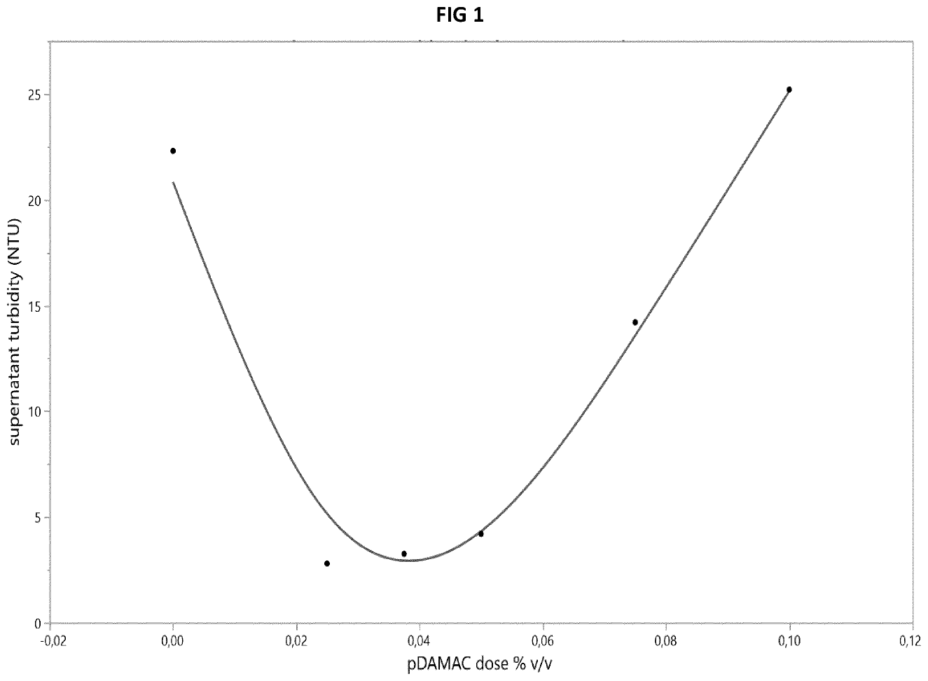

FIG. 1: pDADMAC dosing study allowing to determine the optimal impact on turbidity

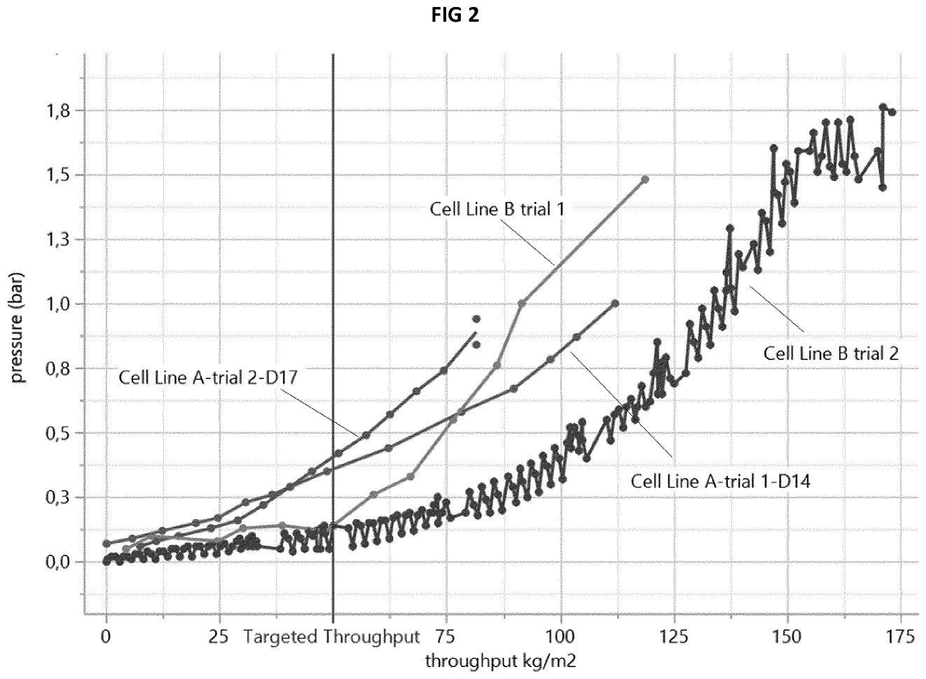

FIG. 2: Correlation of pressure (bar) and throughput (L/m²) in the clarification process

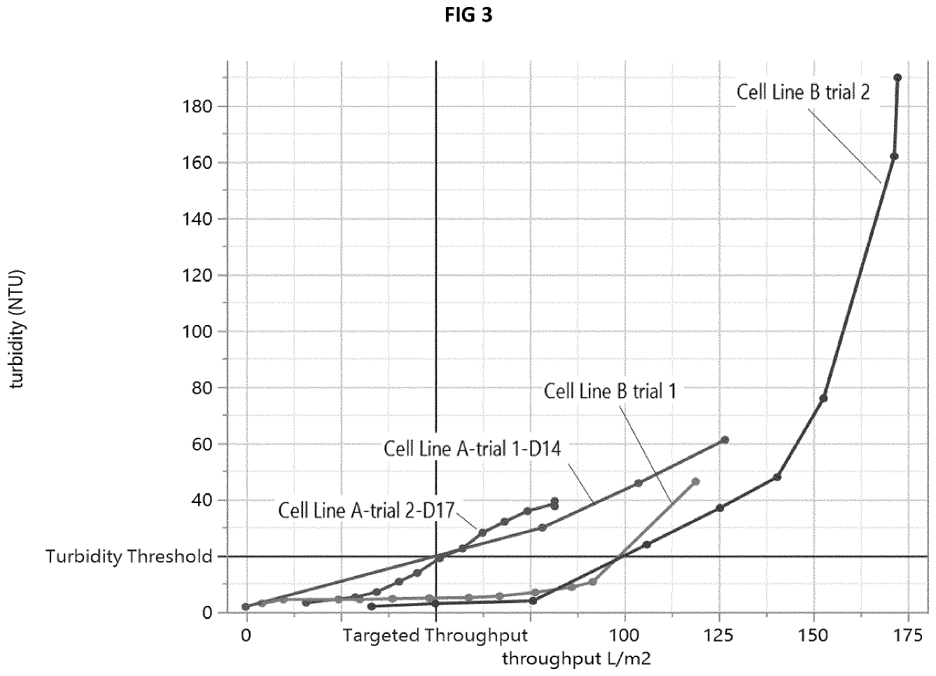

FIG. 3: Correlation of turbidity (NTU) and throughput (L/m²) in the clarification process

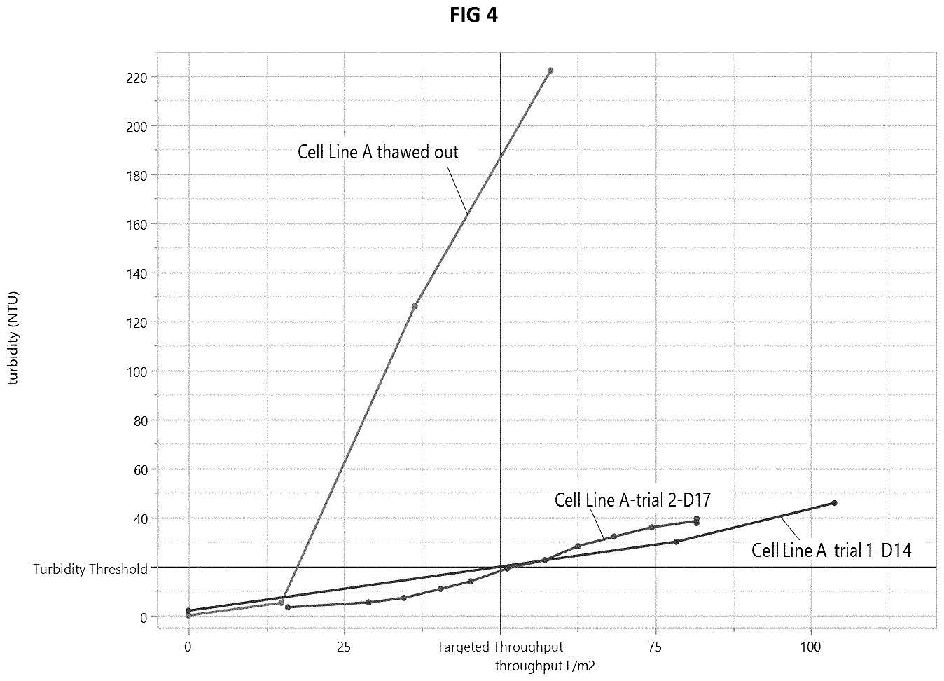

FIG. 4: Correlation of turbidity (NTU) and throughput (L/m²): limitation with worst case CCF

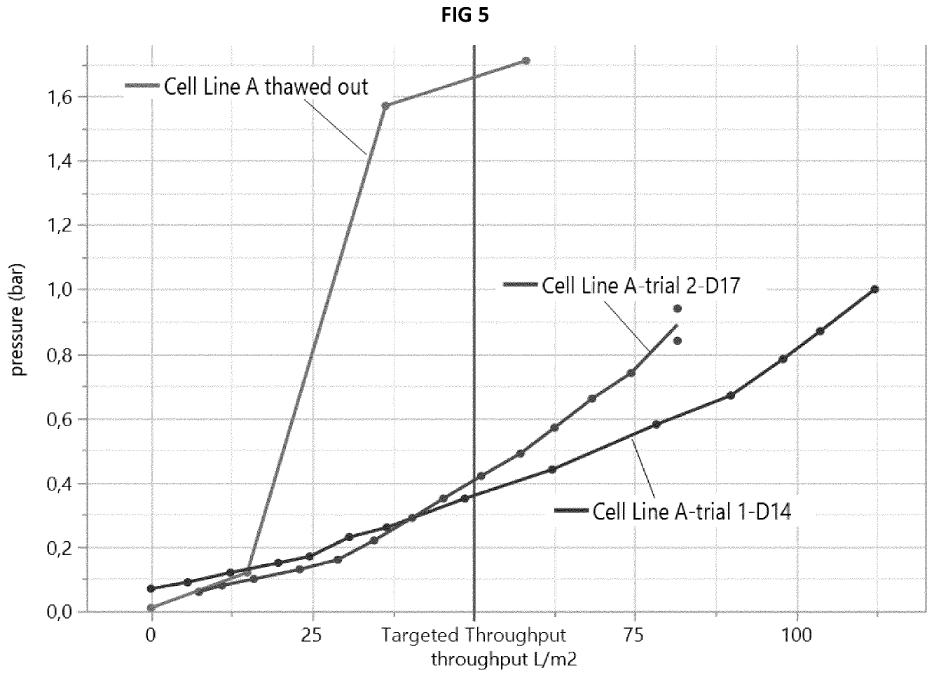

FIG. 5: Correlation of pressure (bar) and throughput (L/m²): limitation with worst case CCF

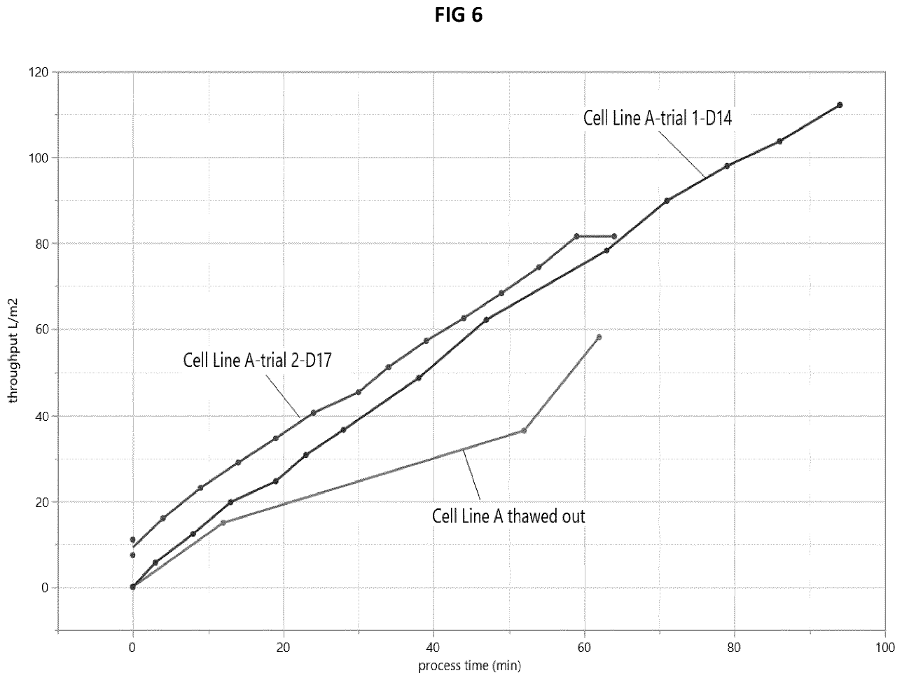

FIG. 6: Correlation of throughput (L/m²) and time (min) in order to determine limitations with worst case CCF

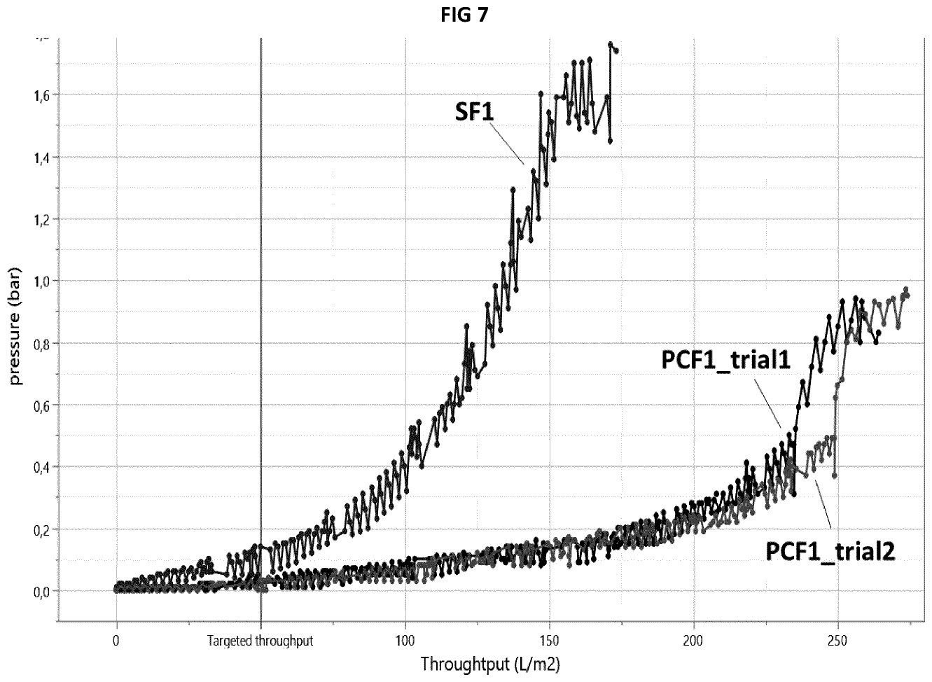

FIG. 7: Correlation of pressure (bar) and throughput (L/m²) in the characterization of primary filters

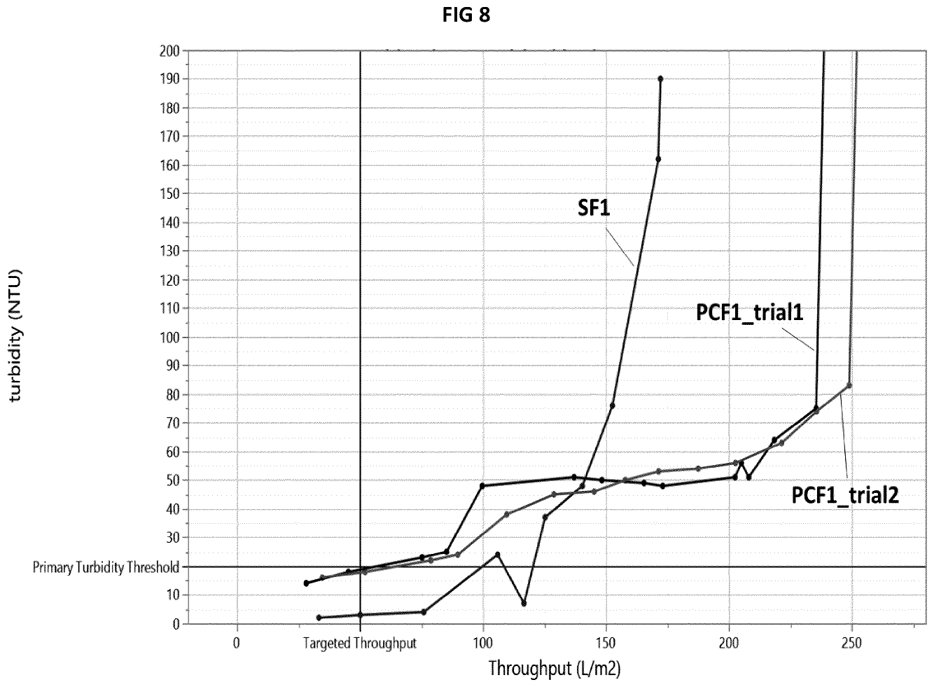

FIG. 8: Correlation of turbidity (NTU) and throughput (L/m²) in the characterization of primary filters

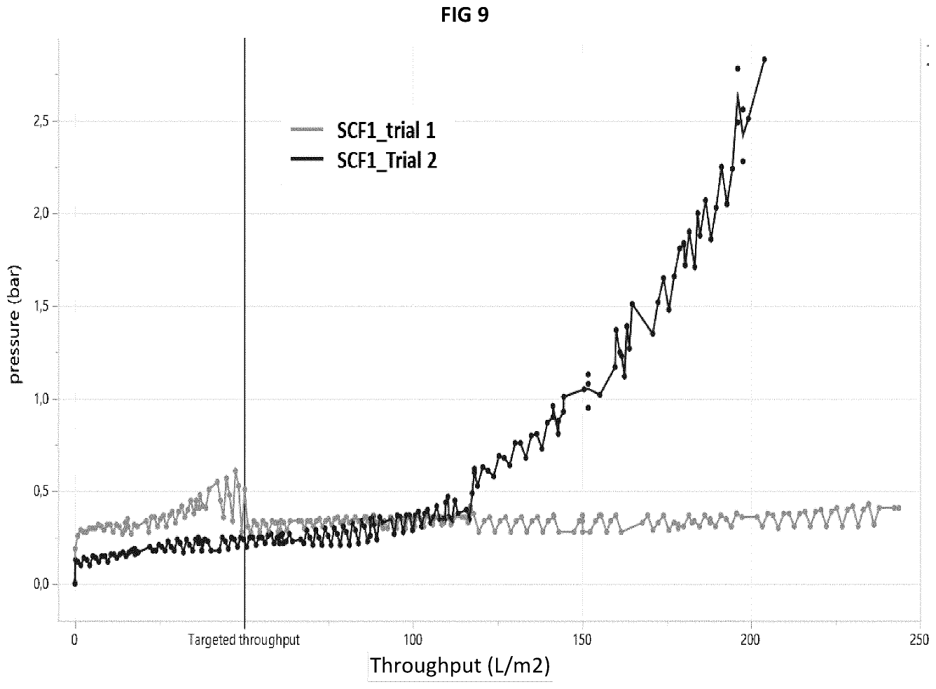

FIG. 9: Correlation of pressure (bar) and throughput (L/m²) in secondary filters characterization

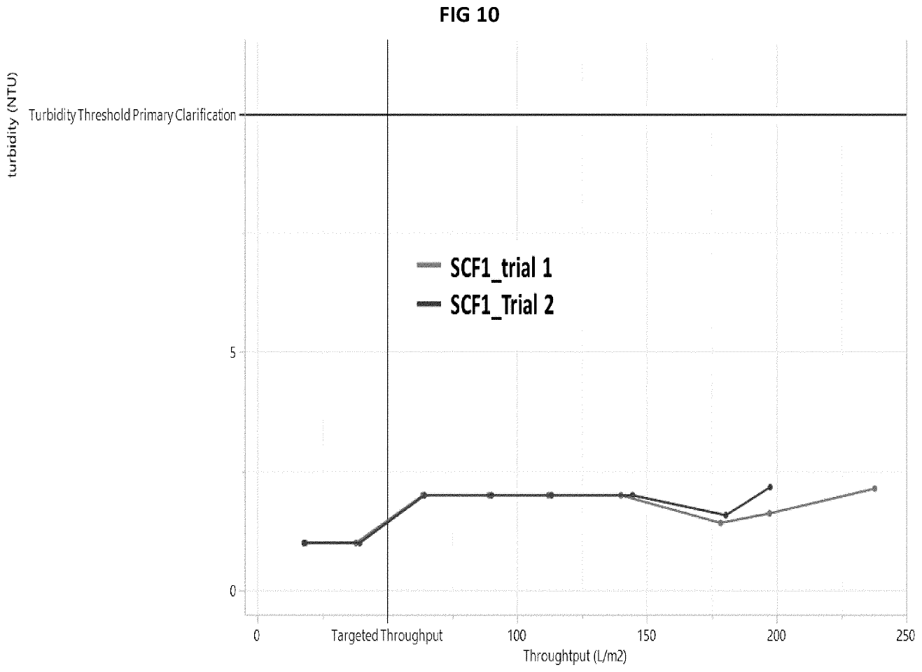

FIG. 10: Correlation of turbidity (NTU) and throughput (L/m²) in secondary filters characterization

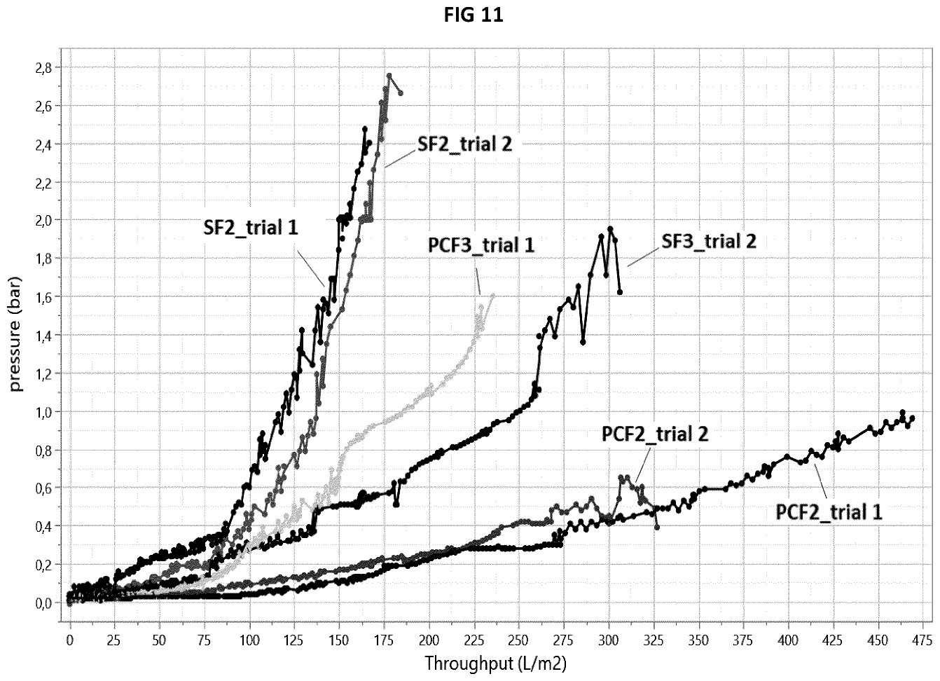

FIG. 11: Correlation of pressure (bar) and throughput (L/m²) in primary clarification filters characterization with representative case CCF

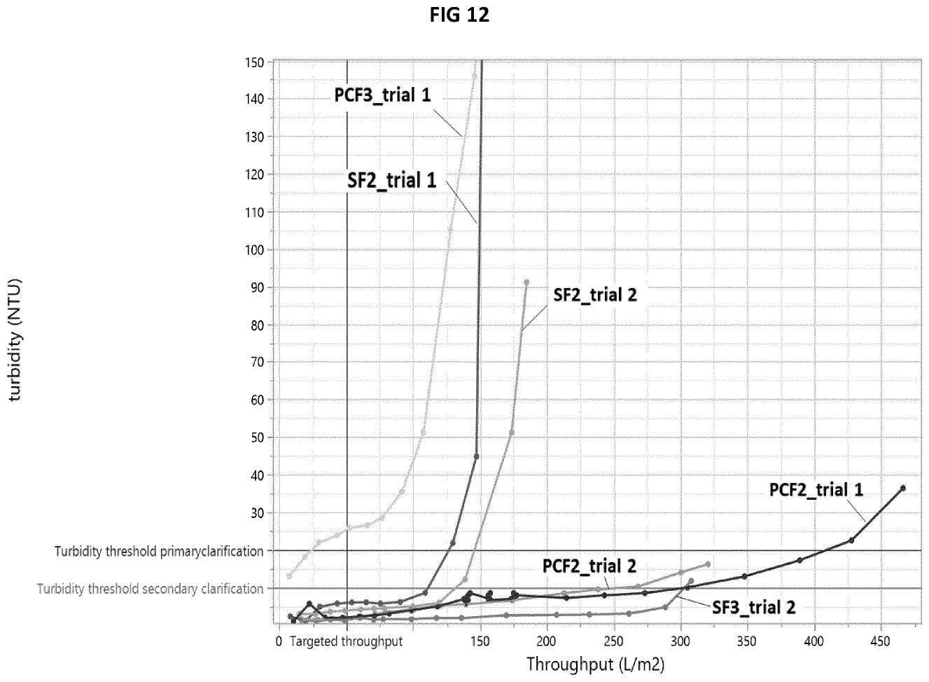

FIG. 12: Correlation of turbidity (NTU) and throughput (L/m²) in primary clarification filters characterization with representative case CCF

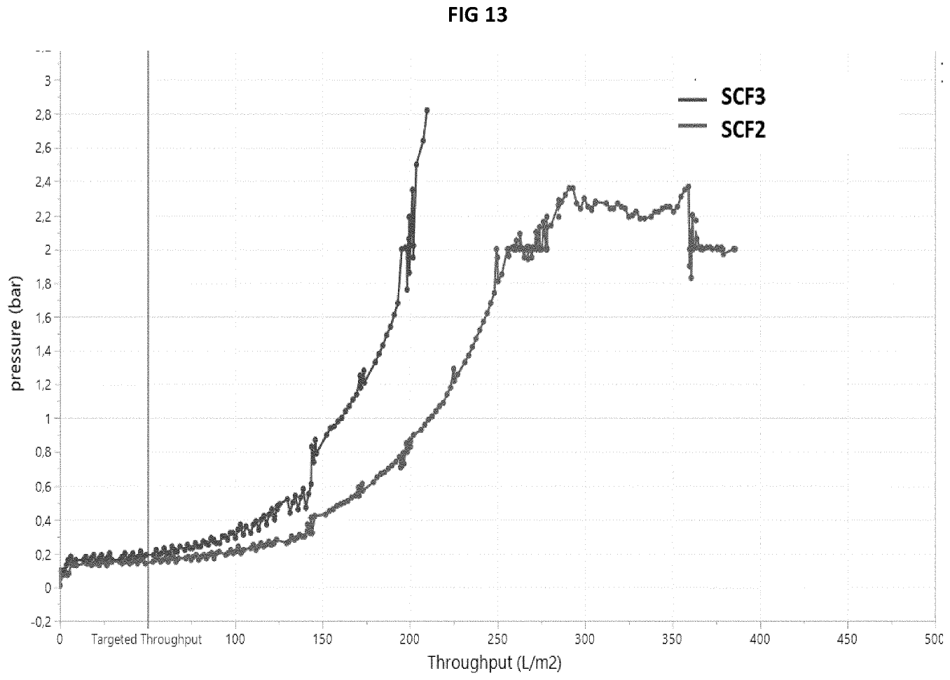

FIG. 13: Correlation of pressure (bar) and throughput (L/m²) in secondary clarification filters characterization with representative case CCF

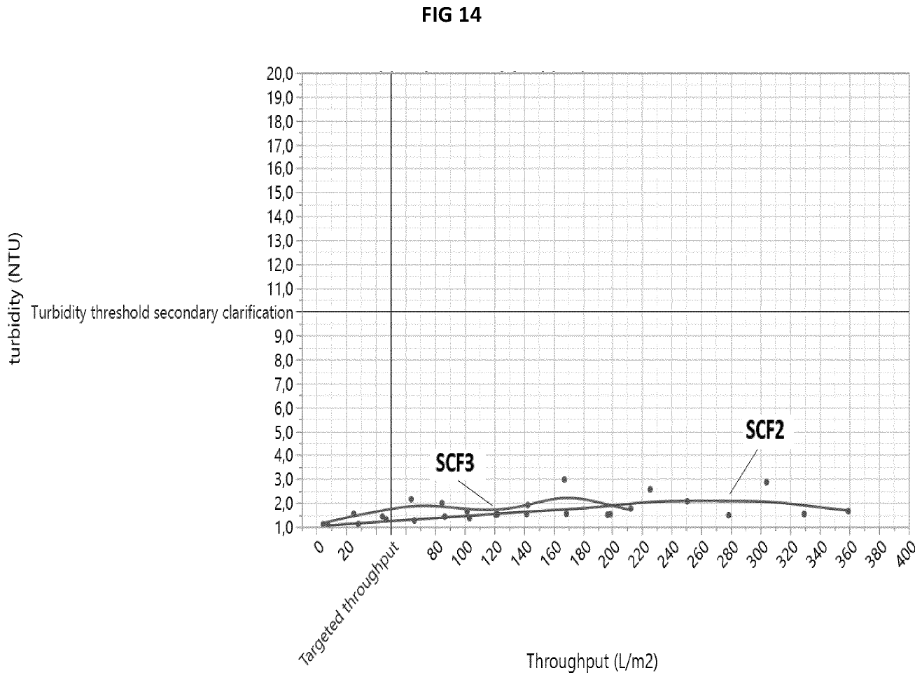

FIG. 14: Correlation of turbidity (NTU) and throughput (L/m²) in secondary clarification filters characterization with representative case CCF

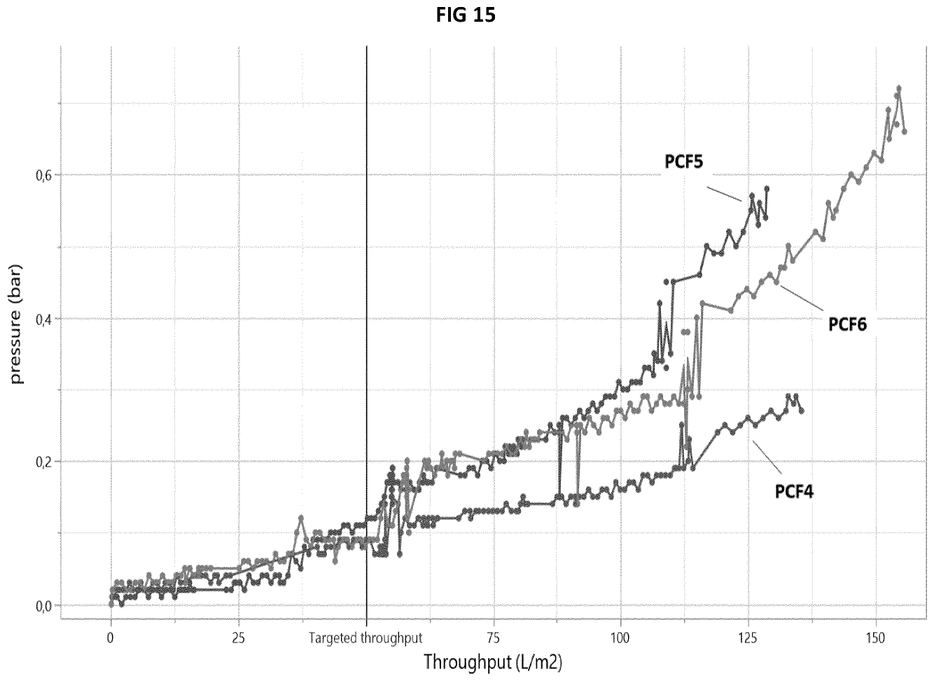

FIG. 15: Correlation of pressure (bar) and throughput (L/m²) in primary clarification filters characterization

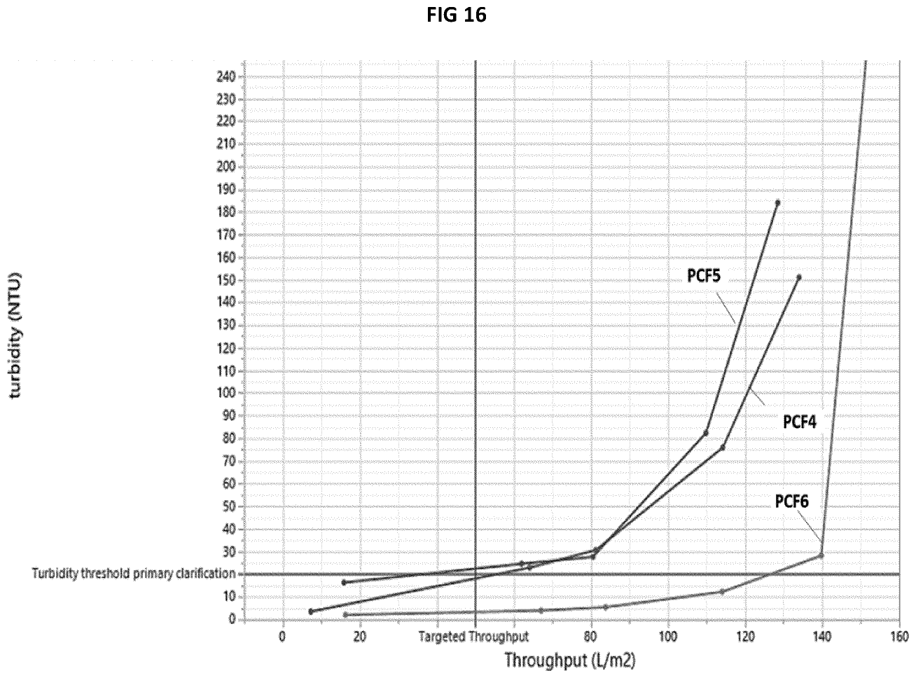

FIG. 16: Correlation of turbidity (NTU) and throughput (L/m²) in primary clarification filters characterization

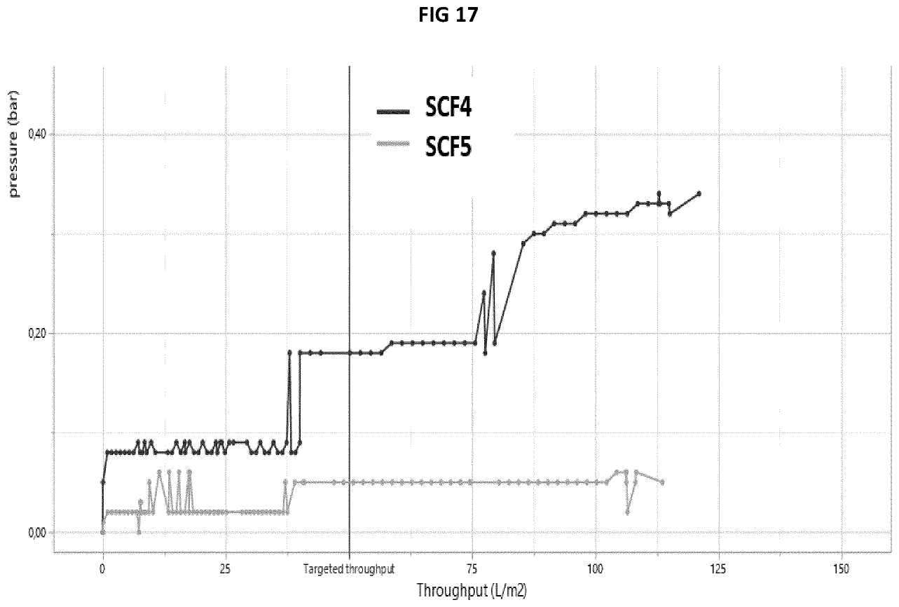

FIG. 17: Correlation of pressure (bar) and throughput (L/m²) in secondary clarification filters characterization

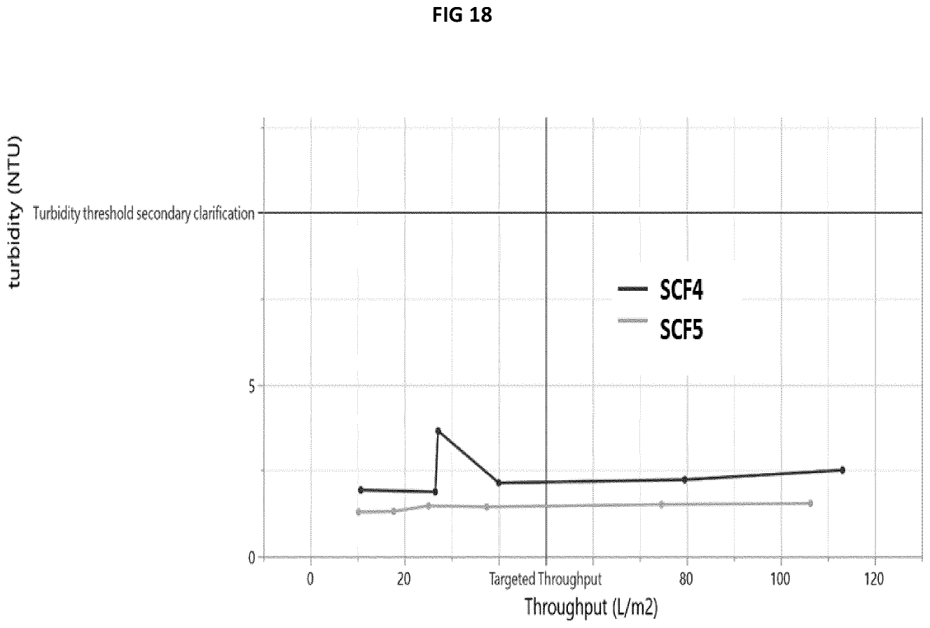

FIG. 18: Correlation of turbidity (NTU) and throughput (L/m²) in secondary clarification filters characterization

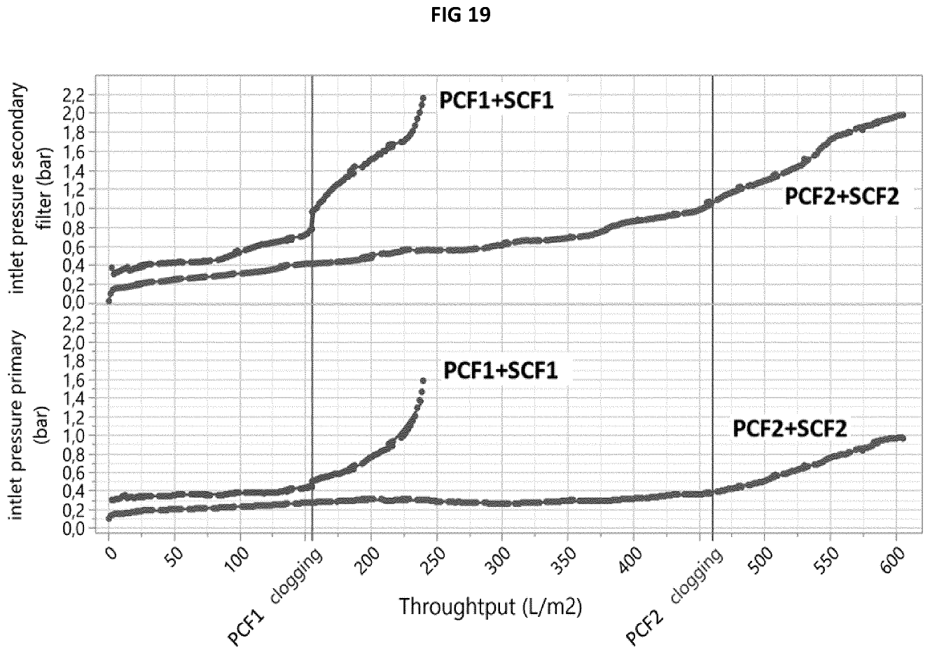

FIG. 19: Primary and secondary inlet pressure (bar) vs throughput (L/m²) for filter combinations PCF2+SCF2 and PCF1+SCF1

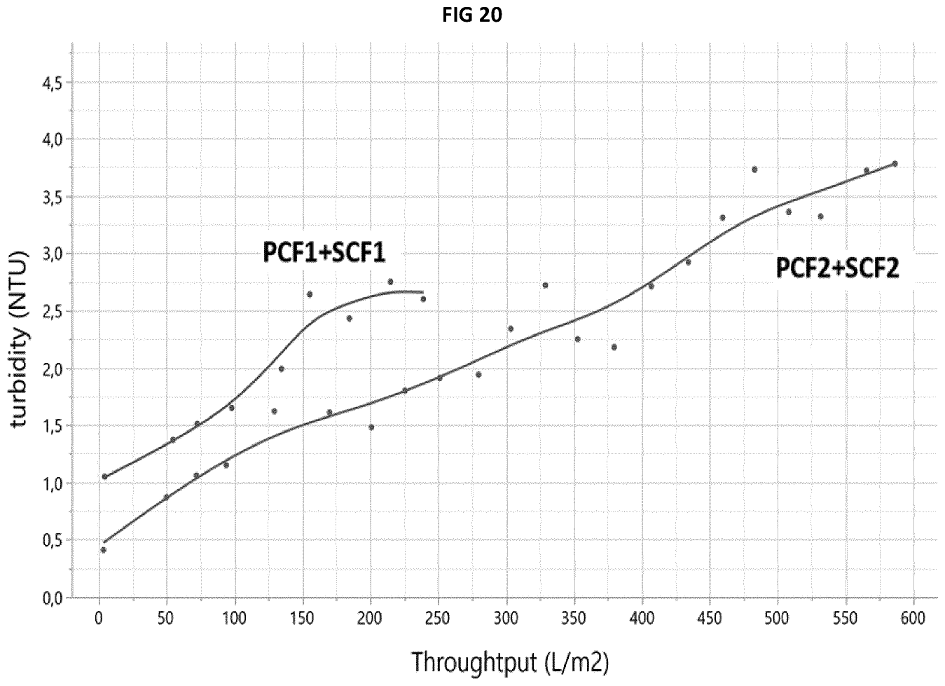

FIG. 20: Turbidity (NTU) vs throughput (L/m²) for filters combinations PCF2+SCF2 and PCF1+SCF1

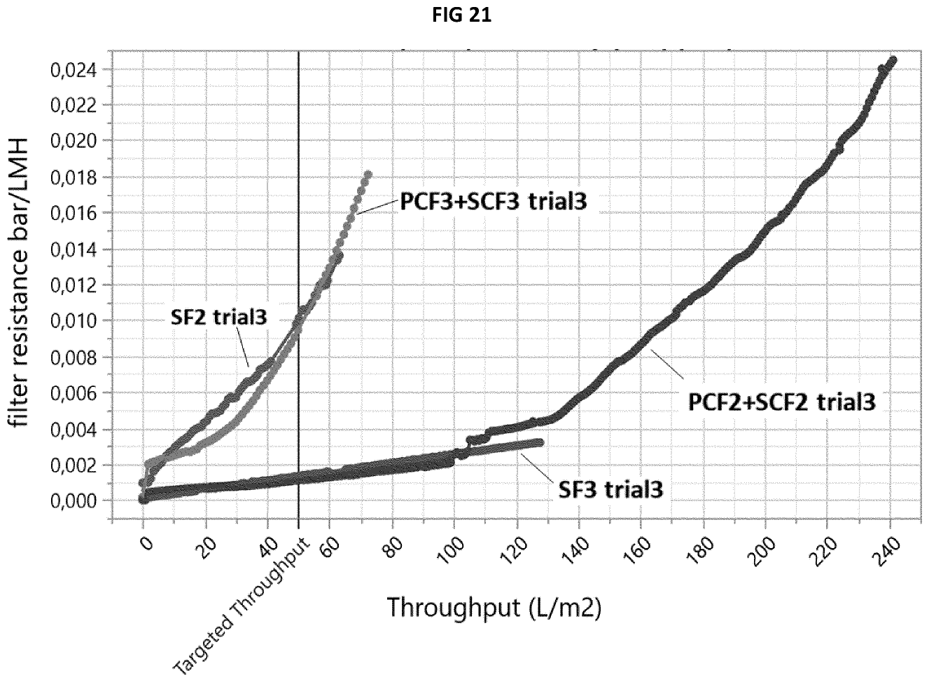

FIG. 21: Correlation of filter resistance (bar/LMH) and throughput (L/m²) in primary and secondary clarification filters characterization with challenging CCF

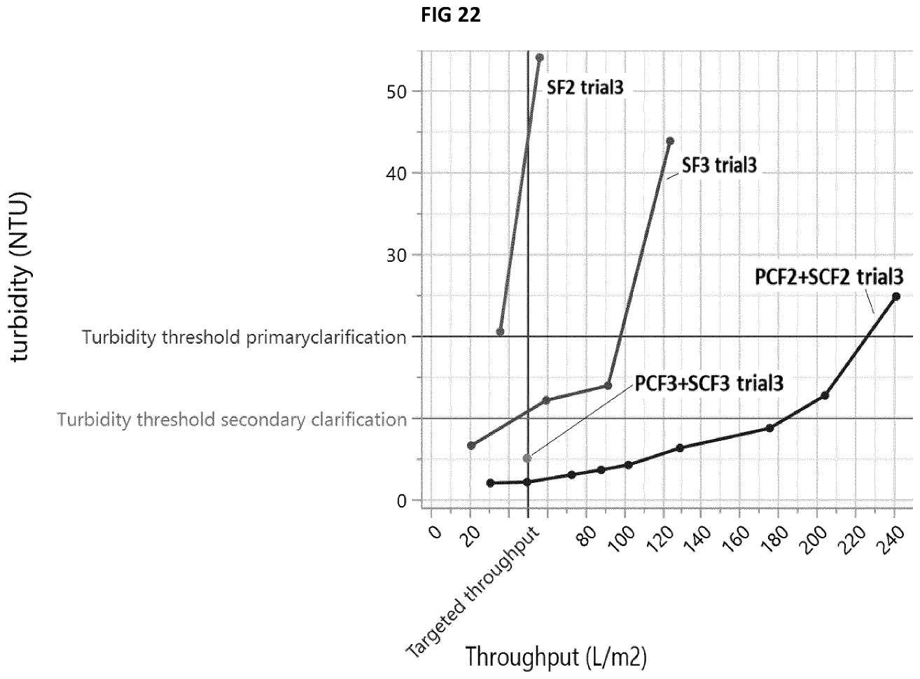

FIG. 22: Correlation of turbidity (NTU) vs throughput (L/m2) in primary and secondary clarification filters characterization with challenging CCF

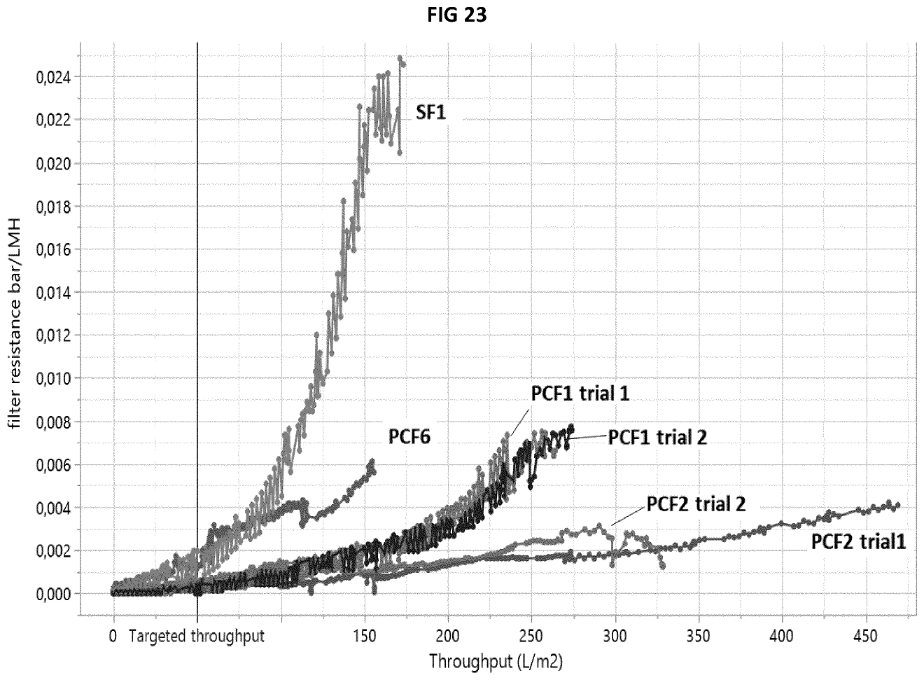

FIG. 23: Correlation of filter resistance (bar/LMH) and throughput (L/m²) in filters global comparison

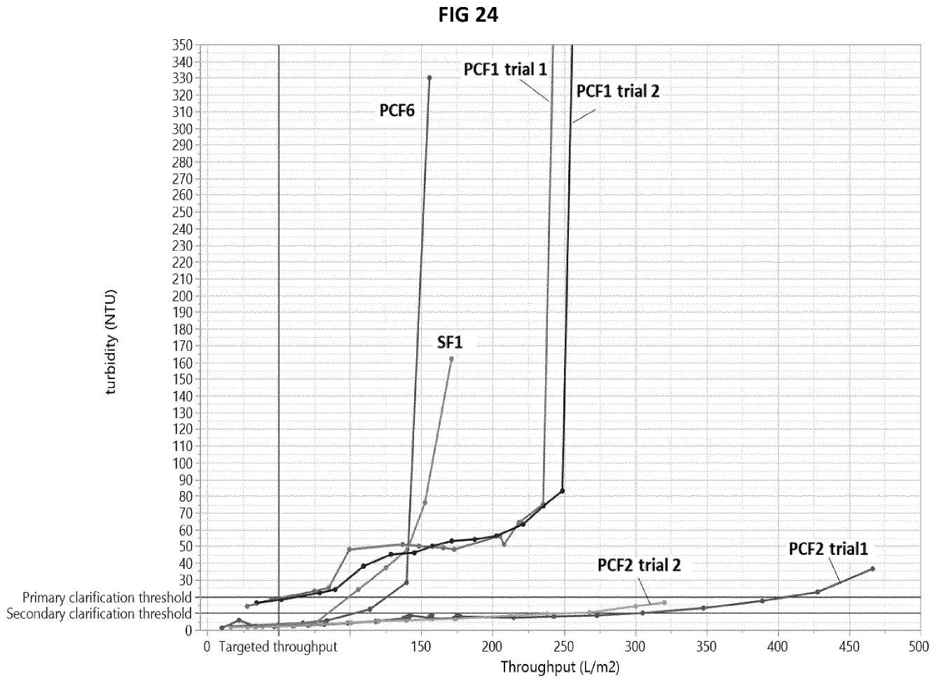

FIG. 24: Correlation of turbidity (NTU) and throughput (L/m²) in filters global comparison

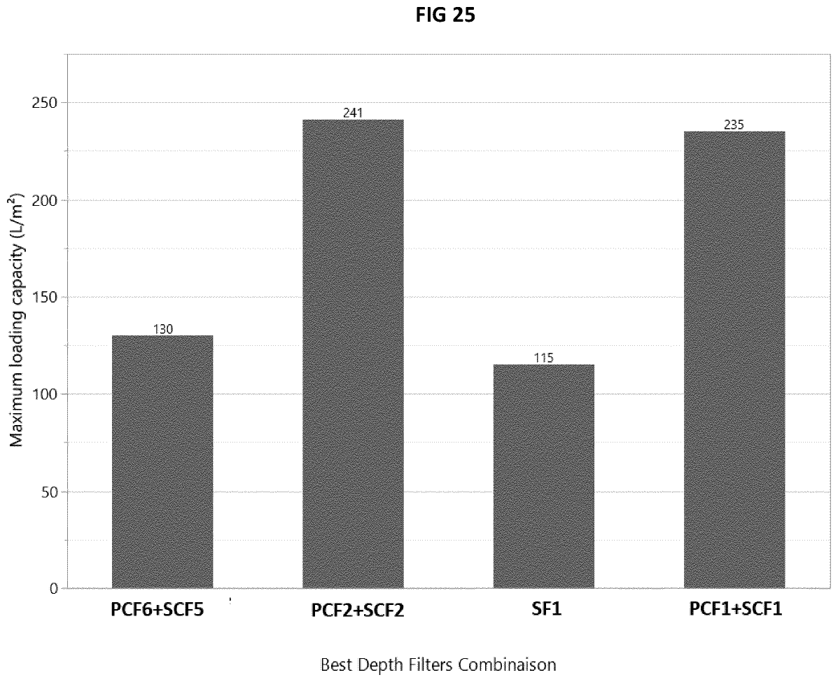

FIG. 25: Correlation of turbidity (NTU) and RCF (G) in the evaluation of the centrifugation step

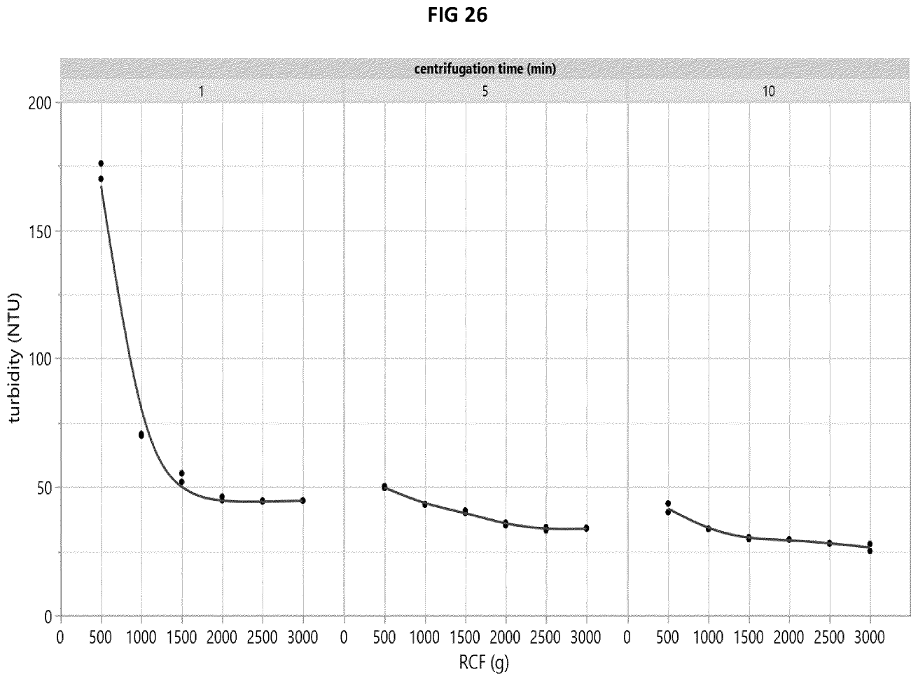

FIG. 26: Comparison of the maximum loading capacity between best selected filters

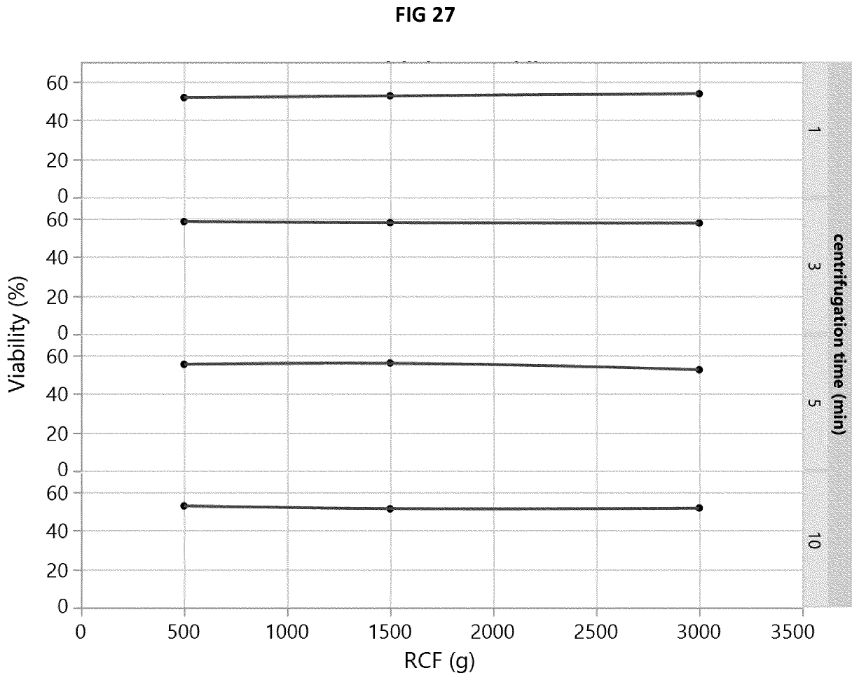

FIG. 27: Correlation of viability (%) and RCF (G) in the evaluation of the centrifugation step

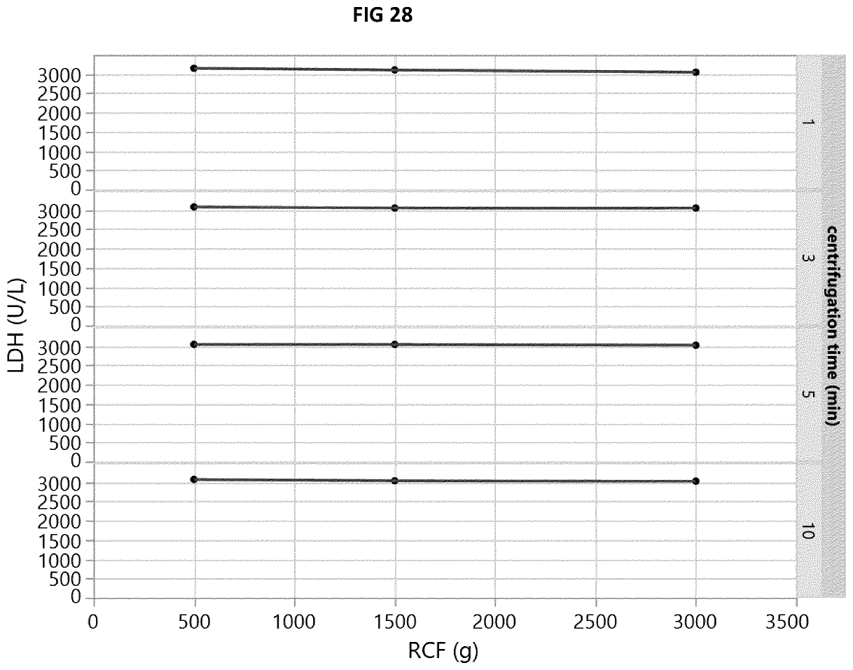

FIG. 28: Correlation of LDH (U/L) and RCF (G) in the evaluation of the centrifugation step

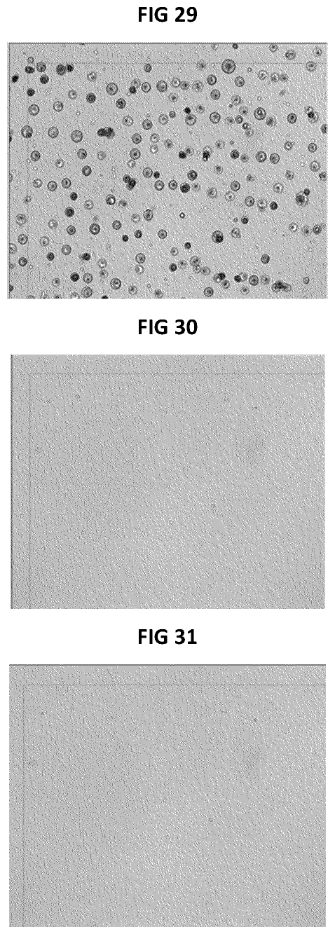

FIG. 29: Image taken from thawed CCF directly after thawing

FIG. 30: Image taken from CCF after centrifugation at 500 g for 1 min

FIG. 31: Image taken from CCF after centrifugation at 3000 g for 5 min

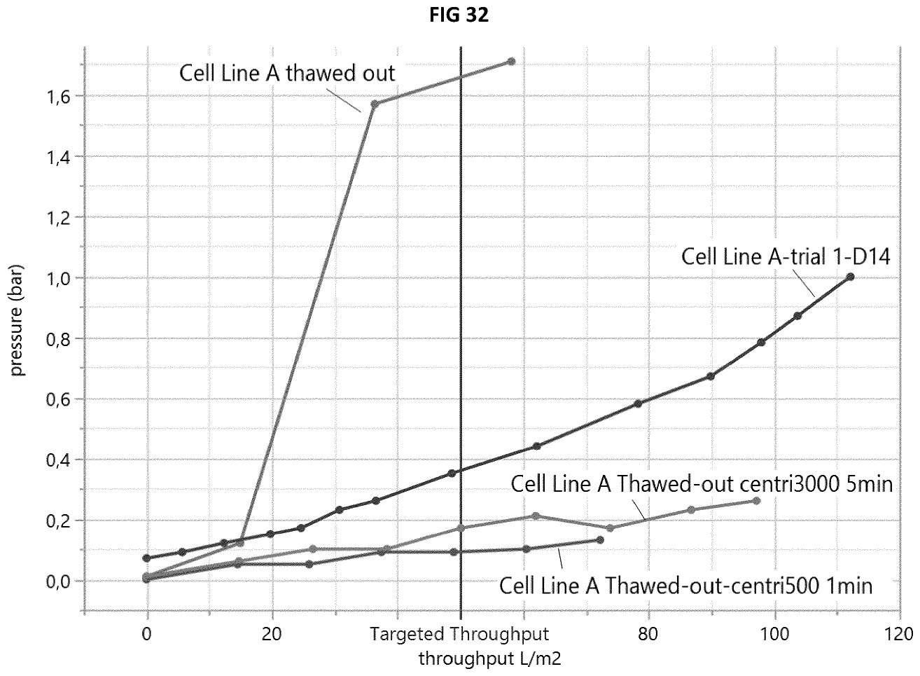

FIG. 32: Correlation of pressure (bar) and throughput (L/m²) using worst case CCF and the SF1 filter as part of the centrifugation step evaluation

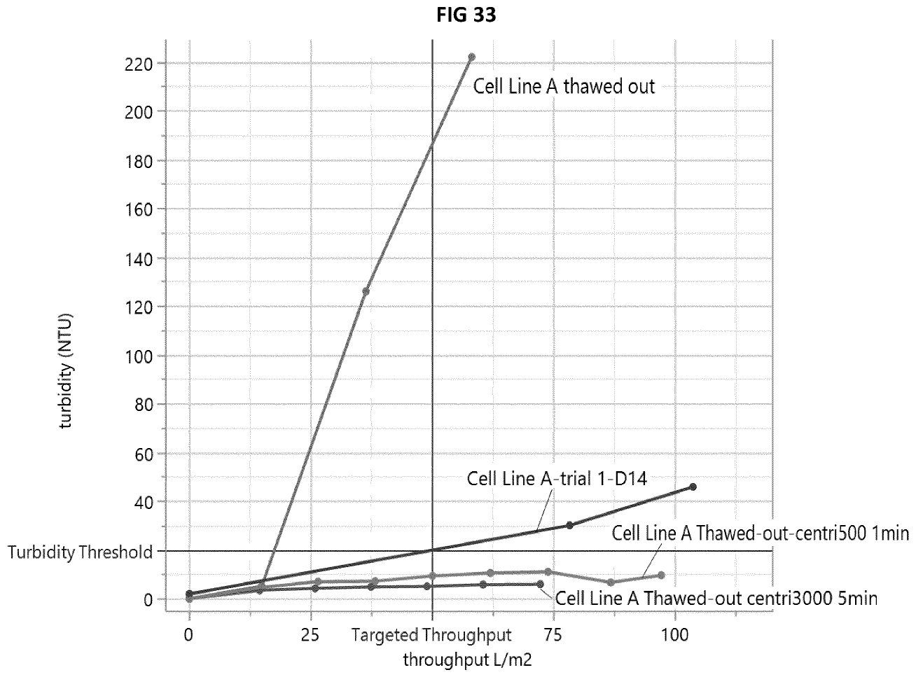

FIG. 33: Correlation of turbidity and throughput (L/m²) using worst case CCF and the SF1 filter as part of the centrifugation step evaluation

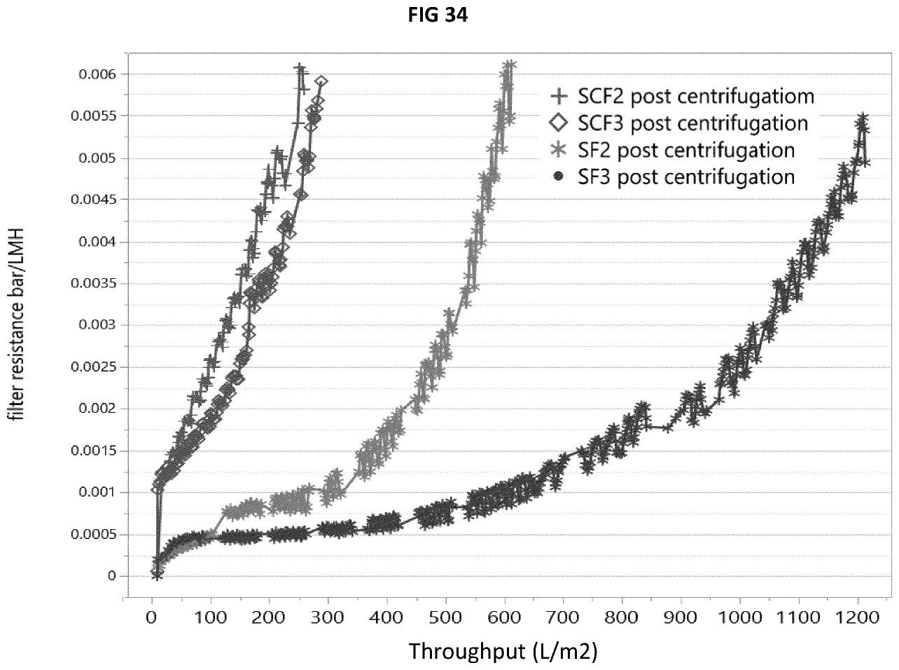

FIG. 34: Correlation of pressure (bar) and throughput (L/m²) in the centrifugation evaluation

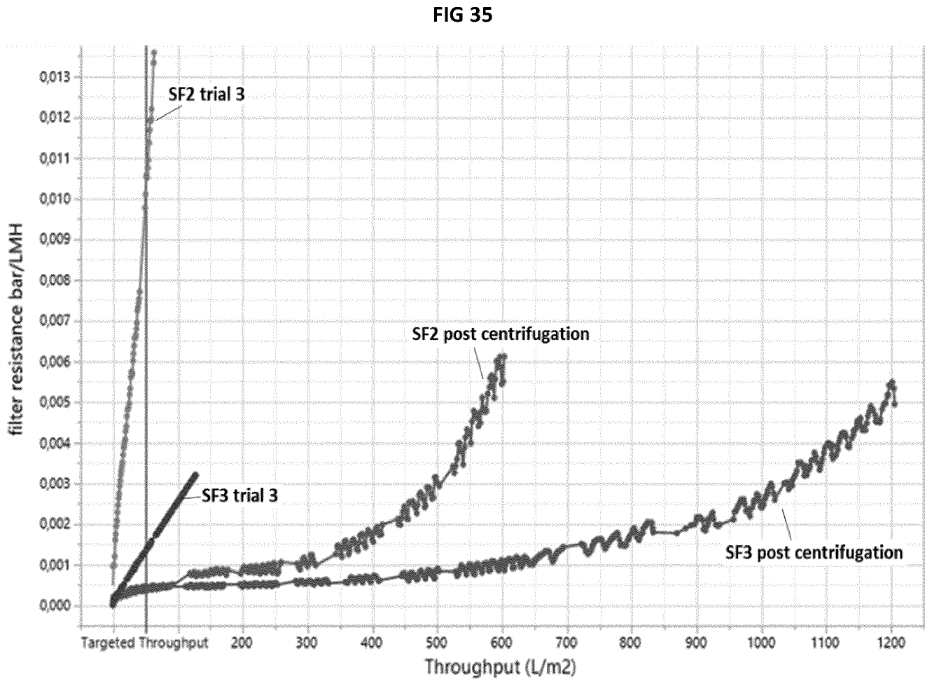

FIG. 35: Correlation of filter resistance (bar/LMH) and throughput (L/m²) in filters with or without prior centrifugation of the CCF

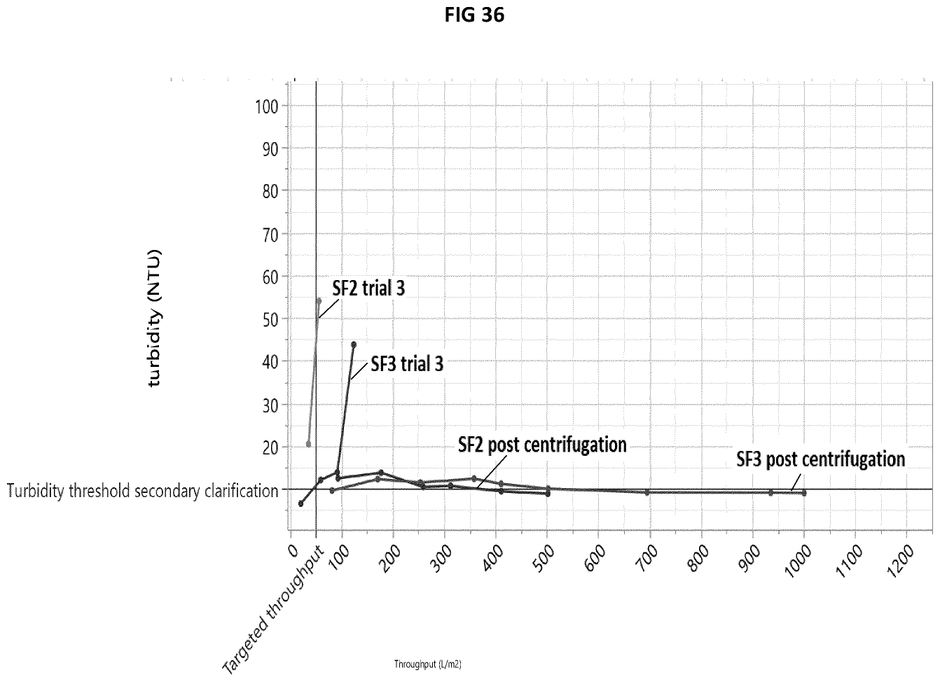

FIG. 36: Correlation of turbidity (NTU) and throughput (L/m²) in filters with or without prior centrifugation of the CCF

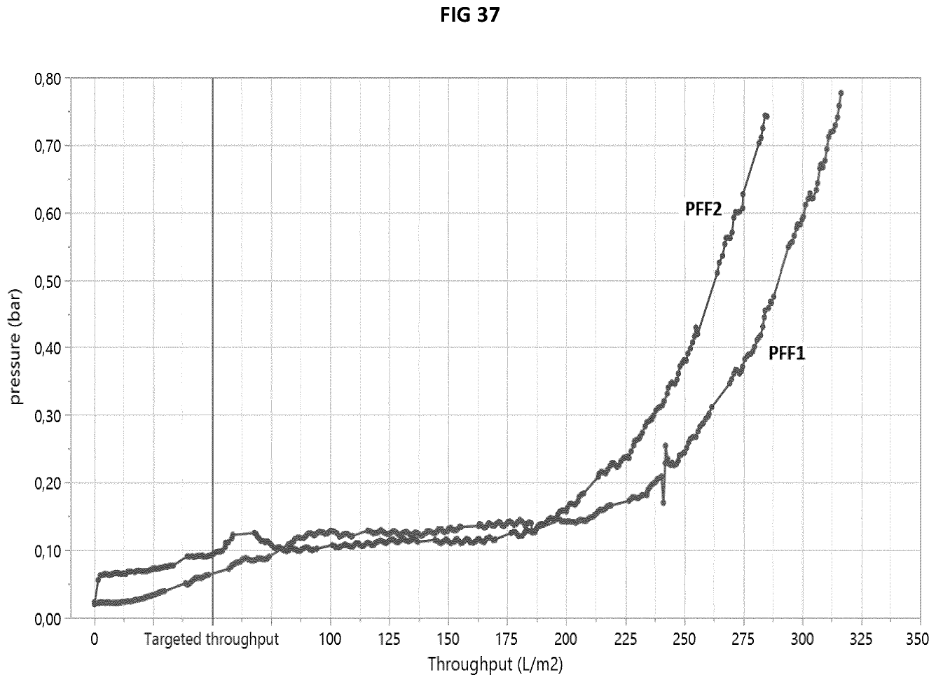

FIG. 37: Correlation of pressure (bar) and throughput (L/m²) in flocculation study

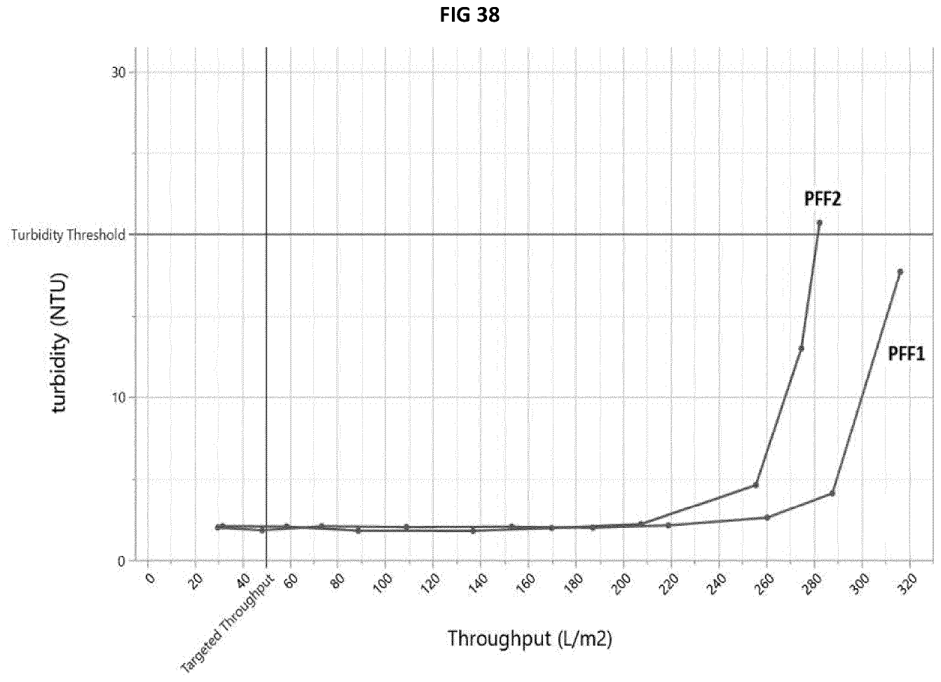

FIG. 38: Correlation of turbidity (NTU) and throughput (L/m²) in flocculation study

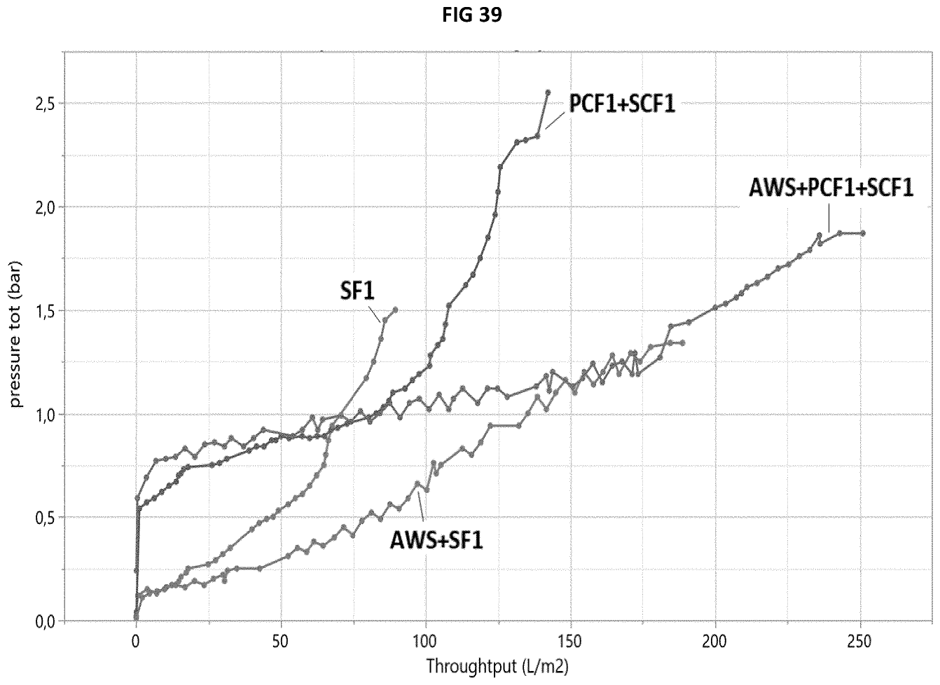

FIG. 39: Impact of AWS on depth filtration, pressure (bar) vs Throughput (L/m²)

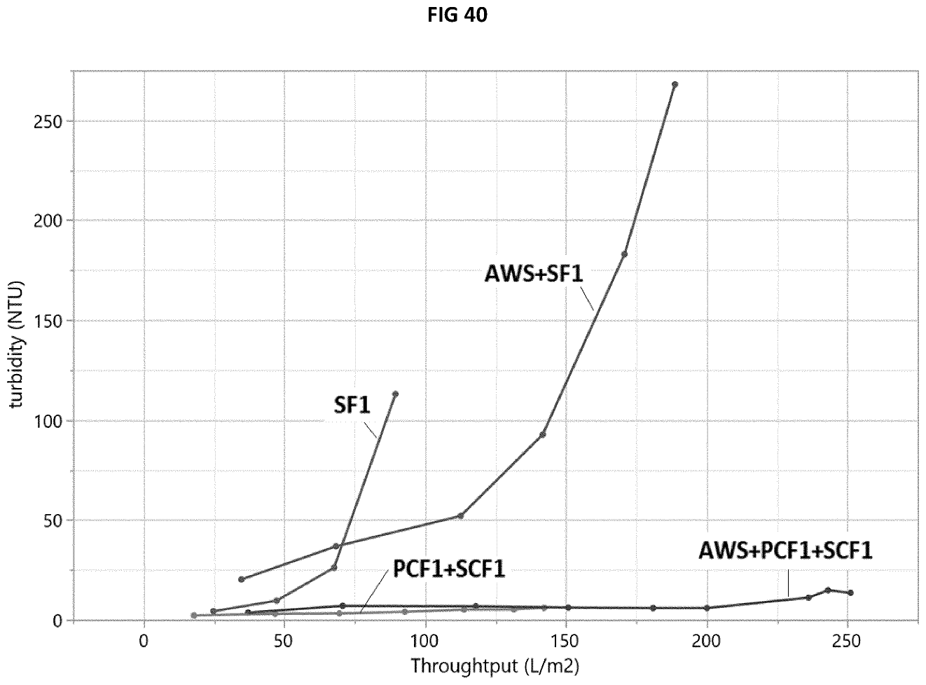

FIG. 40: Impact of AWS on depth filtration, turbidity (NTU) vs Throughput (L/m²)

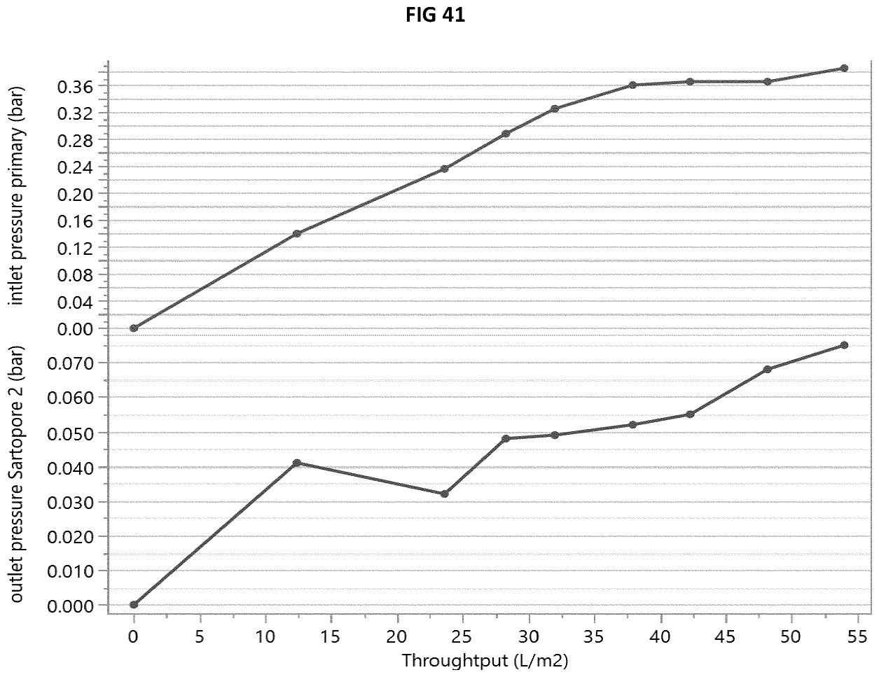

FIG. 41: Pressure monitoring for SF1 filter

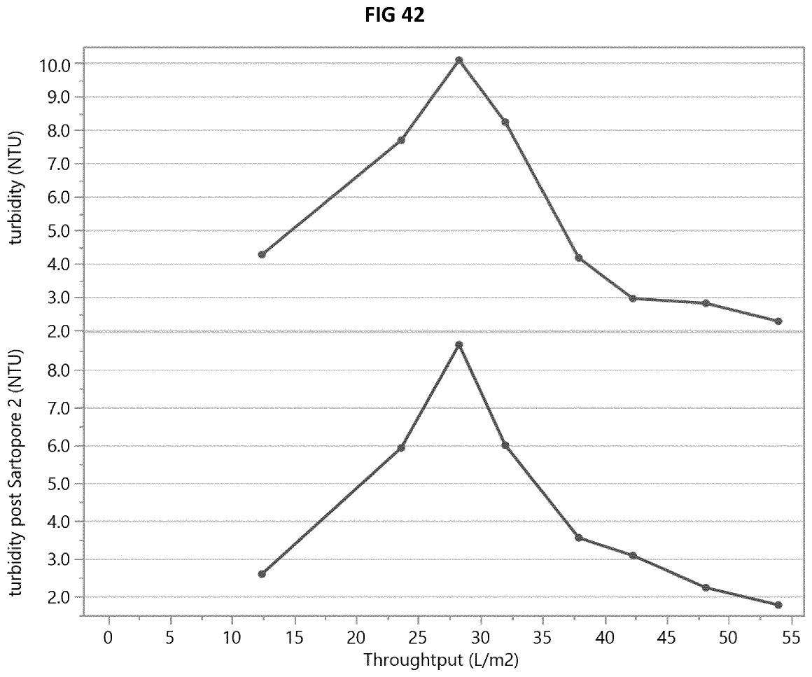

FIG. 42: Turbidity monitoring for SF1 filter

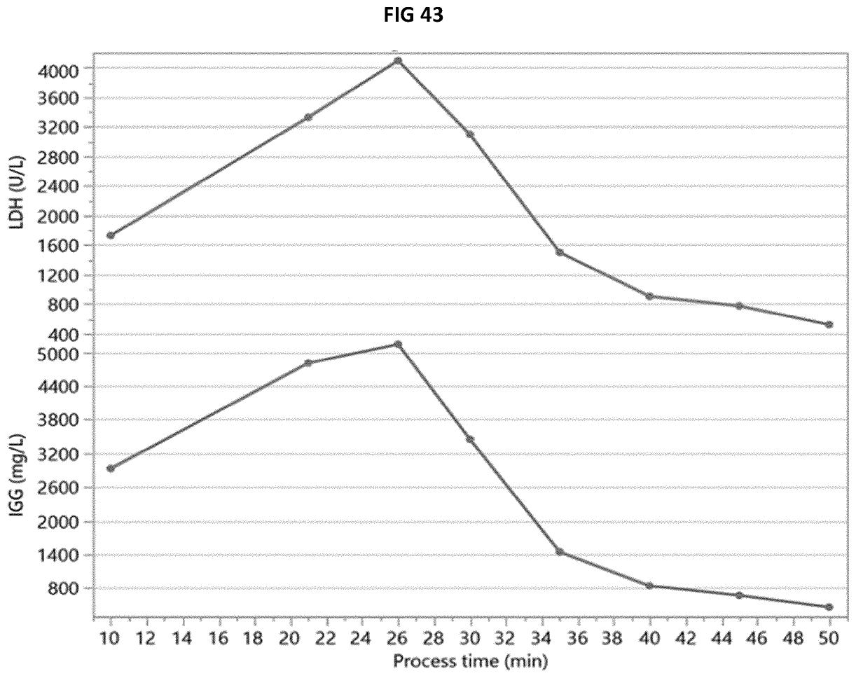

FIG. 43: LDH and titer monitoring for SF1 filter

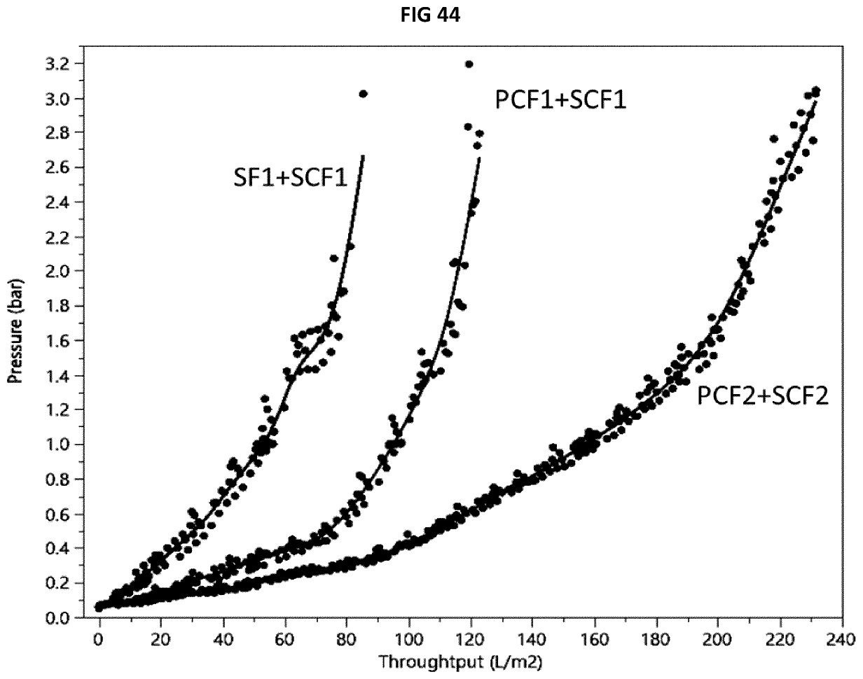

FIG. 44: Pressure monitoring for filters combinations SF1+SCF1, PCF1+SCF1 and PCF2+SCF2

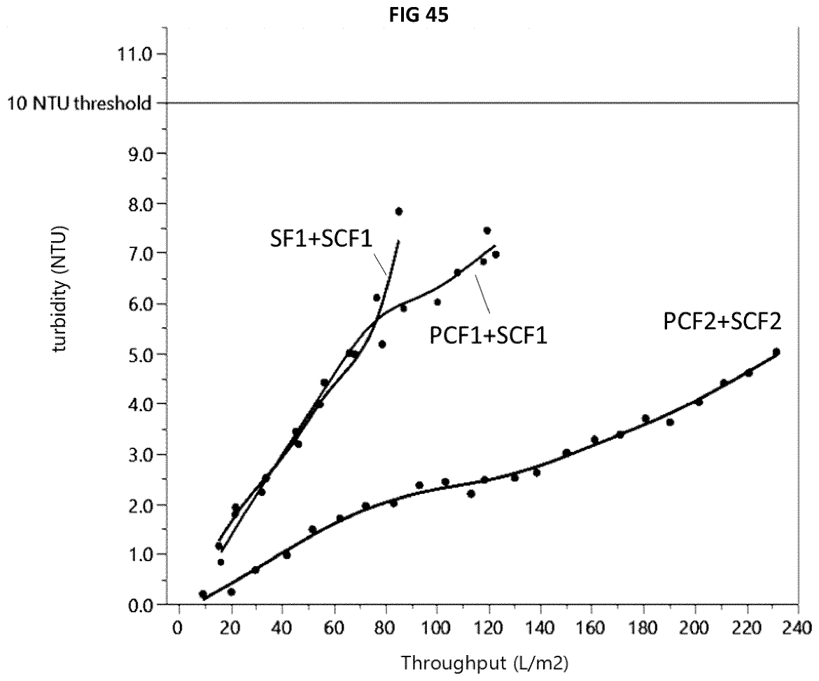

FIG. 45: Turbidity monitoring for filters combinations SF1+SCF1, PCF1+SCF1 and PCF2+SCF2

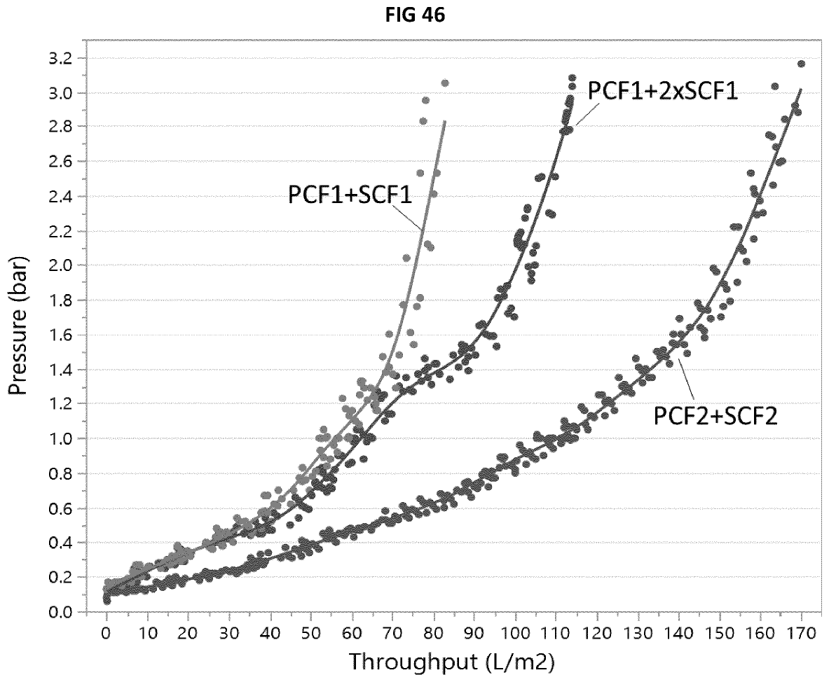

FIG. 46: Pressure monitoring for filters combinations PCF1+SCF1, PCF1+2×SCF1 and PCF2+SCF2

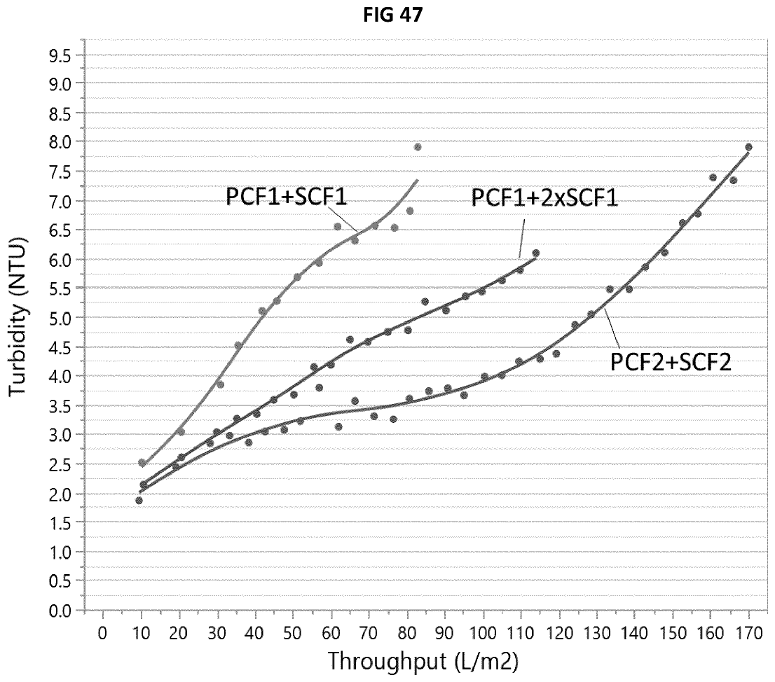

FIG. 47: Turbidity monitoring for filters combinations PCF1+SCF1, PCF1+2×SCF1 and PCF2+SCF2

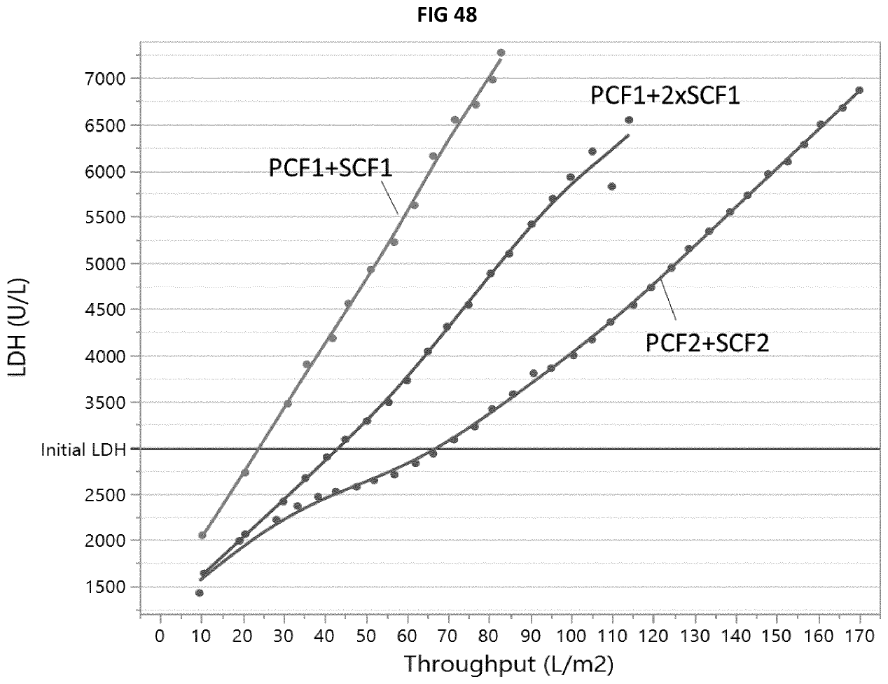

FIG. 48: LDH monitoring for filters combinations PCF1+SCF1, PCF1+2×SCF1 and PCF2+SCF2

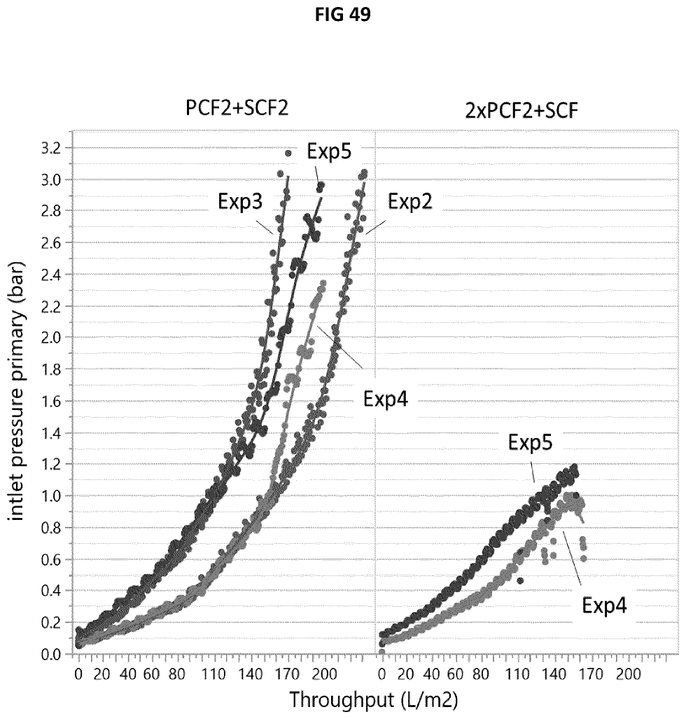

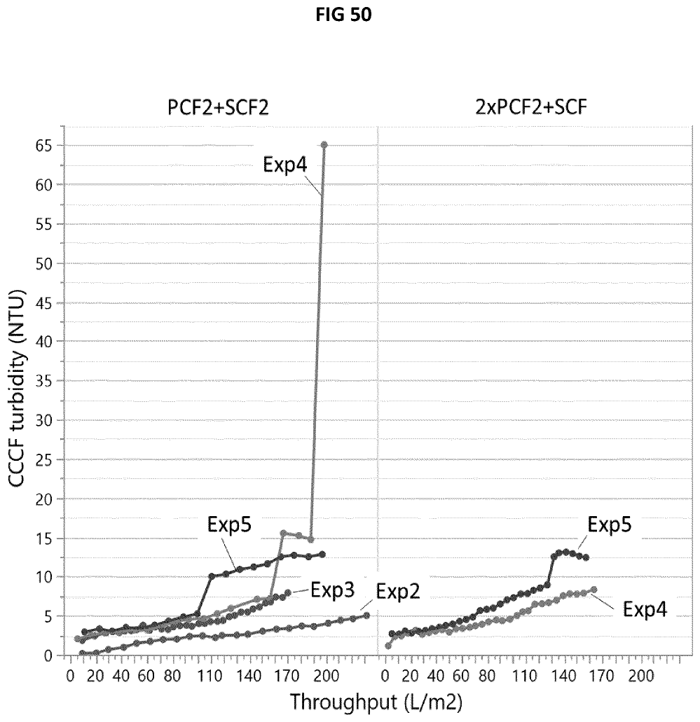

FIG. 49: Pressure comparison with CCF from best (Exp2), base (Exp3 and Exp4) and worst case (Exp5) experiments

FIG. 50: Turbidity comparison with CCF from best (Exp2), base (Exp3 and Exp4) and worst case (Exp5) experiments

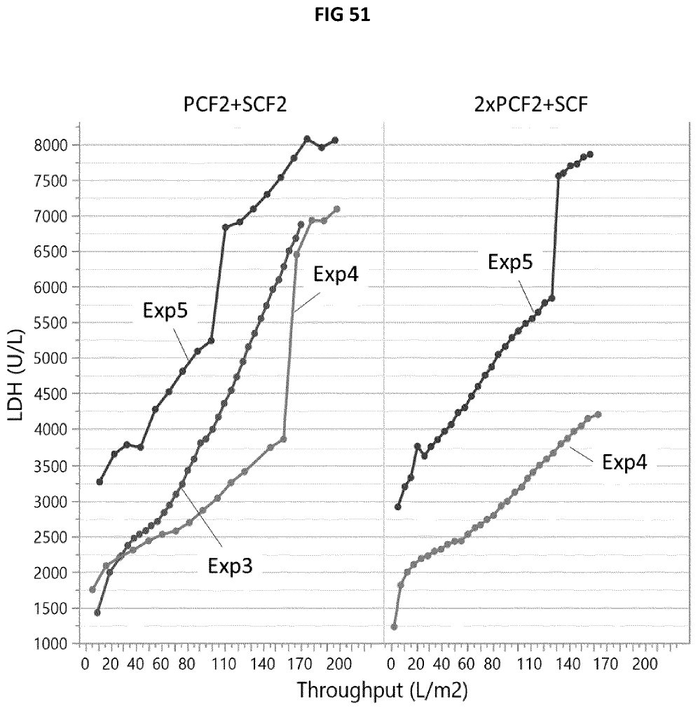

FIG. 51: LDH comparison with CCF from base (Exp3 and Exp4) and worst case (Exp5) experiments

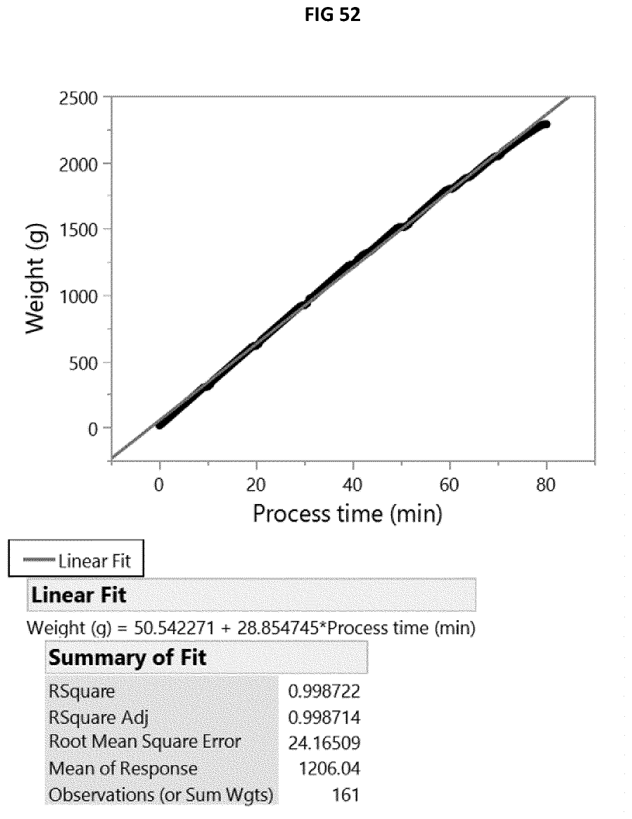

FIG. 52: Collected weight over the time to calculate the experimental flow in Exp6 trial

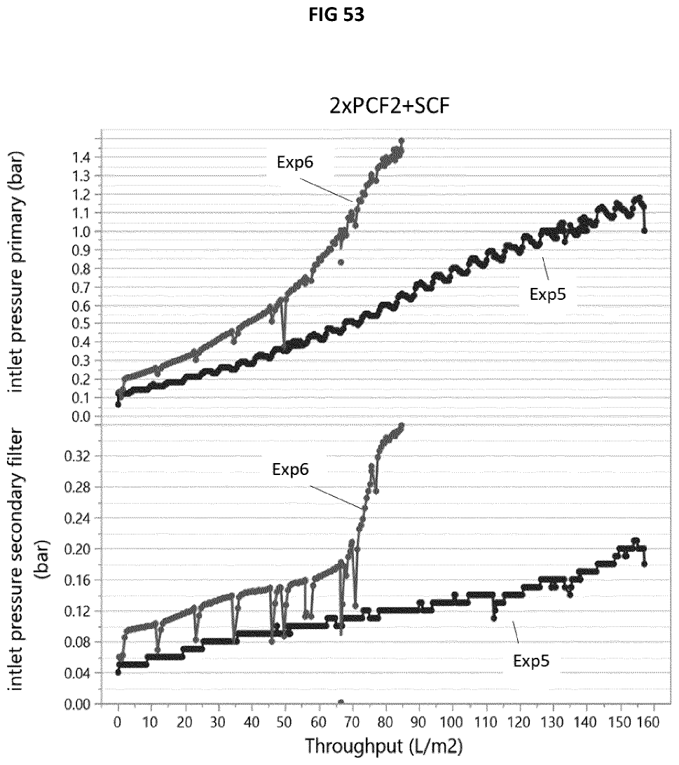

FIG. 53: Pressure comparison between small scale (Exp5) and bench scale (Exp6) trials

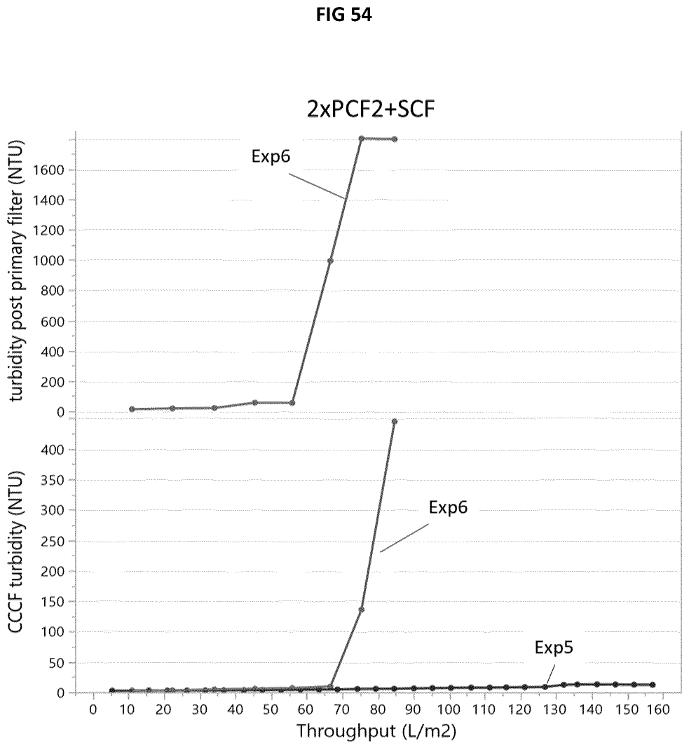

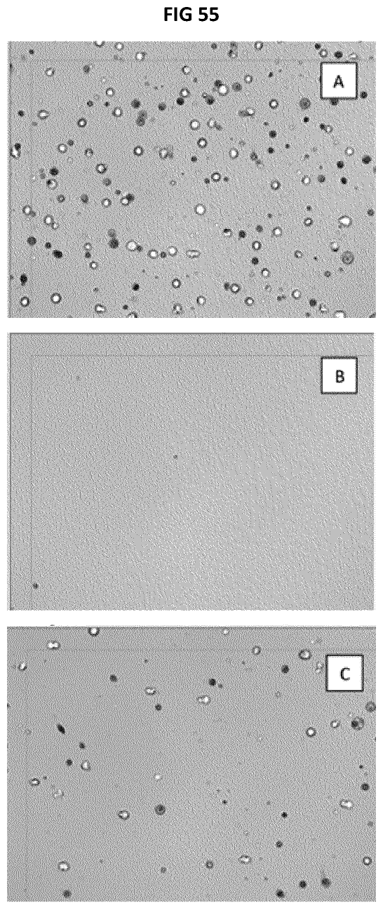

FIG. 54: Turbidity comparison between small scale (Exp5) and bench scale (Exp6) trials

FIG. 55: Vicell pictures at different states of the bench scale trial. A—Post PCF2 at 1 bar sampling, B—post SCF2 at 1 bar sampling, C—Post SCF2 sampling at the end of the trial

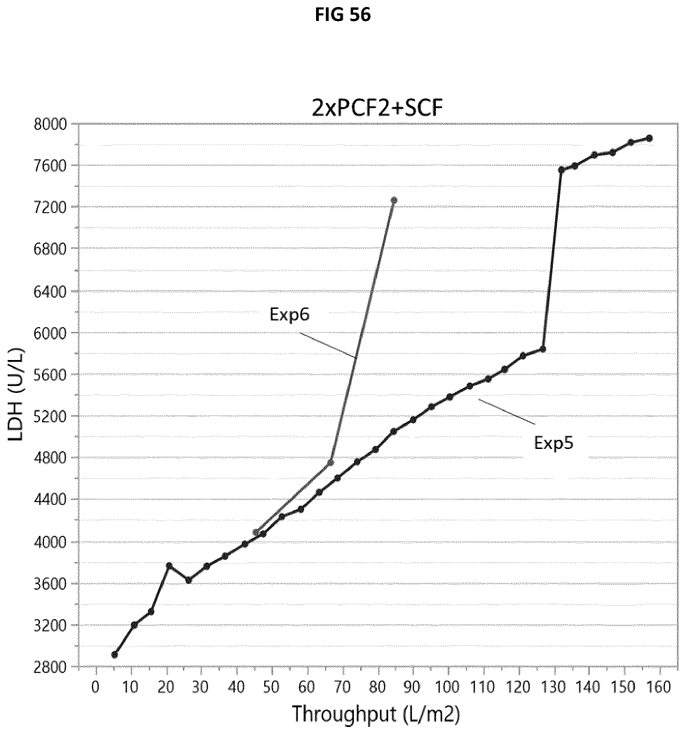

FIG. 56: LDH comparison between small scale (Exp5) and bench scale (Exp6) trials

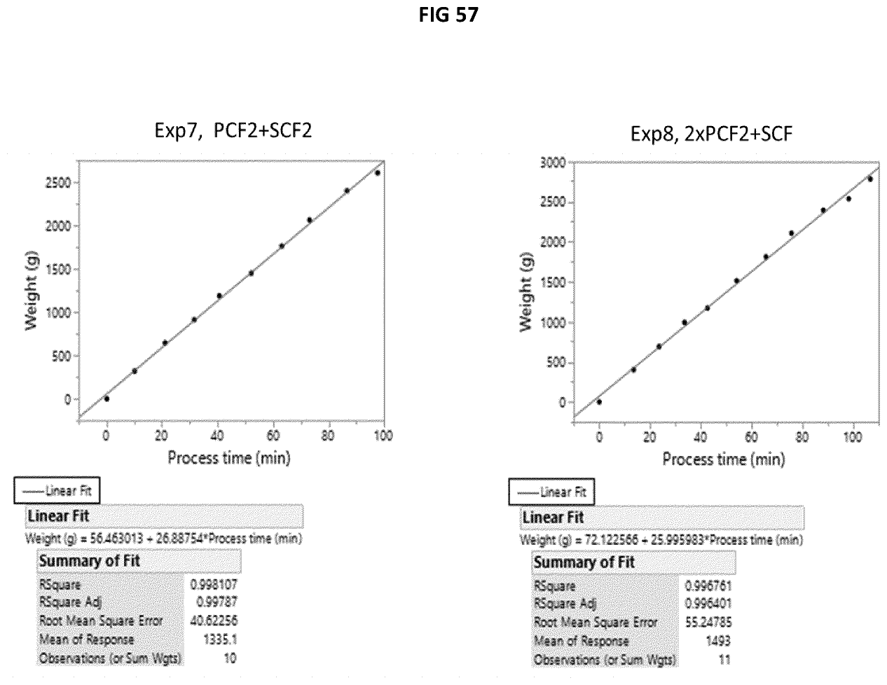

FIG. 57: Collected weight over the time to calculate the experimental flow in Exp7 and Exp8 trials

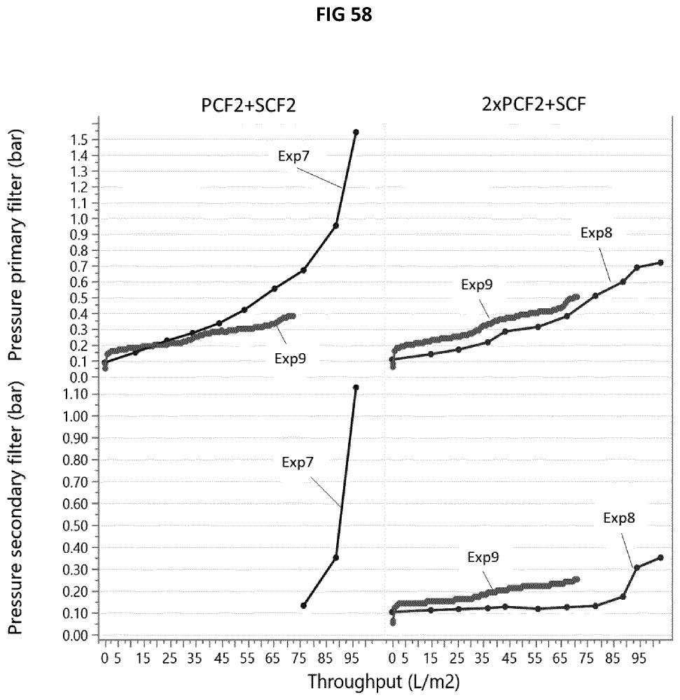

FIG. 58: Pressure comparison between small scale (Exp9) and bench scale (Exp7 and Exp8) trials

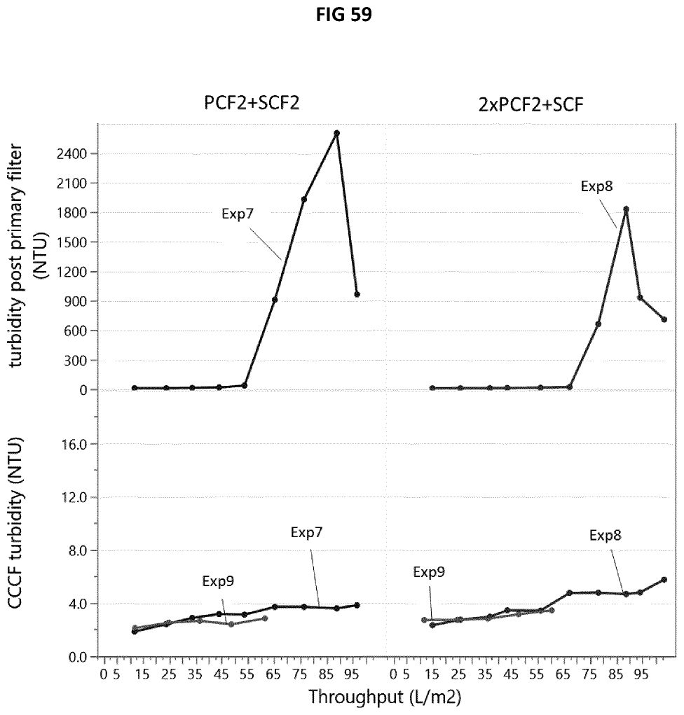

FIG. 59: Turbidity comparison between small scale (Exp9) and bench scale (Exp7 and Exp8) trials

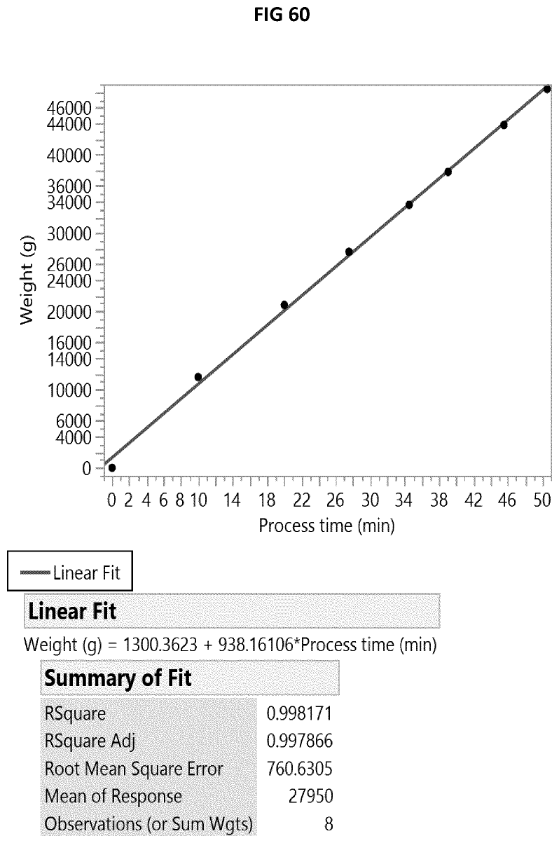

FIG. 60: Experimental flow of the pilot scale clarification

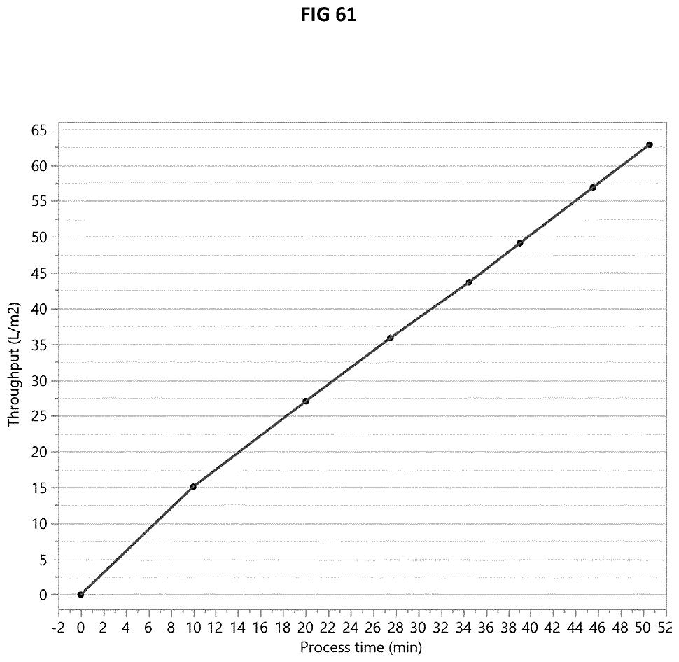

FIG. 61: Throughput of the pilot scale clarification

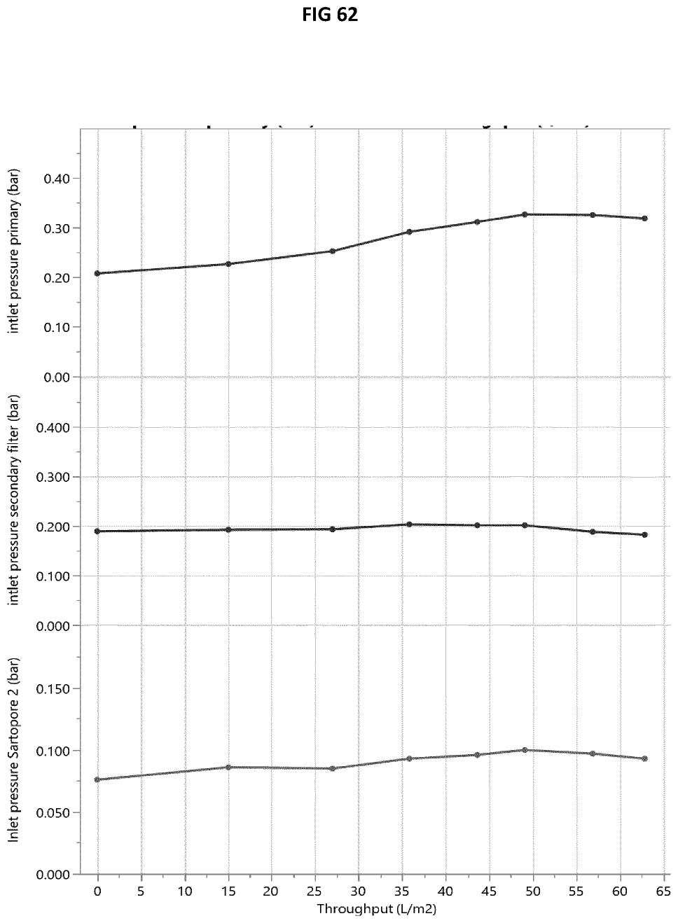

FIG. 62: Pressure of the pilot scale clarification

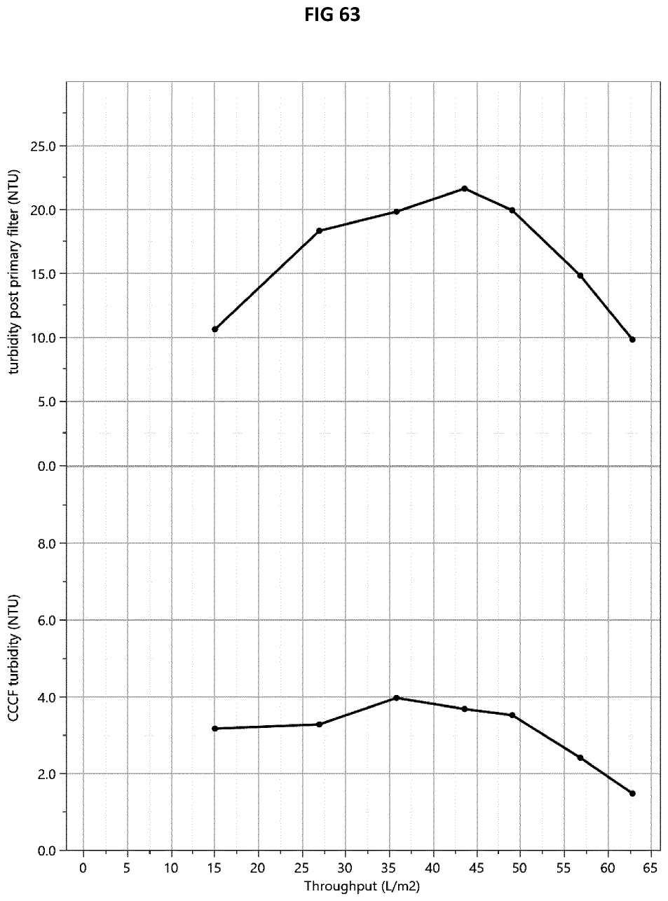

FIG. 63: Turbidity of the pilot scale clarification

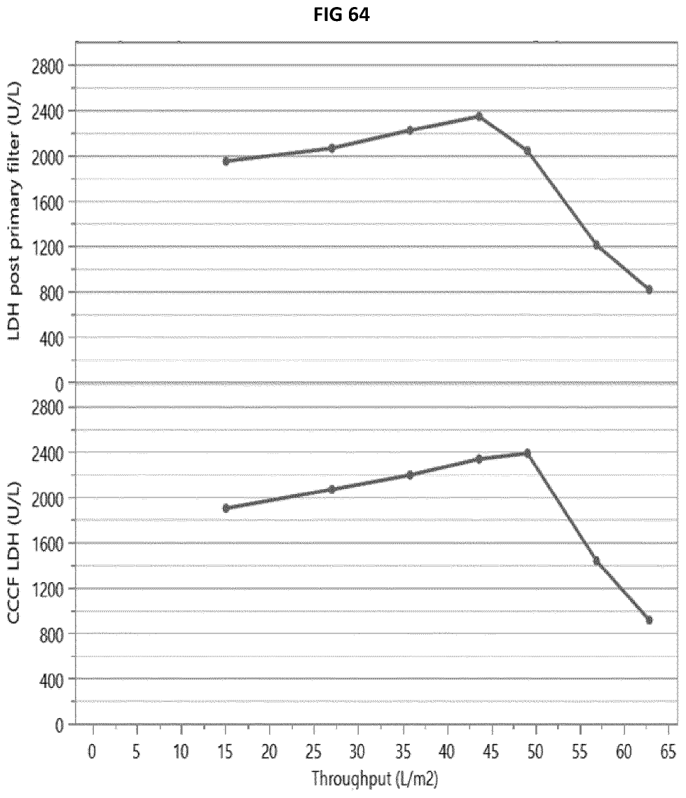

FIG. 64: LDH of the pilot scale clarification

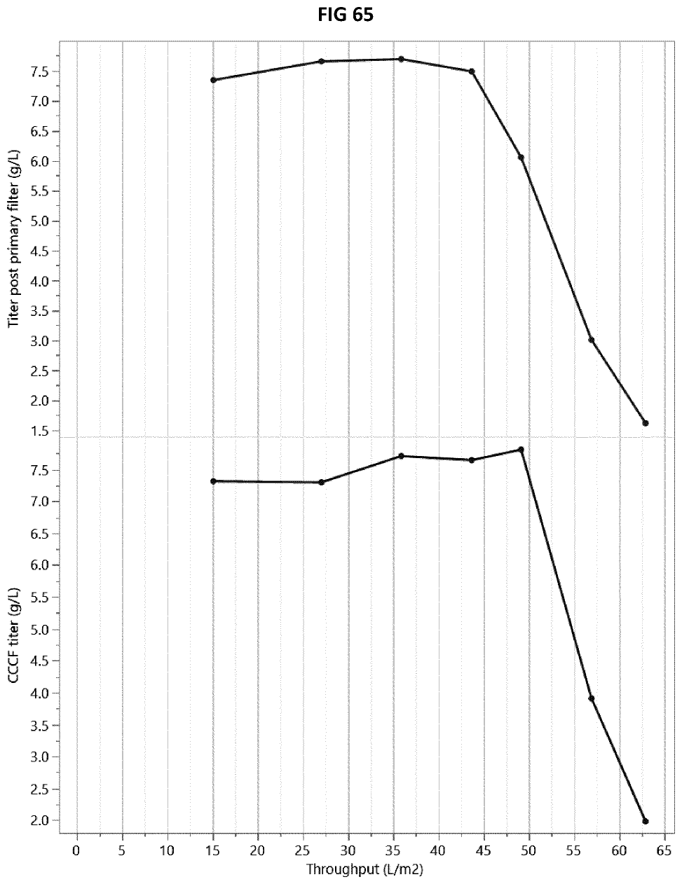

FIG. 65: CEDEX titer of pilot scale clarification

### EXAMPLE 1: MATERIAL AND METHODS

### Materials and Equipment for Cell Culture

The implementation of clarification experiments requires continuous cell culture fluid (CCF) generation in order to perform studies without interruption. CHO cell lines expressing antibodies with different physical and chemical properties were used in these study. Additional CCF was generated using 3 L bench scale bioreactors.

The characteristics of the cell lines and example of typical cell culture parameters after 14 days of culture are summarized in Table 1.

TABLE 1
Comparison cell lines

| | | Antibody | | | |
|---|---|---|---|---|---|
| | | expressed | VCC | | Antibody |
| Cell line | Cell | by the | (10⁶ c/ | Turbidity | titer |
| reference | line | cell line | mL) | (NTU) | (g/L) |
| A | CHO-1 | Ab1 | ~10-12 | ~1800-2100 | ~1-2 |
| B | CHO-1 | Ab2 | ~4-8 | ~1200-1400 | ~1.5-2.5 |
| C | CHO-2 | Ab3 | ~25-30 | ~2500-6000 | ~7-8 |

The assessment of the cell culture state requires the monitoring of different biological parameters such as cell density, viability and average cell diameter and also chemical parameters such as metabolite quantification or product of interest titer. For clarification the monitored parameters were: Viable Cell Concentration (VCC), cell culture viability, turbidity, titer and Lactate Dehydrogenase (LDH) at the end of the culture process. For CFF with low turbidity, such as turbidity lower than 3000 NTU, the cell viability was used as critical factor to classify the CCF the following categories: representative CCF (viability >75), challenging CCF (60<viability<75) and worst case CCF (viability <60%).

The cell counter Vi-Cell™XR (Beckman Coulter Ref 383536, USA) was used to automatically count the cells during the cell culture process on a desired basis depending of the importance of the culture. A sample of 500 μL from the cell culture was pumped into the analyzer and then automatically mixed with trypan blue in order to discriminate dead and viable cells. The resulting sample was imaged by the Vi-Cell software with a predefined method. The imaging algorithm can discriminate viable and dead cells based on their shape, size, brightness and color. This device allows to determine Total Cell density (TCC), Viable Cell density (VCC), viability and average cell diameter. It is also showing a cell size distribution graph as the Vi-cell is analyzing several images (50 or 100).

The Cedex Bio HT analyzer from Roche is a high-throughput automated metabolite analyzer based on enzymatic assays coupled with spectrophotometry which is able to monitor a wide range of various compounds of the cell broth such as glucose, glutamine, glutamate, lactate or ammonia. A 500 sample from the cell culture was centrifuged in order to separate the residual biomass from the supernatant. This device was used also to monitor the antibody titer in the cell broth. This assay is based on a turbidimetric method by precipitation of the IgG with an antiserum. This method allows a high comparability with the usual Protein A HPLC method. The LDH rate can be also monitored. This intracellular enzyme is involved in the degradation of carbon source. The cell membrane breakage triggers the release of this enzyme into the medium. Consequently, the assessment of this enzyme in the cell culture broth is a good marker to monitor the cell lysis rate.

### Set-Up for Clarification Process

The screening of numerous clarification alternatives required a set-up able to conduct different clarification experiments in parallel and to monitor process parameters such as pressure and throughput across the filter.

A Scilog FilterTec pump is a tri-headed pump allowing to run triplicate measurements from the same starting CCF or run three independent CCF in parallel at the same flow rate with three filters. Three balances were connected directly to the pump to get an automatic online reading of the weight over the time. Pressure monitoring was done by Scilog sensors. Sensors were directly connected to the pump also and online monitoring is recorded. The pump was connected to a computer able to monitor and record the online data, including pressure, weight and flow rate.

The turbidity was followed offline to detect any potential blockage or breakthrough. Cell lysis was assessed by LDH assay during the process. Retains were sampled at critical points in order to assess the antibody quality if required.

The turbidity is monitored by a turbidimeter from Hach® which measures the diffused light at 90° at a wavelength of 860 nm in Nephelometric Turbidity Unit (NTU). The turbidity is directly correlated with the haziness or cloudiness of a solution.

For the clarification process for a 50 L single use bioreactor (SUB), the targeted throughput is 50 L/m². Once this target is reached, additional CCF can be filtered in order to challenge the filter and reach its maximum capacity. The calculation of flow rates used for the small scale experiments is based on the 1 m² process with the SF1 membrane and adapted to the small filters area as described in Table 2 using a linear correlation for scale-down. Before filtration of the CCF, high purified water (HPW) flush is required to remove aqueous extractables and leachables from the filters such as metal ions or other inorganic component. A PBS flush is also needed to equilibrate the filter media in terms of pH and charge. A turbidity exceeding 20 NTU was considered to be a breakthrough in a primary clarification process and 10 NTU for a secondary clarification process. It was assumed that when the turbidity values exceed 20 NTU, some cells or cellular debris were crossing the primary depth filter membrane. However, the experiments were continued until the filter was fully blocked or a predefined pressure limit was reached. The CCCF (Clarified Cell Culture Fluid) before breakthrough is separated from the CCCF harvested after breakthrough in order to perform the secondary clarification with both, representative and worst-case CCCF. The 20 NTU threshold is expected to lead to challenging primary CCCF in order to test the limits of the secondary clarification process. It is assumed that the turbidity should not exceed 10 NTU at the end of the whole clarification process (primary and secondary clarification included) in order to avoid blocking of sterile filters placed at the end of the clarification step.

TABLE 2
Scale up based on LMH

| | Scale up based on LMH |
|---|---|
| Step | (L/m²/hour) |
| HPW WASH | 160 |
| PBS WASH | 160 |
| DEAD VOLUME | 100 |
| CLARIFICATION STEP | 70 |

### Depth Filtration and Clarification Alternatives

### Depth Filtration

To screen clarification by depth filtration, several filters were tested. The characteristics of the filters are described in detail in Table 3.

TABLE 3
Global description of depth filters,

| | | Exclusion | |
|---|---|---|---|
| Name | Media | range (μm) | Filter type |
| SF1 | Cellulose, resin and filters aids | 1-20 | Single step |
| | | | filter |
| SF2 | Cellulose, resin and filters aids | 0.2-2 | Single step |
| | | | filter |
| SF3 | High capacity synthetic media | 0.2-2 | Single step |
| | | | filter |
| PCF1 | Cellulose, resin and filters aids | 6-30 | Primary |
| | | | clarification |
| | | | filter |
| PCF2 | High capacity synthetic media | 0.55-8 | Primary |
| | | | clarification |
| | | | filter |
| PCF3 | Cellulose, resin and filters aids | 0.55-8 | Primary |
| | | | clarification |
| | | | filter |
| PCF4 | Cellulose, resin and filters aids | 10-1.5 | Primary |
| | | | clarification |
| | | | filter |
| PCF5 | Cellulose, resin and filters aids | 0.7-5 | Primary |
| | | | clarification |
| | | | filter |
| PCF6 | Cellulose, resin and filters aids | 0.25-5 | Primary |
| | | | clarification |
| | | | filter |
| SCF1 | Cellulose, resin and filters aids | 0.2-3.5 | Secondary |
| | | | clarification |
| | | | filter |
| SCF2 | High capacity synthetic media | <<0.1 | Secondary |
| | | | clarification |
| | | | filter |
| SCF3 | Cellulose, resin and filters aids | <<0.1 | Secondary |
| | | | clarification |
| | | | filter |
| SCF4 | Cellulose strongly charged resin | 0.1-3 | Secondary |
| | and filters aids | | clarification |
| | | | filter |
| SCF5 | Membrane absorber with quaternary | <<0.2 | Secondary |
| | ammonium resin | | clarification |
| | | | filter |

### Centrifugation

Two centrifuges were used for the experiments. For small scale experiments, an Eppendorf centrifuge 5810R was used. This centrifuge was able to process CCF in 15 mL or 50 mL tubes from 100 to 3000 G. This centrifuge was used for small scale centrifugation experiments with shake flask produced CCF. The Hermle Z513 centrifuge has a maximum capacity of 6×250 ml or 4×500 ml depending of the rotor used at maximum speed of 16000 G in order to treat larger scale volume of CCF produced in bioreactor.

### Flocculation

The polydiallyldimethylammonium chloride (pDADMAC) is a positively charged polymer that induces interaction between negative charged particle such as cells or cell debris to trigger the precipitation of the latter. The pDADMAC 10% weight solution used for the flocculation experiments has been provided by Merck Millipore. A preliminary dosing study was needed to determine the suitable quantity of pDADMAC to add to the feed solution. During this dosing study, six solutions with flocculation agent from 0 to 0.1% of the weight of cells were prepared. The lowest turbidity of the supernatant was then measured in order to determine the best dose of polymer to add to the cell broth as described in FIG. 1. The lowest turbidity is reached when the amount of pDADMAC is sufficient to trigger the aggregation of cell and cell debris. At higher polymer concentration the aggregation does not happen as the polymer tends to aggregate itself. Based on these results it was decided to add 9 mL of 10% pDADMAC solution to 3000 mL of initial CCF in order to reach 0.03% v/v, leading to the lowest turbidity.

### Acoustic Wave Separation

Acoustic wave separation (AWS) is a technology that exploits low frequency acoustic forces to generate a three dimensional standing wave across a flow channel. Cell culture from a fed batch bioreactor enters the flow channel, and as the cells pass through the 3D standing wave they are trapped by the acoustic forces. The trapped cells migrate to the nodes of the standing wave, and begin to clump together till such time as their buoyancy decreases and they settle out of the suspension by gravity. This allows a significant reduction in turbidity and reduces the area requirements for secondary clarification using depth filtration and subsequent filtration for bioburden control. AWS was performed using Cadence™ Acoustic Separator system from Pall, according to the manufacturer's instruction. Prior the testing of AWS in combination with depth filter, studies have been performed to optimize the AWS operational parameters (data not shown).

### Bioburden Reduction

In bench scale filtrations, bioburden reduction is performed by one reduction filter, which is a combination of a 0.45 μm layer and a 0.22 μm layer in a single cartridge (Sartopore 2). At pilot scale, two Sartopore 2 filters are used to avoid clogging the second one attached to the sterile bag.

### EXAMPLE 2: PRIMARY AND SECOND RECOVERY OF THE CLARIFICATION PROCESS BY A SINGLE DEPTH FILTER

First the ability of a clarification system composed of a SF1 depth filter is assessed. Following the depth filter, the bioburden reduction is done using two sterile filters as previously described. First, a scale down model was developed using 22 cm² of the SF1 depth filter media, this process was developed with a ratio of 50 L/m². As a result, the filtration goal was 110 mL for the 22 cm² surface.

### Process Robustness Assessment

The two different cell lines (A and B) cultivated in fed-batch mode were clarified with SF1 22 cm² depth filter at 14 and 17 days of culture. A summary of the conditions is described in Table 4.

TABLE 4
Description of the CCF used

| | Culture | VCC | | Initial | Initial | | |
|---|---|---|---|---|---|---|---|
| Cell | Duration | 10⁶ c/ | Viability | turbidity | LDH | CCF | Experimental |
| Line | (days) | mL | (%) | (NTU) | (U/L) | classification | conditions |
| A | 14 | 12.15 | 83.4 | 1883 | NA | Representative | Trial 1 |
| A | 17 | 10.31 | 69.4 | 2118 | NA | Challenging | Trial 2 |
| B | 14 | 4.58 | 71.9 | 1358 | 1108 | Challenging | Trial 1 |
| B | 14 | 7.54 | 86.2 | 1240 | 989 | Representative | Trial 2 |

As shown in FIG. 2 and FIG. 3, for cell line A at the targeted throughput of 50 L/m² the inlet pressure in the SF1 filter was 0.4 bar and the turbidity was 20 NTU which was the turbidity threshold to guarantee the performance of the following step. The differences between the two experiments with cell line A CCF were visible at higher throughput such as at 75 L/m² with 0.9 bar for the 17 days culture duration CCF whereas the pressure was only 0.5 bar for the 14 days culture length CCF. In the same way, at 75 L/m² the turbidity for trial 1 was 30 NTU and 40 NTU for trial 2. Thus, as expected the culture duration showed an impact on the clarification process as a 3 days older culture has a lower viability of 14%, which induces a 55% increase of filter pressure and 25% of filtrate turbidity with the SF1 filter.

For cell line B, at the targeted throughput of 50 L/m² the clarification went well with a low inlet pressure of 0.1 bar and a low turbidity of 3 NTU. After this target no difference was observed in terms of turbidity since 120 L/m² was reached. At this value, the monitored pressure is 1.50 bar with a filtrate turbidity of 50 NTU for trial 1 and only 0.5 bar with a filtrate turbidity of 30 NTU for trial 2. This difference can be explained by a faster filter fouling with trial 1 CCF which had a viability of 71.9% versus 86.2% for trial 2 CCF.

To conclude, two different behaviors can be observed in this experiment, where CCF from cell line A with higher cell density and turbidity than cell line B showed a faster tendency to breakthrough. As CCF A provided a larger amount of particles especially whole cells and cell debris than CCF B, it induced a faster fouling of the filter with pressure increase leading to breakthrough. With these two different CCFs, we could determine that for cell line A, the SF1 limit was 50 L/m² whereas it can be safely extended to 100 L/m² for cell line B.

Identification of Process Limitations with Worst Case CCF Testing

A study with thawed CCF was then conducted. That constituted a worst-case study CCF in terms of viability as the freezing leads to cell death and loss of cell integrity. Consequently, the CCF was expected to have a large amount of debris that would likely clog the filter. This CCF can be considered a worst case. This study was conducted in order to predict and understand SF1 behavior in worst case conditions to determine further process limitations.

TABLE 5
Description of the CCF used

| | Length of | VCC | | Initial | Initial | |
|---|---|---|---|---|---|---|
| Cell | culture | (10⁶ c/ | Viability | turbidity | LDH | Experimental |
| Line | (days) | mL) | % | (NTU) | (U/L) | conditions |
| A | 14 | 1.71 | 25 | 3380 | NA | Thawed CCF |

In FIG. 4 and FIG. 5, thawed CCF showed a major breakthrough issue since the beginning of the process at only 15 L/m² noticeable in the increase of the pressure in the filter and offline turbidity. At the targeted throughput of 50 L/m² the pressure with the thawed CCF was at 1.65 bar and the filtrate solution has a turbidity of 190 NTU compared with the 0.4 bar pressure and the 20 NTU turbidity with the cell line A representative CCF.

The throughput was calculated by monitoring the filtrate weight over time (FIG. 6). The same throughput trend was observed for the representative cell line A CCF meaning that the fouling of the filter was not sufficient to compromise its internal flow. But thawed CCF showed a decrease of the throughput over time meaning that the filter fouling is sufficient to cause an increasing blockage of the filter. This could be due to the creation of a massive filtrate cake that prevented the stream.

In conclusion, SF1 works well when the initial CCF has a viability higher than 70%. Nevertheless, SF1 shows limitations in the case of biomass accumulation followed by a viability drop. This case leads to SF1 clogging issue and breakthrough. To optimize the clarification process, alternative to the current platform have been investigated as follows.

### EXAMPLE 3: PRIMARY AND SECOND RECOVERY OF THE CLARIFICATION PROCESS BY A SINGLE DEPTH FILTER OR BY THE COMBINATION OF TWO OR MORE DEPTH FILTERS

First other single depth filters or the combination of two or more depth filters was tested.

### Screening of Depth Filter Alternatives

Comparison studies were performed at small scale with different filters. The performance of these filters was evaluated from the clarification performance of SF1 with a ratio of 50 L/m². Experimental conditions included primary clarification with large removal rate filters designed to remove large particle followed by secondary filtration with tight (narrow pores) filters.

### Characterization of the Depth Filters PCF1 and SCF1

This study was based on representative CCF from cell line B, produced in fed-batch condition in 2.5 L Thomson harvested at day 14. This culture was considered to be representative for CCF in the platform processes and is described in Table 6. This experiment assessed the performance of filters PCF1 and SCF1 compared with SF1 current platform (see Table 3 for a detailed description of the filters).

TABLE 6
Description of the representative CCF

| | Length of | TCC | VCC | | Initial | Initial |
|---|---|---|---|---|---|---|
| Cell | culture | (10⁶ c/ | (10⁶ c/ | Viability | turbidity | LDH |
| Line | (days) | mL) | mL) | (%) | (NTU) | (U/L) |
| B | 14 | 8.75 | 7.54 | 86.2 | 1240 | 898 |

FIG. 7 and FIG. 8 show that at the targeted throughput of 50 L/m² SF1 filter reaches a higher pressure of 0.15 bar whereas it was at 0.02 bar for the two PCF1 filters. Beyond this threshold the SF1 showed a rapid tendency to clog with a rapid increase of pressure and a maximum pressure of 1.6 bar reached at 175 L/m². A slower increase was observed for the two PCF1. At 200 L/m² PCF1 still showed an acceptable pressure of 0.2 bar. The maximum loading capacity of SF1 filter with this CCF corresponding to the inflexion point of turbidity vs throughput curve was determined to be 75 L/m².

Concerning the turbidity of the filtrate, the PCF1 CCCF showed a higher turbidity right from the beginning of test at 15 NTU compared to 2 NTU for SF1. During the clarification at 150 L/m² the filtrate turbidity of SF1 showed an increase and reached 80 NTU indicating that this filter was facing breakthrough issues whereas PCF1 filtrate turbidity was quite stable around 50 NTU with only a slight increase during the study. For PCF1 the maximum loading capacity appeared to be at 235 L/m² when a major breakthrough, characterized by the inflexion of turbidity (NTU) versus throughput (L/m²) curve, appeared with filtrate turbidity around 1000 NTU at the end of the test.

PCF1 filter had wider pores than SF1, which led to a lower tendency to clog but a higher filtrate turbidity in normal conditions (no breakthrough). Both PCF1 duplicates show the same behavior reflecting the equivalent performance of these filters.

Then, primary CCCF with SF1 and PCF1 were pooled in two samples to make a best case primary CCCF and a challenging CCCF to be clarified with the secondary clarification filter SCF1. The best-case CCCF had a turbidity of 24 NTU (SCF1) and challenging CCCF had a turbidity of 118 NTU (SCF1_challenging CCF).

Two different behaviors were observed in FIG. 9 and FIG. 10. At the targeted throughput, the filters pressure remained the same around 0.2 bar. Nevertheless, a pressure increase is observed for challenging CCCF with 2.6 bar of pressure at the end of the test at 200 L/m² against 0.41 bar for best case CCCF.

Despite this high pressure difference, no breakthrough was observed as filtrate turbidity remained the same for both filter during the clarification at around 3 NTU showing that PDPE2 filters are resistant filters not sensitive to breakthrough.

The results of this study with representative CCF are described in Table 7 below.

TABLE 7
Summary of clarification results

| | | Pressure at | Turbidity at |
|---|---|---|---|
| | Maximum | maximum | maximum |
| | loading | loading | loading |
| Depth | capacity | capacity | capacity |
| filter | (L/m²) | (bar) | (NTU) |
| SF1 | 115 | 0.6 | 20 |
| PCF1 | 235 | 0.3 | 70 |
| SCF1 | 240 | 0.4 | 2 |
| SCF1 | 200 | 2.6 | 2 |
| challenging | | | |
| CCCF | | | |

The PCF1 filter was not designed to work as a single filter as its filtrate turbidity was high right from the beginning of the clarification. The SCF1 filter seems to be designed to tolerate a pre clarified CCF with a high turbidity such as the output of the PCF1 filter with a representative CCF.

Characterization of the depth filters SF2, SF3, PCF2, PCF3, SCF2 and SCF3 This study was based on two representative CCF produced in fed-batch condition in 2.5 L Thomson harvested at day 14 from cell line B. This CCF was considered as being representative for the platform process and is described in Table 8 below. Different filter types were evaluated in this study, including filters that can be used for primary and secondary clarification, filters that are suitable just for primary clarification or just for secondary clarification filters. These filters were small scale filters of 23 cm² (see Table 3 for detailed filter descriptions).

TABLE 8
Description of the representative case CCF

| | Culture | TCC | VCC | | Initial | Initial | |
|---|---|---|---|---|---|---|---|
| Cell | duration | (10⁶ c/ | (10⁶ c/ | Viability | turbidity | LDH | Experiment |
| Line | (days) | mL) | mL) | (%) | (NTU) | (U/L) | ID |
| B | 14 | 5.10 | 4.15 | 81.3 | 1202 | 1039 | trial 1 |
| B | 14 | 4.92 | 3.82 | 77.7 | 1194 | 1114 | trial 2 |

During the process the feed flow was progressively increased from the current flow in order to challenge the filter capacity. As described in FIG. 11 and FIG. 12 at the targeted throughput of 50 L/m² turbidity remained low around 5 NTU for all filters with the two different CCFs except the PCF3 which showed a filtrate turbidity of 25 NTU. The PCF2 and PCF3 filters pressure remained near 0. Filters with narrow pores such as SF2 and SF3 exhibited a pressure between 0.1 and 0.15 bar. The pressure increase was correlated with filter porosity. Filters with wide pores such PCF2 and PCF3 showed a slight increase of the pressure whereas filters with tight pores (SF2 and SF3) showed a higher pressure increase. The filter media composition, especially inorganic filters such as SF3 make them less vulnerable to clogging as pressure increase is less important than for SF2. The same behavior is observed for PCF2 and PCF3 filters. During the filtration process, synthetic media filters showed almost no sign of breakthrough. Based on this experiment, the best filter for primary clarification was PCF3 with a maximum loading capacity of 400 L/m². Between the two experiments this filter exhibited no significant behavior differences meaning this filter was able to handle at least small differences in the initial CCF.

A small difference was observed between the SF2 filters as the maximum loading capacity (corresponding to inflexion point of the turbidity vs throughput curve) for trial 1 was at 125 L/m² and 150 L/m² for trial 2 indicating that SF2 filters may be affected by the minor differences in the initial CCF (3.5% in viability and 0.5×10⁶ c/mL in VCC).

PCF2 shows better performance than PCF3 due to its synthetic composition whereas PCF3 has organic composition. Moreover, PCF2 has 4 layers of membrane inside the filter whereas PCF3 has only 2.

Primary CCCF with PCF3 and SF3 filters was chosen for the secondary filtration with filters having narrow pores (SCF2 and SCF3) as described in FIG. 13 and FIG. 14. This CCCF had an initial turbidity of 31.8 NTU.

No difference was observed at 50 L/m² in terms of pressure or turbidity between SCF3 and SCF2 filters. Both filters exhibited a filtrate turbidity around 2 NTU and a pressure of 0.2 bar. In terms of turbidity no difference was observed during the study meaning that no breakthrough happened during this experiment, even when challenged at high flow rates. Nevertheless, at 200 L/m² SCF3 showed some filter clogging with an increase in pressure up to 3 bars whereas the SCF2 filter at the same throughput had a pressure of 1 bar. As the filters have the same exclusion range it showed that SCF2 had a better filter capacity with a final calculated loading capacity of 350 L/m², compared to 200 L/m² for the SCF3. The SCF2 filter was then selected as the best secondary filter in this case.

Results are summarized in Table 9, the turbidity values in this table correspond to the maximum loading capacity.

TABLE 9
Clarification results summary

| | | Pressure at | Turbidity at |
|---|---|---|---|
| | Maximum | maximum | maximum |
| | loading | loading | loading |
| Depth | capacity | capacity | capacity |
| filter | (L/m²) | (bar) | (NTU) |
| SF2 trial 1 | 125 | 1.2 | 22 |
| SF2 trial 2 | 140 | 1.6 | 20 |
| SF3 trial 2 | 300 | 1.98 | 10 |
| PCF2 trial 1 | 325 | 0.4 | 16 |
| PCF2 trial 2 | 400 | 0.79 | 20 |
| PCF3 trial 1 | 75 | 0.2 | 29 |
| SCF2 trial 2 | 350 | 2.2 | 2 |
| SCF3 trial 2 | 200 | 2.8 | 2 |

### Characterization of the Depth Filters PCF4, PCF5, PCF6, SCF4 and SCF5

A representative CCF was generated in a 14 days fed-batch with the characteristics described in Table 10. The filters studied were the primary clarification filters PCF4, PCF5 and PCF6 followed by the secondary clarification filters SCF5 and SCF4 (see Table 3 for a detailed filter description).

TABLE 10
Description of the representative CCF

| | Length of | TCC | VCC | | Initial | Initial |
|---|---|---|---|---|---|---|
| Cell | culture | (10⁶ c/ | (10⁶ c/ | Viability | turbidity | LDH |
| Line | (days) | mL) | mL) | (%) | (NTU) | (U/L) |
| B | 14 | 7.18 | 6.11 | 85.1 | 1202 | 1211 |

In FIG. 15 and FIG. 16, at 50 L/m² depth filters showed the same behavior in terms of inlet pressure with a pressure around 0.1 bar. On the other hand two different behaviours can be observed by comparing filtrate turbidity of these filters. Filters with larger pores like PCF4 or PCF5 (refer to Table 3 for ranges) had a higher filtrate turbidity of around 20 NTU than filters with narrow pores like PCF6 which have a filtrate turbidity at 5 NTU. Breakthrough appeared at the same throughput of 80 L/m² for PCF4 and PCF5 at pressures, of 0.12 and 0.22 bar, respectively. The tight filter PCF6 showed a breakthrough at 0.50 bar for a throughput of 140 L/m².

Primary CCCF without breakthrough was pooled in order to test the secondary clarification depth filters SCF4 and SCF5. The primary CCCF obtained had an initial turbidity of 30.3 NTU.

At 50 L/m² SCF4 showed a higher pressure of 0.20 bar than the SCF5 which was at 0.05 bar. Filtrate turbidity remained the same for both filters during the experiment with a value around 2 NTU as described in FIG. 17 and FIG. 18.

The SCF5 filter showed the best filtration performance as the pressure remains stable over the time whereas it increased faster for the SCF4 filter.

The filter performances are summarized in Table 11.

TABLE 11
Clarification results summary

| | | | Turbidity at |
|---|---|---|---|
| | Maximum | | maximum |
| | loading | Maximum | loading |
| Depth | capacity | pressure | capacity |
| filter | (L/m²) | (bar) | (NTU) |
| PCF4 | 80 | 0.2 | 30 |
| PCF5 | 80 | 0.2 | 30 |
| PCF6 | 130 | 0.5 | 20 |
| SCF4* | 115 | 0.3 | 2 |
| SCF5* | 115 | 0.05 | 2 |

*Trials stopped because of the lack of the of initial material

### Characterization of Depth Filters Combinations

Based on the results reported above, the filters showing the best clarification performances are PCF1 and SCF1 and PCF2 and SCF2, therefore their combination was tested.

Representative CCF cultivated in Shake Flasks in fed-batch mode for 14 days was used to test the following combinations of filters: PCF1+SCF1 and PCF2+SCF2; study description reported in Table 12.

TABLE 12
Description of the representative CCF used
in depth filters combinations study

| | Length of | TCC | VCC | | Initial | Initial |
|---|---|---|---|---|---|---|
| Cell | culture | (10⁶ c/ | (10⁶ c/ | Viability | turbidity | LDH |
| Line | (days) | mL) | mL) | (%) | (NTU) | (U/L) |
| B | 14 | 6.69 | 5.72 | 85.5 | 1184 | 1119 |

Clarification was performed with a tri-headed pump from the same representative CCF in order to compare primary filters based on the same parameters. The same surface of primary and secondary filters had been chosen in order to have comparable performance. PCF3 showed slow pressure increase with 2 bar reached at 600 L/m² compared to the PCF1 which reached 2 bar at 225 L/m².

As shown in FIG. 19, the pressure increase in the primary filters was rapidly followed by secondary filter. At 225 L/m² SCF1 reached 1.6 bar and at 600 L/m² SCF2 reached 1 bar. Due to pressure increase PCF1+SCF1 combination has to be stopped earlier than PCF2+SCF2 combination. PCF2+SCF2 depth filters combination appeared to be the best depth filtration solution in terms of pressure increase with a loading capacity three time more than PCF1+SCF1. The clogging observed in secondary filters was certainly due to primary filter breakthrough.

As shown in FIG. 20, the results of the assessment of the clarification performance (turbidity) with representative CCF, indicate that none of the filters combination showed breakthrough, and turbidity remained below 5 NTU. PCF2+SCF2 combination showed an overall filtrate turbidity inferior of 0.5 NTU compared to PCF1+SCF1 filtrate turbidity. Also, despite filters clogging issue, none of the secondary filters exhibited breakthrough.

In conclusion, PCF2+SCF2 combination exhibits the best clarification performance compared to the PCF1+SCF1 combination with final throughput reached at 600 L/m² compared to the 235 L/m² reached with the PCF1+SCF1, at pressure equal to 3 bars (see summary table Table 13). Both the 1 bar pressure, indicated as limit by the manufacturer and the 3 bar pressure used as superior worse case limit were investigated.

TABLE 13
Clarification results summary for depth filters combinations study

| | | | Pressure at | Turbidity at |
|---|---|---|---|---|
| | | Maximum | maximum | maximum |
| | Initial | loading | loading | loading |
| | turbidity | capacity | capacity | capacity |
| Filter | (NTU) | (L/m²) | (bar) | (NTU) |
| PCF1 + SCF1 | 1184 | 125 | 1.0 | 2.0 |
| PCF2 + SCF2 | 1184 | 450 | 1.0 | 3.1 |
| PCF1 + SCF1 | 1184 | 250 | 3.0 | 2.5 |
| PCF2 + SCF2 | 1184 | 600 | 3.0 | 3.5 |

Characterization of Depth Filters with Challenging CCF from Bioreactors

A challenging CFF with low viability was generated in fed batch mode during 14 days in order to challenge filters and confirm the previous screening results. This CCF is described in Table 14. The depth filters studied for this characterization: SF2, SF3, PCF2, PCF3, SCF2 and SCF3.

TABLE 14
Description of the challenging CCF

| | Length of | TCC | VCC | | Initial | Initial | |
|---|---|---|---|---|---|---|---|
| Cell | culture | (10⁶ c/ | (10⁶ c/ | Viability | turbidity | LDH | |
| Line | (days) | mL) | mL) | (%) | (NTU) | (U/L) | Experiment |
| B | 14 | 12.14 | 7.89 | 65 | 2410 | 3420 | Trial 3 |

The study was performed at different LMH as the filtration surface was different, so filter resistance (bar/LMH) was plotted instead of pressure only. Primary clarification depth filter like PCF2 (or PCF3) and secondary ones like SCF3 (or SCF2) were directly combined in series with a ratio of 2:1 as recommended by the supplier.

At 50 L/m² as described in FIG. 21 and FIG. 22, SF2 and PCF3+SCF3 showed the same high pressure at 1 bar whereas SF3 and PCF2+SCF2 and SF3 exhibited the normal pressure of 0.1 bar. At this loading capacity SF3 and PCF2+SCF2 filters showed no sign of breakthrough with a turbidity of 10 NTU and 3 NTU, respectively. The PCF3+SCF3 filter combination showed no sign of breakthrough with 5 NTU of filtrate turbidity despite the observed high pressure. Breakthrough occurred in the SF2 filter with a filtrate turbidity of 50 NTU.

The PCF3+SCF3 combination had to be stopped at 70 L/m² as the pressure reached was at the filter limit of 2.5 bar. The SF3 showed better performance with a slow pressure increase with filtrate turbidity of 14 NTU at 90 L/m². The PCF2+SCF2 filter showed the best performance with a clarification of 205 L/m² and a turbidity of 12 NTU. The combination of PCF2+SCF2 filter appeared to be the best filtration option as seen in the summary Table 15.

TABLE 15
Clarification results summary

| | | Pressure at | Turbidity at |
|---|---|---|---|
| | Maximum | maximum | maximum |
| | loading | loading | loading |
| Depth | capacity | capacity | capacity |
| filter | (L/m²) | (bar) | (NTU) |
| SF2 trial 3 | 38 | 1.2 | 21.5 |
| SF3 trial 3 | 90 | 0.3 | 14.0 |
| PCF3 + | 70 | 2.5 | 4.2 |
| SCF3 trial 3 | | | |
| PCF2 + | 205 | 1.7 | 12 |
| SCF2 trial 3 | | | |

### Global Comparison of Depth Filters

The screening of depth filters allowed the identification of interesting alternatives to the current platform filter. The results of the tested filters are summarized in Table 16 below. The maximum throughput was determined according to standard criteria previously defined (<20 NTU for primary clarification and <10 NTU for secondary clarification). When these values were not reached, the experiment was continued until the pressure reached the value given as limit by the supplier. Pressure and turbidity were the value read when the maximum was reached before breakthrough.

In FIG. 23 and FIG. 24 primary clarification filters (and filters for primary and secondary recovery) are compared in order to determine the filter with the best performance. This graph demonstrates that PCF2 filter exhibited the best performance among others filters with a maximum throughput of 400 L/m² for the lowest filter resistance at 0.03 bar/LMH (0.35 bar). The comparison between all filter is summarized in FIG. 25.

TABLE 16
Summary of clarification performance

| | | | Pressure at | Turbidity at | | % of |
|---|---|---|---|---|---|---|
| | | Maximum | maximum | maximum | | improvement |
| | Initial | loading | loading | loading | % | relative to |
| | turbidity | capacity | capacity | capacity | Turbidity | SF1 |
| Filter | (NTU) | (L/m²) | (bar) | (NTU) | reduction | (100%) |

Single filters

| | | | | | | |
|---|---|---|---|---|---|---|
| SF1 | 1240 | 115 | 0.6 | 20 | 98.4 | 100 |
| SF2 trial 1 | 1202 | 125 | 1.2 | 22 | 98.2 | 109 |
| SF2 trial 2 | 1194 | 140 | 1.6 | 20 | 98.3 | 122 |
| SF2 trial 3 | 2410 | 63 | 1.2 | 21.5 | 99.1 | No |
| | | | | | | improvement |
| SF3 trial 2 | 1194 | 300 | 1.98 | 10 | 99.2 | 261 |
| SF3 trial 3 | 2410 | 128 | 0.3 | 14.0 | 99.4 | No |
| | | | | | | improvement |

Filters for primary clarification

| | | | | | | |
|---|---|---|---|---|---|---|
| PCF1 | 1240 | 235 | 0.3 | 70 | 94.4 | 204 |
| PCF2 trial 1 | 1202 | 325 | 0.4 | 16 | 98.7 | 283 |
| PCF2 trial 2 | 1194 | 400 | 0.79 | 20 | 98.3 | 348 |
| PCF3 trial 1 | 1202 | 75 | 0.2 | 29 | 97.6 | No |
| | | | | | | improvement |
| PCF4 | 1202 | 80 | 0.2 | 30 | 97.5 | No |
| | | | | | | improvement |
| PCF5 | 1202 | 80 | 0.2 | 30 | 97.5 | No |
| | | | | | | improvement |
| PCF6 | 1202 | 130 | 0.5 | 20 | 98.3 | 113 |

Filters for secondary clarification

| | | | | | | |
|---|---|---|---|---|---|---|
| SCF1 | 24 | 240 | 0.40 | 2 | 91.7 | 209 |
| SCF2 trial 2 | 31.8 | 350 | 2.2 | 2 | 93.7 | 304 |
| SCF3 trial 2 | 31.8 | 200 | 2.8 | 2 | 93.7 | 174 |
| SCF4 | 30.3 | 115 | 0.3 | 2 | 93.4 | No |
| | | | | | | improvement |
| SCF5 | 30.3 | 115 | 0.05 | 2 | 93.4 | No |
| | | | | | | improvement |

Combination of filters

| | | | | | | |
|---|---|---|---|---|---|---|
| PCF1 + | 1184 | 125 | 1.0 | 2 | 99.8 | 109 |
| SCF1 | | | | | | |
| PCF1 + | 1184 | 250 | 3.0 | 2.5 | 99.8 | 217 |
| SCF1 | | | | | | |
| PCF2 + | 1184 | 450 | 1.0 | 3.1 | 99.7 | 391 |
| SCF2 | | | | | | |
| PCF2 + | 1184 | 600 | 3.0 | 3.5 | 99.7 | 522 |
| SCF2 | | | | | | |
| PCF2 + | 2410 | 205 | 1.7 | 12 | 99.5 | 178 |
| SCF2 trial 3 | | | | | | |
| PCF3 + | 2410 | 70 | 2.4 | 4.2 | 99.8 | No |
| SCF3 trial 3 | | | | | | improvement |

PCF1 showed also good results with maximum throughput of 235 L/m² for a higher pressure at 0.005 bar/LMH (0.8 bar). Finally, the PCF6 filter showed a slight improvement toward SF1 with a maximum loading capacity of 130 L/m² for a pressure of 0.005 bar/LMH. Filtrate turbidity before reaching their maximum loading capacity remains equivalent for all filters around 5 NTU, excepted for PCF1 filter with a filtrate turbidity before breakthrough around 50 NTU. This higher filtrate turbidity can be explained due to the open nominal exclusion range of the PCF1 above all filters.

The combination PCF1+SCF1 showed promising results with representative CCF with a maximum loading capacity of 250 L/m² at a maximum pressure of 3 bar, as for manufacturer's recommendations. Nevertheless, the best depth filtration tested is the combination of PCF2+SCF2 with 522% (600 L/m²) of the loading capacity of the SF1 at a maximum pressure of 3 bar, as for manufacturer's recommendations. In general terms, the combination of two filters, for primary and secondary clarification was more efficient than only one filter such as SF1 or SF3. This combination allows to decrease the filtration surface and potentially the cost of the process.

### EXAMPLE 4: ASSESSMENT OF THE EFFECT OF A CENTRIFUGATION STEP PRIOR DEPTH FILTRATION

In order to assess the feasibility and the efficiency of centrifugation as primary clarification step, small-scale experiments were performed. These small scale experiments were intended to demonstrate the potential benefits of adding a centrifugation step prior to the depth filtration in order to facilitate the development of a clarification process and overcome process limitations.

### Characterization of the Required Centrifugation Parameters

A small-scale centrifugation study was performed in order to assess centrifugation parameters such as Relative Centrifugal Field (RCF) and time of centrifugation and their impact on the clarification metrics like turbidity, viability and cell lysis. This study also allowed to set-up correlations between centrifugation parameters and the clarification metrics.

Initial CCF from cell line B cultivated in fed-batch mode during 14 days in bioreactor with a turbidity of 944 NTU was treated by Eppendorf Centrifuge 5810 R at various RCF between 500 g and 3000 g for different times (1 min to 10 min).

As shown in FIG. 26 for 1 min of centrifugation step duration, the drop of the turbidity was directly linked with the centrifugation force. Indeed, at 500 g the turbidity decreases to 175 NTU (18.5%) compared to the initial value of 944 NTU (100%). In the same way centrifugation at 2000 G during 1 min was sufficient to decrease the initial turbidity to 45 NTU (5%) compared to initial value at 944 NTU (100%) by inducing the sedimentation of large particles responsible for the turbidity.

Centrifugation at 3000 G during 5 min was sufficient to decrease the turbidity of an initial CCF and this condition was selected when centrifugation was combined with depth filtration at small scale.

The previous CCF had an initial viability of 60% and a LDH concentration of 3042 U/L. After centrifugation, the cell pellet was suspended in PBS in order to conduct a viability measurement.

Centrifugation did not show any impact on the viability and LDH. According to these results, the centrifugation process does not introduce any cell breakage even at extreme centrifugation conditions like 3000 G during 10 min (described in FIG. 27 and FIG. 28).

Centrifugation at 3000 G during 5 min is enough to decrease the turbidity of the initial CCF to ensure a better clarification and this condition does not induce any cell lysis. This centrifugation set-up was then selected for the next experiment.

### Combination of Centrifugation and Depth Filtration: Worst-Case Clarification Study

The aim of this study is to assess the benefit of adding a centrifugation step before the depth filtration with a challenging CCF, as described in Table 17.

TABLE 17
Description of the CCF used in centrifugation and platform combination study

| | VCC | | Initial | | | |
|---|---|---|---|---|---|---|
| Cell | (10⁶ c/ | Viability | turbidity | Centrifugation | Centrifugation | Experimental |
| Line | mL) | (%) | (NTU) | time (min) | force (g) | conditions |
| A | 1.71 | 25 | 3380 | NA | NA | Thawed- CCF |
| A | NA | NA | 425 | 1 | 500 | Centrifuged CCF |
| A | NA | NA | 77.9 | 5 | 3000 | Centrifuged CCF |
| A | 12.15 | 83.4 | 1883 | NA | NA | Representative |
| | | | | | | CCF |

Initial thawed CCF showed, as expected, presents a large amount of dead cells and large debris that could likely lead to filter fouling as described in FIG. 29. After centrifugation treatment, the CCF showed a complete disappearance of large debris as only small vesicles can be seen. The CCF treated at 500 G, 1 min showed a turbidity decrease to 12% at 425 NTU of the initial value at 3380 (100%) NTU and low amount of cell and cell debris, as observed in FIG. 30. In the same way at 3000 G during 5 min the followed CCF showed no visible cell and debris but only small vesicles with a decrease to 2% of the turbidity at 77.9 NTU compared to the initial value at 3380 NTU (100%) (FIG. 31). The centrifugation process had a significant impact on turbidity and debris as shown in FIG. 29, FIG. 30 and FIG. 31.

In FIG. 32 and FIG. 33 at 50 L/m² the representative CCF had almost reached the limits for the clarification with a filtrate turbidity of 20 NTU and a pressure of 0.4 bar, whereas both centrifuged conditions showed the same behavior with a low turbidity at 10 NTU and a pressure at 0.2 bar.

Clarification conducted with centrifuged CCF in both conditions showed no sign of breakthrough with no turbidity and pressure increase over the time. Even gently centrifugation (1 min, 500 G) allowed to double the clarification load with a capacity of 100 L/m² under an acceptable pressure and turbidity of 0.2 bar and 5 NTU, respectively. Moreover, the maximum loading capacity was not reached in this case, due to a lack of initial CCF. It may be speculated that the SF1 filter with centrifuged CCF would handle a larger volume of CCF with only a slight increase of the pressure.

Compared with the representative CCF at 90 L/m², the centrifugation at 1 min, 500 g allowed a decrease to 4.3 NTU (11%) of the turbidity compared to 42 NTU (100%) with the untreated CCF and to 0.25 bar (31%) compared to 0.8 bar (100%) with the untreated CCF. This difference was even more significant compared with original thawed CCF with 220 NTU and 1.7 bar against 5 NTU and 0.2 bar at the same throughput.

This experiment demonstrated the benefits of a centrifugation step prior the depth filtration when depth filtration is facing limitations with challenging CCF.

### Combination of Centrifugation and Depth Filtration

The next experiment aimed to combine depth filters with a centrifugation step in order to compare their performance with and without centrifugation. A challenging CCF with the inputs below (Table 18) was used for this study. In this study both single step clarification filters such as SF2 and SF3 and secondary clarification filters such as SCF2 and SCF3 were tested.

TABLE 18
Description of the CCF

| | | | | | | Turbidity | |
|---|---|---|---|---|---|---|---|
| | Length of | TCC | VCC | | Initial | post- | |
| Cell | culture | (10⁶ c/ | (10⁶ c/ | Viability | turbidity | centrifugation | Experimental |
| Line | (days) | mL) | mL) | (%) | (NTU) | (NTU) | conditions |
| B | 14 | 12.1 | 7.89 | 65 | 2419 | — | Challenging |
| | | | | | | | bench scale |
| | | | | | | | CCF |
| B | 14 | NA | NA | NA | 2419 | 23.2 | Centrifuged |
| | | | | | | | CCF at |
| | | | | | | | 3000 G for |
| | | | | | | | 5 min |

Based on the previous evaluations, the following centrifugation parameters were selected: 3000 G during 5 min.

In this experiment, the clarification was stopped when the pressure reached 1.5 bar. As the used filters were not equipped with the same filter surface, the pressure was normalized by the liter/m²/hour (LMH) value to allow a comparison.

The filters could be merged in two groups with two specific behaviors as described in FIG. 34. Tight media filters (with narrow pores) such as SCF2 and SCF3 (see Table 3 for nominal exclusion range overview) show a rapid increase of their inlet pressure. Then, wide pore media filters such as SF2 and SF3 show a lower tendency to block. Among these two filters the SF3 showed a doubled higher loading capacity than the SF2 due to its synthetic composition. As the centrifugation step only removed large particles, it was expected to see that tight filters show a rapid tendency to block whereas wider pores allowed to filter a higher volume. For instance, the maximum loading capacity of the wide pore filter SF3 is 1200 L/m², compared to 250 L/m² for the SCF3, a filter with narrow pores.

The benefit of the centrifugation was demonstrated by comparing filter performance with untreated CCF and post centrifuged CCF.

The best centrifugation conditions were compared to conditions without centrifugation step (FIG. 35 and FIG. 36). At 50 L/m², the SF2 filter without centrifugation step exhibited a high resistance of 0.010 bar/LMH (i.e. pressure of 1 bar) correlated with a high turbidity of 50 NTU. SF3 filter without centrifugation step showed the same behavior with a less important resistance increase of 0.0015 bar/LMH (and 0.1 bar pressure) and no breakthrough (filtrate turbidity at 10 NTU). Breakthrough appeared at 100 L/m² with a filtrate turbidity increasing at 45 NTU and a filter resistance of 0.0025 bar/LMH (0.25 bar). Untreated initial CCF lead to a rapid filter fouling and breakthrough.

The centrifuged CCF showed no sign of breakthrough during the experiments with a turbidity which remained around 10 NTU for both SF2 and SF3 filters. At 50 L/m² both filters had the same low pressure near 0 bar/LMH but a different behavior was observed during the clarification. SF2 shows a faster clogging than SF3. Indeed, clarification with SF2 had to be stopped at 600 L/m² due to high pressure (1.3 bar) whereas SF3 was stopped at 1200 L/m² due to pressure increase (1.3 bar).

The addition of the centrifugation step improved the loading capacity of the SF2 filter by a factor of 17 and of the SF3 filter by a factor of 12 in comparison with experiments without a centrifugation step as described in Table 19 below.

This result demonstrated the positive effect of centrifugation, but also the importance of choice of the filter after the centrifugation step. This filter should have a suitable nominal exclusion range to remove mid and small sizes particles which will not be removed by the centrifugation itself.

TABLE 19
Summary of clarification results

| | | | | Pressure at | Turbidity at | |
|---|---|---|---|---|---|---|
| | | | Maximum | maximum | maximum | |
| | | Initial | loading | loading | loading | % |
| | | turbidity | capacity | capacity | capacity | Turbidity |
| CCF | Filter | (NTU) | (L/m²) | (bar) | (NTU) | reduction |
| Centrifuged CCF | SF2 | 2419 | 600 | 1.3 | 10 | 99.6 |
| Challenging bench | SF2 | 2419 | 35 | 1.2 | 20 | 99.2 |
| scale CCF | | | | | | |
| Centrifuged CCF | SF3 | 2419 | 1200 | 1.3 | 10 | 99.6 |
| Challenging bench | SF3 | 2419 | 100 | 0.3 | 15 | 99.4 |
| scale CCF | | | | | | |

In conclusion, the currently available results showed that the implementation of a centrifugation step would be an efficient way to improve the depth filter performances.

### EXAMPLE 5: ASSESSMENT OF THE EFFECT OF A FLOCCULATION STEP PRIOR DEPTH FILTRATION

Flocculation followed by depth filtration was investigated using challenging CCF by adding 0.03% (v/v) of pDADMAC to the cell culture described in Table 20.

TABLE 20
Description of the challenging CCF used in flocculation study

| | Length of | TCC | VCC | | Initial | Initial | |
|---|---|---|---|---|---|---|---|
| Cell | culture | (10⁶ c/ | (10⁶ c/ | Viability | turbidity | LDH | |
| Line | (days) | mL) | mL) | (%) | (NTU) | (U/L) | Experiment |
| B | 14 | 12.14 | 7.89 | 65 | 2410 | 3420 | Trial 3 |

Once the flocculation was performed, the broth was filtered through 23 cm² post-flocculation depth filters PFF1 and PFF2, specifically designed to fit the flocculation process (Table 21).

TABLE 21
Description of flocculation filters

| | | Filtra- | Maximum | | |
|---|---|---|---|---|---|
| | | tion | differential | Exclusion | |
| | | surface | pressure | range | General |
| Name | Media | (cm²) | (bar) | (μm) | comments |
| PFF1 | Polypropylene, | 23 | 2.1 | 0.6-19 | Post- |
| | cellulose, resin | | | | flocculation |
| | and filters aids | | | | filter |
| PFF2 | Polypropylene, | 23 | 2.1 | 0.6-40 | Post- |
| | cellulose, resin | | | | flocculation |
| | and filters aids | | | | filter |

The summary of the results is shown in Table 22. At the targeted throughput, the filters exhibited almost no difference in terms of pressure and turbidity as described in FIG. 37 and FIG. 38. The turbidity remained low around 3 NTU until 200 L/m² for PFF2 and 280 L/m² for PFF1. But the pressure increased faster in PFF2 with 0.60 bar at 280 L/m² instead of 0.40 bar for the PFF1.

Breakthrough occurred at 220 L/m² at a pressure of 0.2 bar for PFF2 whereas PFF1 exhibited a better loading capacity of 115% with a breakthrough starting at 290 L/m² under a 0.45 bar pressure.

The flocculation process allowed to largely increase the loading capacity compared to the SF1. As an example, 300 L/m² throughput was reached for the PFF1 and 280 L/m² for the PFF2, whereas the throughput for SF1 in the previous experiments was 115 L/m².

Both filters had to be stopped as the turbidity threshold was reached, and the pressure reached 0.75 bar. The maximum throughput was 275 L/m² for PFF2 and 312 L/m² for PFF1.

TABLE 22
Flocculation results summary

| | | | Pressure at | Turbidity at | |
|---|---|---|---|---|---|
| | | Maximum | maximum | maximum | |
| | Initial | loading | loading | loading | % |
| | turbidity | capacity | capacity | capacity | Turbidity |
| Filter | (NTU) | (L/m²) | (bar) | (NTU) | reduction |
| Floccu- | 2410 | 312 | 0.75 | 18 | 99.3 |
| lation + | | | | | |
| PFF1 | | | | | |
| Floccu- | 2410 | 275 | 0.75 | 20 | 99.2 |
| lation + | | | | | |
| PFF2 | | | | | |

### EXAMPLE 6: ASSESSMENT OF THE EFFECT OF AN AWS STEP PRIOR DEPTH FILTRATION

The impact of acoustic wave separation as primary clarification step on the subsequent depth filtration step was investigated according to the experimental conditions in Table 23.

TABLE 23
Description of the CCF used in AWS study

| | | Length of | TCC | VCC | | Initial | Turbidity |
|---|---|---|---|---|---|---|---|
| Experimental | Cell | culture | (10⁶ c/ | (10⁶ c/ | Viability | turbidity | post-AWS |
| conditions | Line | (days) | mL) | mL) | (%) | (NTU) | (NTU) |
| Initial CCF | B | 14 | 10.5 | 9.6 | 90.6 | 1770 | — |
| pre AWS | | | | | | | |
| CCF post | B | 14 | 4.68 | 4.16 | 88.9 | 1770 | 533 |
| AWS | | | | | | | |
| treatment | | | | | | | |

The comparison of the clarification performance (pressure) of SF1 and PCF1+SCF1 combination on untreated CCF and CCF treated with AWS, shown in FIG. 39, indicates that SF1 and PCF1+SCF1 with untreated CCF showed major pressure increase with 1.5 bar reached respectively at 89 L/m² and 108 L/m². The same filters with AWS treated CCF exhibited 2 fold increase of clarification performance with 188 L/m² reached at 1.35 bar for the SF1 and 200 L/m² reached at 1.5 bar for the PCF1+SCF1, considering this range of pressure threshold. Therefore, AWS pre-treatment allowed at least a doubling of the clarification performance (induced by the initial turbidity reduction of 60%).

The comparison of clarification performance (turbidity) of SF1 and PCF1+SCF1 combination on untreated CCF and CCF treated with AWS (FIG. 40) indicates that SF1 on untreated CCF showed an early breakthrough at 47 L/m² (turbidity going above 10 NTU). The same filter on AWS treated CCF exhibited a later breakthrough at a throughput of 113 L/m². No breakthrough was detected with PCF1+SCF1 combination either with or without AWS treatment. The turbidity and VCC reduction brought by the AWS was enough to avoid any breakthrough issue and the pressure was the only limiting factor in this case, considering the use of the best combination of filters PCF1+SCF1.

In conclusion, starting from a cell culture with densities <10×10⁶ cells/mL, AWS allows a 60% turbidity reduction. Additionally, AWS allows an improvement on the throughput by a factor 2 on the subsequent depth filtration.

TABLE 24
AWS results summary

| | | | Pressure at | Turbidity at | |
|---|---|---|---|---|---|
| | | Maximum | maximum | maximum | |
| | Initial | loading | loading | loading | % |
| | turbidity | capacity | capacity | capacity | Turbidity |
| Filter | (NTU) | (L/m²) | (bar) | (NTU) | reduction |
| SF1 | 1770 | 68 | 0.94 | 26 | 98.5 |
| AWS + | 553 | 113 | 0.83 | 50 | 90.6 |
| SF1 | | | | | |
| PCF1 + | 1770 | 138 | 2.34 | 3.15 | 99.8 |
| SCF1 | | | | | |
| AWS + | 553 | 225 | 1.72 | 10 | 98.1 |
| PCF1 + | | | | | |
| SCF1 | | | | | |

### EXAMPLE 7: SMALL SCALE AND BENCH SCALE CLARIFICATION OF CELL LINE C CELLS

Based on the results obtained in the previous experiments using cell lines A and B, further investigations have been carried out by testing SF1 filter and the most promising combinations of filters for the clarification of CFF obtained by cell line C. The experiments performed were done at small and bench scale according to Table 25.

TABLE 25
List of experiments and filter tested

| Experiment | Filters tested | Scale |
|---|---|---|
| Exp1 | SF1 | Bench (0.1 m²) |
| Exp2 | PCF2 + SCF2 | Small (0.0023 m² + 0.0023 m²) |
| | SF1 + SCF1 | Small (0.0022 m² + 0.0022 m²) |
| | PCF1 + SCF1 | Small (0.0022 m² + 0.0022 m²) |
| Exp3 | PCF2 + SCF2 | Small (0.0023 m² + 0.0023 m²) |
| | PCF1 + SCF1 | Small (0.0022 m² + 0.0022 m²) |
| | PCF1 + 2 × SCF1 | Small (0.0022 m² + 0.0044 m²) |
| Exp4 | 2 × PCF2 + SCF2 | Small (0.0046 m² + 0.0023 m²) |
| | PCF2 + SCF2 | Small (0.0023 m² + 0.0023 m²) |
| Exp5 | 2 × PCF2 + SCF2 | Small (0.0046 m² + 0.0023 m²) |
| | PCF2 + SCF2 | Small (0.0023 m² + 0.0023 m²) |
| Exp6 | 2 × PCF2 + SCF2 | Bench (0.027 m² + 0.014 m²) |
| Exp7 | PCF2 + SCF2 | Bench (0.027 m² + 0.027 m²) |
| Exp8 | 2 × PCF2 + SCF2 | Bench (0.027 m² + 0.014 m²) |
| Exp9 | PCF2 + SCF2 | Small (0.0046 m² + 0.0046 m²) |
| | 2 × PCF2 + SCF2 | Small (0.0046 m² + 0.0023 m²) |

The initial CCF characterization is described in Table 26. Key parameters are considered, such as the VCC, viability, turbidity, titer and LDH. To challenge the clarification, the process duration is extended with the aim of increasing the turbidity.

TABLE 26
Initial characterization of the CCF used

| | Culture | VCC | | Initial | Initial | Initial |
|---|---|---|---|---|---|---|
| Experi- | duration | (10⁶ c/ | Viability | turbidity | CEDEX | LDH |
| ment | (days) | mL) | (%) | (NTU) | titer (g/L) | (U/L) |
| Exp1 | 13 | 25.9 | 84.7 | 3820 | 6.75 | 3021 |
| Exp2 | 13 | 25.9 | 84.7 | 3820 | 6.75 | 3021 |
| Exp3 | 14 | 27.3 | 83.9 | 4908 | 7.71 | 2987 |
| Exp4 | 14 | 25.0 | 82.8 | 5076 | 7.70 | 2676 |
| Exp5 | 18 | 16.7 | 66.0 | 5670 | 8.46 | 3911 |
| Exp6 | 18 | 16.7 | 66.0 | 5670 | 8.46 | 3911 |
| Exp7 | 15 | 22.26 | 82.3 | 4032 | 7.011 | 2314 |
| Exp8 | 15 | 22.26 | 82.3 | 4032 | 7.011 | 2314 |
| Exp9 | 15 | 22.26 | 82.3 | 4032 | 7.011 | 2314 |

All small scale screening are done with the tri-headed pump FilterTec. In this experiment, the flow is fixed for the tested conditions. In case of a bench scale test, a calibrated pump is used depending on the tubing size need. All the calculations are made based on the current SUB platform process, which is a targeted ratio of 50 L/m2, at 70 Liter/square meter/hour (LMH). Filters are rinsed and equilibrated following the supplier's recommendation. The clarification process was monitored over the time by following some key indicator parameters, such as pressure of the inlet filter and before the secondary filter when applicable, turbidity of the CCCF, product titer, LDH, weight (to calculate the throughput in L/m 2).

### Characterization of SF1

First Exp1 as described in Table 25 and Table 26 was performed, the pressure trends obtained are illustrated in FIG. 41. The maximum pressure reached was 0.4 bar at the end of the clarification, in the inlet of the SF1. It remained below the 1 bar limit so inlet pressure was not a limiting factor. Pressure in bioburden reduction filter remained below 0.1 bar. As a conclusion, all the available material has been clarified through the filter without a major pressure increase and the filter maximum capacity (blockage) was not reached.

The turbidity trends are shown in FIG. 42. The two turbidity trends follows the same pattern. When the turbidity post SF1 increases, the related turbidity in outlet of the bioburden reduction filter also increases with an offset of 2 NTU. The turbidity rapidly increased over the throughput to reach a maximum of 10 NTU at 28 L/m². The corresponding turbidity in the bioburden reduction filter was 8.5

NTU. After this measurement, the filters started to be flushed with PBS, because of a lack of initial material, as a consequence, the turbidity started to decrease. To conclude, due to the limited amount of material (3.2 L), the maximum turbidity was not higher than the 10 NTU threshold. Nevertheless for a theoretical volume of 5 L turbidity would exceed the threshold. The maximum throughput obtained in this experiment is below the expected target of 50 L/m².

LDH and titer were also monitored. LDH enzyme is an intracellular protein. Monitoring its level in the supernatant is indirectly reporting the clarification efficacy by cell lysis follow-up. Product titer is also a key parameter to define the step yield and will show if the filter could retain the product of interest (by interactions with the filter). The two trends are shown in FIG. 43. LDH was constantly increasing when the clarification started, and it overcame CCF initial value of 3021 U/L at a throughput of 19 L/m². As previously described, the PBS flush started at a throughput of 28 L/m2, explaining the LDH decrease. The final value in CCCF is 1860 U/L. As the LDH level is passing the initial measurement, a cell lysis started to happen at low throughput (19 L/m2). Titer trend follows the same pattern but never reaches the value of the CCF (6.75 g/L).

Clarification yields were assessed and are shown in Table 27.

TABLE 27
Yield and volume comparison pre- and post-clarification

| | Pre-clarification | Post-clarification |
|---|---|---|
| HPLC-PA titer (g/L) | 6.63 | 2.45 |
| Volume (L) | 3.2 | 5.4 |

After PBS dilution, the final titer was reduced from 6.63 g/L to 2.45 g/L. PBS flush was stopped because of the bag volume limitation (5 L). The dilution factor reached is 68%. This high dilution is explained by the high dead volume of the filter (2.3 L). Even if the dilution ratio is high, the calculated yield is only of 63%, which is low for a clarification step (usually >95%). Two explanations are possible. Some product could still remain in the filter, meaning that the flush was not sufficient, or product is adsorbed to the membrane. The two hypothesis are verified by monitoring some metabolites concentrations to assess the recovery in the broth as shown in Table 28.

The recovery is calculated as follows:

$$Recovery ( \% ) = \frac{( CCCF [ metabolite ] \times CCCF volume )}{CCF [ metabolite ] \times CCF volume} \times 100$$

TABLE 28
Monitoring of key metabolites

| Process | Volume | Lactate | Glutamine | Glutamate | Ammonia | HPLC-PA | CEDEX |
|---|---|---|---|---|---|---|---|
| step | (L) | (g/L) | (mM) | (mM) | (mM) | titer (g/L) | titer (g/L) |
| CCF (pre | 3.2 | 3.95 | 8.75 | 0.58 | 5.88 | 6.63 | 6.75 |
| clarification) | | | | | | | |
| CCCF (post | 5.4 | 1.58 | 3.21 | 0.23 | 2.17 | 2.452 | 2.512 |
| clarification) | | | | | | | |
| Recovery | / | 68 | 62 | 67 | 62 | 62 | 63 |
| (%) | | | | | | | |

The recovery is similar between key metabolites parameters and titer (average of 64%). It means that the product is not adsorbed on the filter, instead it shows that the filter was not flushed enough to recover all the product. This confirmed that the high dead volume of the filter (2.3 L) is a limiting factor when a low volume of CCF is available.

### Small Scale Assessment of Filter Combinations—Part 1

Next experiment Exp2 was performed, testing combination of filters at small scale according to Table 25 and Table 26.

The pressure of small scale filters is in shown in FIG. 44. The SF1+SCF1 combination is the first filter to reach the pressure limit, at a throughput of 80 L/m². At 1 bar, the throughput reached is 50 L/m². The addition of the SCF1 filter after SF1 allows to slightly increase the platform capacity when compared to the results obtained previously with the SF1 filter only (28 L/m²), nevertheless, clogging of the filters still occurs quickly. PCF1+SCF1 combination is the second to reach the 3 bar limit, with a maximum throughput of 120 L/m² reached. This throughput is decreased to 95 L/m² when limited at 1 bar. PCF2+SCF2 combination reaches 3 bar at a throughput of 230 L/m². At 1 bar, the throughput obtained is 158 L/m².

The associated turbidities are plotted in FIG. 45. In all the three combinations, the turbidity always remained below the 10 NTU threshold. It means that the secondary filter, which is a tight filter, is retaining all the particles and limiting the turbidity increase. The trend observed with the PCF2+SCF2 filters combination shows a lower turbidity compared to PCF1+SCF1 filters (<5 NTU), which could be explained by a slower pressure increase over the throughput. As a conclusion, turbidity is not the limiting factor in this experiment. The secondary filter is never facing breakthrough and the maximum tolerated pressure will be the stopper of the experiment.

LDH and titer are analyzed, the final level of LDH and product titer are shown in Table 29.

TABLE 29
Final level of LDH and product titer

| | Initial | PCF2 + | SF1 + | PCF1 + |
|---|---|---|---|---|
| | Material | SCF2 | SCF1 | SCF1 |
| CEDEX titer (g/L) | 6.75 | 6.05 | 5.81 | 5.97 |
| LDH (U/L) | 3021 | 4607 | 5816 | 5308 |

Final titers are comparable between the 3 filters, and slightly lower than the initial titer. This could be explained by the residual dilution of the broth with the flush and equilibration buffers. LDH level is higher than the initial one in all the three filters, but is slightly lower in PCF2+SCF2 combination. Cell lysis phenomenon occurs in all the cases.

The product quality of the antibody in the CCCF has been also assessed so as to make sure that the filtration does not have an impact on the antibody. As shown in Table 30, no differences are observed on fragments (Fgmts), aggregates (Agg) and main species (Main) on SE-HPLC method, demonstrating that none of the tested filters have an impact on the purity of the product.

TABLE 30
Product purity by SE-HPLC

| | Initial | PCF2 + | SF1 + | PCF1 + |
|---|---|---|---|---|
| | Material | SCF2 | SCF1 | SCF1 |
| PA SEC - Agg | 0.7 | 0.85 | 0.92 | 0.92 |
| PA SEC - Main | 99.1 | 99 | 98.9 | 98.9 |
| PA SEC - Fgmts | 0.18 | 0.19 | 0.18 | 0.16 |

A Further purity analysis was performed by Capillary gel electrophoresis (CGE), the results are shown in Table 31 and Table 32.

TABLE 31
Purity by non-reduced CGE

| | Initial | PCF2 + | SF1 + | PCF1 + |
|---|---|---|---|---|
| | Material | SCF2 | SCF1 | SCF1 |
| PA CGE NR - LC | 1.9 | 3.2 | 3.5 | 3.2 |
| PA CGE NR - | 0.5 | 0.8 | 1 | 0.5 |
| Fragment 75 kDa | | | | |
| PA CGE NR - | 1.5 | 1.6 | 1.2 | 1 |
| Fragment 100 kDa | | | | |
| PA CGE NR - | 5.9 | 9.6 | 10 | 9.4 |
| Fragment 125 kDa | | | | |
| PA CGE NR - IgG | 89.9 | 84.5 | 84 | 85.5 |

TABLE 32
Purity by reduced CGE

| | Initial | PCF2 + | SF1 + | PCF1 + |
|---|---|---|---|---|
| | Material | SCF2 | SCF1 | SCF1 |
| PA CGE R - LC | 34.4 | 34.4 | 35.2 | 33.8 |
| PA CGE R - NG | 0.7 | 0.6 | 0.9 | 0.9 |
| PA CGE NR - HC | 64.9 | 65.1 | 63.9 | 65.4 |

In non-reduced CGE, no difference were observed between the three filters combinations. A slight difference is observed when compared to the initial CCF, where a 5% shift is observed between intact IgG and 125 kDa fragment. This difference could be explained by the fact that the broth was kept open over the clarification trials and the product could slightly be fragmented during this incubation period. When comparing the reduced profiles, no difference were detected.

Charge profile is also characterized by iCE3, as shown in Table 33.

TABLE 33
Charge profile monitoring by iCE3

| | Initial | PCF2 + | SF1 + | PCF1 + |
|---|---|---|---|---|
| | Material | SCF2 | SCF1 | SCF1 |
| PA iCE3 - Sum Acidics | 42.7 | 45 | 45.4 | 45.3 |
| PA iCE3 - Main | 49.7 | 47.7 | 47.2 | 47.2 |
| PA iCE3 - Sum Basics | 7.7 | 7.2 | 7.4 | 7.5 |

No difference was observed between the three filters tested in terms of acidics, main and basics species. A slight difference with the initial material was observed, with a 2% shift from main to acidics. It could also be explained by the fact that the broth was kept open at room temperature during the trials.

Finally, the carbohydrates profile was monitored by HILIC-UPLC, as illustrated in Table 34.

TABLE 34
Carbohydrates profile by HILIC-UPLC

| | Initial | PCF2 + | SF1 + | PCF1 + |
|---|---|---|---|---|
| | Material | SCF2 | SCF1 | SCF1 |
| PA Glycans - Man5 | 3.39 | 3.32 | 3.15 | 3.34 |
| PA Glycans - G0F | 59.4 | 59.5 | 59.3 | 59.6 |
| PA Glycans - G1F | 13.9 | 14.1 | 14.4 | 14.1 |
| PA Glycans - G1′F | 5.42 | 5.45 | 5.64 | 5.49 |
| PA Glycans - G2F | 1.73 | 1.7 | 1.86 | 1.84 |

No difference was observed in all the main carbohydrates species, confirming that the product quality is not impacted by the filters tested.

### Small Scale Assessment of Filter Combinations—Part 2

Experiment Exp3 was then performed according to Table 25 and Table 26 to further test the combinations of filters PCF1+SCF1 and PCF2+SCF2 as well as the combination of PCF1 followed by two SCF1 (PCF1+2×SCF1) when a 14 days old fed-batch broth is used with higher turbidity than the one of Exp2.

The pressure plots are in FIG. 46. PCF1+SCF1 combination is the first one to reach the limit of 3 bars, at a throughput of 80 L/m². It is followed by the PCF1+2×SCF1 combination with a maximum throughput of 114 L/m² reached. At 1 bar, the two filters are exhibiting a similar throughput of 60 L/m². The benefit of having two times more surface of the second filter is clear when the 1 bar pressure is reached. PCF2+SCF2 shows better results, with a throughput of 170 L/m² reached at 3 bars. At 1 bar, the throughput obtained is 110 L/m², which is close to two times the capacity allowed by the other alternatives. In summary, PCF1+SCF1 demonstrated faster clogging issue but the platform performances of 50 L/m² are reached. Adding twice more SCF1 allowed to reach better performances (120 L/m²), showing that SCF1 was limiting the capacity, but only after 1 bar of pressure in the system. PCF2+SCF2 remained the best combination, with a throughput of the 170 L/m² reached.

The associated turbidities are in FIG. 47. In all the three combinations, the turbidity always remained below the 10 NTU threshold. It means that the secondary filter, which is a tight filter, is retaining all the particles and limiting the turbidity increase. The trend observed with PCF2+SCF2 filters shows a lower turbidity compared to the other filters, which could be explained by a slower pressure increase over the throughput. As a conclusion, turbidity was not the limiting factor in this experiment. The secondary filter is never facing breakthrough and the maximum tolerated pressure will be the stopper of the experiment.

The LDH kinetic is then assessed, the results are shown in FIG. 48. LDH rapidly increases in all the filters and crosses the initial value of 3000 NTU, meaning that the enzyme is released in the supernatant and a cell lysis is happening. Clarification of this CCF shows a LDH release over the process, correlated with the pressure applied to filters.

### Small Scale Assessment of Filter Combinations—Part 3

The aim of the study was to confirm the results obtained with previous fed-batches, cultivated for 14 days, but also to challenge the clarification step by extending the cell culture process duration, and hence, increase the turbidity of the initial broth. The combination of filters PCF2+SCF2 and the combination of two PCF2 filters followed by SCF2 (2×PCF2+SCF2) were tested in the conditions of experiments Exp2 to Exp5 according to Table 25 and Table 26.

The pressure plots are in FIG. 49. PCF2+SCF2 ratio 1:1 pressure shows good correlation with the initial broth state, except for Exp3 where the filters behave worse than expected. It could be explained by filters variability at small scale. Indeed, a small air bubble trapped in the filter would reduce the filtration surface and hence affects the performances. PCF2+SCF2 ratio 2:1 were not challenged till the maximum capacity because of a low flow applied. In fact, since ratio 1:1 and 2:1 are run in parallel with the same tri-headed pump, it is not possible to increase the flow for the ratio 2:1. As the surface is doubled, it means that the volume to be passed through the filter will be twice more and the time twice longer. As a conclusion of this comparison, a throughput of 197 L/m² is observed at 3 bar with the most challenging CCF (Exp5) in ratio 1:1, and decreasing to 110 L/m² at 1 bar. Based on this result, the throughput obtained at 1 bar is two times the targeted expectation meaning that it will be working with this worst CCF. In ratio 2:1, the maximum throughput obtained is around 160 L/m² before trial stop. This experiment showed that it is not possible to challenge the ratio 2:1 at small scale using this LMH (and flow rate).

The related turbidities trends are shown in FIG. 50. No major turbidity differences are observed between ratio 1:1 and 2:1, except for Exp4, where a breakthrough happened at the end of the trial in ratio 1:1, with a turbidity of 65 NTU. PCF2+SCF2 turbidity with the worst CCF increased close to 15 NTU. An increase of small particles is starting maybe because of a cell lysis phenomenon. As a conclusion, turbidity is not the limiting factor in this experiment.

The cell lysis rate is also monitored (FIG. 51) by following the LDH levels over the throughput. The quantity of enzyme is crossing the initial level quickly (between 3000 and 4000 U/L), to reach around 2 to 2.5 times the initial amount at the end of the trial. In Exp4, the sudden increase in ratio 1:1 is correlated with pressure (>1 bar) and bottle change. In summary, even if the turbidity level remains acceptable, some cell lysis is happening.

Process Scalability from Small to Bench Scale with a Worst Case CCF

Along with the small scale experiment Exp5, a bench scale trial was performed with the same CCF and with 2×PCF2+SCF2 (Exp6), according to Table 25 and Table 26.

Flow rate and LMH verification is the first step of the data analysis, especially to consider a scale-up. Even if the pump is calibrated to match the desired flow rate, cells going through the tubes will enhance slight modifications of the flow. This is monitored in FIG. 52 for the bench scale trial. The real flow measured over the time is 28.9 mL/min, instead of the theoretical flow of 32 mL/min, calibrated with water. It results in a constant LMH of 64.2 instead of the targeted 70 LMH.

Pressure trends associated to the filters are shown in FIG. 53. Pressure is monitored in inlet of the PCF2 filter and between the two filters. If the pressure trends are parallel, it means that both filters are efficiently working. This is the case at small scale (Exp5) but a higher pressure increase in the bench scale trial (Exp6) is detected. An inflexion in the primary pressure is starting at 60 L/m² which has a direct impact on SCF2 pressure, where the inflexion is happening slightly later at 70 L/m². A different behavior is observed at bench scale, where a breakthrough is happening in the primary filter, which is not detected at small scale. Performances are affected, with 2 times less capacity at bench scale considering the 1 bar threshold (136 L/m² vs. 67 L/m²).

In parallel with the pressure trends, the turbidity is monitored as shown in FIG. 54. No major turbidity difference is observed till a throughput of 65 L/m² is reached (1 bar) between small scale (Exp5) and bench scale (Exp6). Turbidity increased post PCF2 after 1 bar in bench scale trial which correlates with an increase in SCF2 turbidity later on. One third of the initial turbidity is going through the PCF2 filter confirming that a breakthrough is happening. As the load in high for the secondary SCF2 filter, a breakthrough is also happening at the end of the trial. It is not the case at small scale, where the turbidity always remained low (<13 NTU).

Vicell pictures were taken on key samples to monitor the state of the breakthrough. The pictures are shown in FIG. 55. The sampling done at 1 bar in both filters (A and B pictures) indicates that the breakthrough starts in the PCF2 filter, without affecting the SCF2 performance. The following sampling post SCF2 (C) showed that the dynamic breakthrough also occurs for the SCF2 filter.

The cell lysis level, monitored by LDH assay is shown in FIG. 56. The LDH levels are comparable in both experiments till a throughput of 70 L/m² is reached. After this threshold, the LDH in bench scale trial is increasing rapidly, correlated with the previous results where a breakthrough is happening. The breakthrough is forcing the cells to go through the filter, which could induce cells bursting and hence, LDH release. As a conclusion of this comparison, the bench scale trial is showing lower performances compared to the small scale results. Which is due to the fact that twice more volume was passed through the same surface area at bench scale.

### Process Scalability to Bench Scale

The combination of filters PCF2+SCF2 and the combination of two PCF2 filters followed by SCF2 (2×PCF2+SCF2) was tested at bench scale with a best case CCF. As previously described, the flow rate and LMH verification is the first step of the data analysis, especially to consider a scale-up. This is monitored in FIG. 57 for the bench scale trials Exp7 and Exp8 (see able 25 and Table 26 for experiments conditions). The real flow measured over the time is respectively of 26.9 mL/min and 26.0 mL/min in both experiments, which results in a constant LMH of 59.8 and 57.8 respectively. As a reminder, the targeted LMH is 70. In all the small scale experiments, the theoretical LMH was matched, with calculated values of 73.2 LMH in ratio 1:1 and 71.7 LMH in ratio 2:1 (graphs not shown).

The associated pressure trends in both bench and small scale experiments are compared in FIG. 58. For PCF2+SCF2, it is visible that the pressure trend in primary filter is similar between small scale and bench scale runs, until a throughput of 44 L/m² is reached. After this threshold, the pressure in bench scale trial is increasing suddenly to reach 1.5 bar at 96 L/m². The inlet pressure of the secondary filter at bench scale also increased proportionally to the increase in primary filter pressure, to reach 1.15 bar at the end of the trial. Small scale pressure increase remained slow and the trial ended at a throughput of 72 L/m² because of a lack of initial material. For 2×PCF2+SCF2, the pressure increase between both scales remained similar till a throughput of 72 L/m² is reached, where the small scale trial was stopped for the same reason than the one mentioned above. The final pressure reached in the primary bench scale filter is 0.7 bar, below the threshold limit of 1 bar. The pressure in the secondary filter remained flat till a throughput of 89 L/m² is reached. After this capacity, pressure started to suddenly increase, probably showing the start of a clogging of the filter. In none of the trials detailed above, the pressure was a limiting factor. In the ratio 1:1 at bench scale, the pressure went above 1 bar between the two last samplings, with mean that the maximum trial capacity is reached, given the set pressure limit of 1 bar. Nevertheless it would still be possible to go higher than 1 bar if turbidity is still fine.

The turbidity associated to each trials is shown in FIG. 59. The CCCF turbidity (post clarification) always remained below the 10 NTU threshold in all the trials. It means that the turbidity was not a limiting factor in the experiments. In the ratio 1:1 trial, a breakthrough started to happen at a throughput of 54 L/m². After this capacity, the turbidity gradually increased to reach 2400 NTU at a throughput of 89 L/m². It then started to decrease because of the PBS flush initiated at a throughput of 76 L/m². This PBS flush is performed with the aim to flush the high dead volume of the filters and recover a maximum of product. The experiment was stopped when the product started to be diluted. In the ratio 2:1, a similar behavior is observed, with a turbidity increase when a capacity of 67 L/m² is reached. The maximum turbidity value reached is 1800 NTU at a throughput of 89 L/m². A slight impact is detected in the CCCF turbidity, because of a lower surface of secondary filter compared to the ratio 1:1 (0.027+0.027 in ratio 1:1, 0.027+0.014 in ratio 2:1) but the measured value remained under the threshold limit.

### EXAMPLE 8: PILOT SCALE CLARIFICATION OF CELL LINE C CELLS

### Experimental Setup

Following the experiments of Example 7 a 50 L pilot process was run. The CCF generated in 50 L SUB is described and compared with the bench scale broth in the Table 35.

TABLE 35
Initial CCF description at bench and pilot scales

| | | CCF | | CCF | CCF | |
|---|---|---|---|---|---|---|
| | Culture | VCC | CCF | initial | initial | CCF |
| Experiment | duration | (10⁶ c/ | Viability | turbidity | LDH | titer |
| ID | (days) | mL) | (%) | (NTU) | (U/L) | (g/L) |
| Exp8 | 15 | 22.3 | 82.3 | 4032 | 2314 | 7.011 |
| Exp10 | 14 | 21.4 | 88.4 | 4165 | 2078 | 7.713 |

After 14 days of culture, the initial turbidity was similar between both scales. The viability and the titer were slightly higher at pilot, but the overall broth comparison remained similar. At pilot, a safety margin on filters surface is added. The clarification has then started as described in the previous sections and the first steps were the WFI and PBS flushes. The dead volume characterization is then the first step where a monitoring is required.

### Clarification

### Flow Monitoring

The first step to verify that the clarification is well performing is to measure the real flow of the step. In theory, based on calculations, the flow should be of 0.898 L/min. The tolerated margin is +/−5% as considered as an acceptable clarification variability. The experimental flow is detailed in FIG. 60. The measured flow is linear, with a high RSquare Adj of 0.998. The LMH is then constant as targeted. The measured flow over the clarification is 0.938 L/min, which is within the +/−5% variability accepted.

### Throughput Characterization

In order to verify that the filters are not clogging over the time, the throughput (L/m²) was plotted over the time (FIG. 61). No throughput decrease is observed over the time, meaning that there is not filter clogging.

### Pressure Monitoring

Next pressure was monitored. As previously described, three pressure sensors are added into the set-up to allow the detection of any pressure increase in the three filters of the process, namely the primary clarification filter PCF2, the secondary clarification filter SCF2 and Sartopore 2 bioburden reduction filter. The pressure trends are shown in FIG. 62. The PCF2 inlet filter pressure slightly increases over the throughput but remained below 0.326 bar (FIG. 62, top panel). The inlet pressure of the secondary filter (FIG. 62, middle panel) and of the bioburden filter (FIG. 62, bottom panel) remained also low, of 0.203 for SCF2 and 0.100 bar for Sartopore 2 sterile filter at the maximum. No pressure increase was globally observed in this experiment. It showed that the sizing was well estimated and the safety factor is allowing this pressure control.

### Turbidity Monitoring

As per previous developments, the turbidity post depth filtration should be between 10 and 20 NTU as a maximum to avoid clogging issues of the 0.22 μm filter. Plots are shown in FIG. 63. Turbidity slightly increased post PCF2 filter, as this filter is removing the cells. It reached 22 NTU which is not critical compared to the values observed at bench scale without safety factor (1800 NTU, not shown). CCCF turbidity always remained low (<4 NTU) and comparable to what was observed in bench scale (<8 NTU). As a conclusion, no turbidity increase was detected in the CCCF. The safety margin is also allowing to control the turbidity increase post PCF2 primary filter (which was way higher in bench scale trial, without any effect on SCF2 filter).

### Cell Lysis Evaluation by LDH Measurement

LDH trends measured by the CEDEX metabolite analyzer are shown in FIG. 64. Cell lysis level by LDH increased till around 2400 U/L in both filters, which is close to the initial value (2078 U/L). The delay in the throughput to reach the maximum value is linked to the dead volume of the filters. At the end of the experiment, the LDH is decreased because of the PBS flush, which is diluting the broth. Therefore, no cell lysis is induced by the clarification in this experiment as the LDH level is not increasing highly above the initial value.

### Product Titer Evaluation

The product titer over the clarification is shown in FIG. 65. Product recovery is similar through both filters and decreased post PBS connection to flush the dead volume of the filters. At the end of the clarification, still 2 g/L of product was in the filters.

### Product Quality Analysis Pre/Post Clarification

In order to assess the clarification impact on product quality, the antibody is analyzed for purity by SE-HPLC (Table 36) and both non-reduced (Table 37) and reduced CGE (Table 38). It is also analyzed for charge by cIEF (Table 39) and for carbohydrate by HILIC-UPLC (Table 40).

TABLE 36
SE-HPLC product profile before and after clarification

| | Exp10 before | Exp10 after |
|---|---|---|
| | clarification | clarification |
| PA SEC - Agg | 1.08 | 0.56 |
| PA SEC - Main | 98.8 | 98.3 |
| PA SEC - Fgmts | 0.11 | 1.12 |

As shown in Table 36, the main % is identical between the two conditions, before and after centrifugation. A slight shift (0.5%) from aggregates to fragments is observed over the clarification. The clarification process increased the fragment levels by fragmenting the aggregates, this could be due to the pressure in the clarification system, induced by the pump flow speed.

TABLE 37
Non-reduced (NR) CGE product profile
before and after clarification

| | Exp10 before | Exp10 after |
|---|---|---|
| | clarification | clarification |
| PA CGE NR - LC | 3.9 | 3.5 |
| PA CGE NR - | 1 | 0.7 |
| Fragment 75 kDa | | |
| PA CGE NR - | 1.4 | 1.2 |
| Fragment 100 kDa | | |
| PA CGE NR - | 12 | 11.6 |
| Fragment 125 kDa | | |
| PA CGE NR - IgG′ | 1 | 0.9 |
| PA CGE NR - IgG | 80.4 | 82 |

As shown in Table 37, the overall CGE profile is similar before and after the clarification. This process step is not inducing any remarkable fragmentation of the main product. The same conclusion is observed in reduced CGE (Table 38), where no difference is observed.

TABLE 38
Reduced CGE product profile before and after clarification

| | Exp10 before | Exp10 after |
|---|---|---|
| | clarification | clarification |
| PA CGE - LC | 31.5 | 31.4 |
| PA CGE - NG | 0.8 | 0.7 |
| PA CGE - HC | 67.7 | 67.9 |

The charge profile also did not affected by the clarification, in fact no difference is observed between acidics, main and basics species before and after clarification (Table 39).

TABLE 39
Charge variation product profile measured
by cIEF before and after clarification

| | Exp10 before | Exp10 after |
|---|---|---|
| | clarification | clarification |
| PA cIEF - Sum Acidics | 56.4 | 55.5 |
| PA cIEF - Main | 38.3 | 39.2 |
| PA cIEF - Sum Basics | 5.3 | 5.3 |

The overall carbohydrate profile is also similar before and after the clarification as shown in Table 40.

TABLE 40
Carbohydrate product profile before and after clarification

| | Exp10 before | Exp10 after |
|---|---|---|
| | clarification | clarification |
| PA Glycans - G0 | 7.43 | 7.33 |
| PA Glycans - Man5 | 2.38 | 2.44 |
| PA Glycans - G0F | 57.6 | 57.3 |
| PA Glycans - G1 | 1.93 | 1.94 |
| PA Glycans - G1′ | 0.68 | 0.69 |
| PA Glycans - G1′F | 5.68 | 5.76 |
| PA Glycans - G2F | 2.12 | 2.14 |

Based on these data, no major differences were observed in the product quality before and after the clarification step.

1. A method for clarifying a cell culture including a biomolecule of interest and having a turbidity between about 1000 NTU and about 6000 NTU comprising (a) a primary clarification step which removes cell culture material of size equal to or greater than about 0.2 μm and (b) a secondary clarification step comprising filtration which removes cell culture material of size equal to or less than 4 μm, wherein said secondary clarification step has a maximum throughput equal to or greater than about 80 L/m² and wherein said primary and secondary clarification steps lead to a turbidity reduction equal to or greater than about 90%.

2. (canceled)

3. The method of claim 1, wherein said primary clarification is depth filtration performed by a first depth filter with exclusion range comprised between about 0.25 μm and about 30 μm.

4. The method of claim 3, wherein said first depth filter has an exclusion range comprised between about 5 μm and about 30 μm, or between about 0.5 μm and about 10 μm.

5.-6. (canceled)

7. The method of claim 3 wherein said secondary clarification is performed by a second depth filter with an exclusion range equal to or less than about 3.5 μm.

8. The method of claim 3, wherein when the cell culture has a turbidity less than about 3000 NTU, the maximum throughput is equal to or greater than about 250 L/m² and the turbidity reduction is equal to or greater than about 99%.

9. The method of claim 4, wherein when the cell culture has a turbidity equal to or greater than about 3000 NTU, the maximum throughput is equal to or greater than about 80 L/m² and the turbidity reduction is equal to or greater than about 99%.

10. The method of claim 1, wherein when the turbidity of said cell culture is less than 3000 NTU, said primary clarification step and said secondary clarification step are performed by a single filter with an exclusion range comprised between about 1 μm and about 20 said maximum throughput is equal to or greater than 110 L/m² and said turbidity reduction is equal to or greater than about 98%.

11. The method of claim 1 wherein said primary clarification is preceded by a cell culture pretreatment step of acoustic wave separation.

12. The method of claim 1 further comprising (c) a bioburden reduction step performed by one or more sterile filters with exclusion range equal to or less than 0.5 μm to obtain a clarified cell culture comprising said biomolecule of interest.

13. The method of claim 12 comprising (d) a further filtration step performed by a membrane absorber with exclusion range equal to less than 0.2 μm.

14. The method of claim 13 further comprising (e) a step of subjecting said clarified cell culture to one or more steps of purification of said biomolecule of interest.

15. The method of claim 1 wherein the cell culture comprises mammalian cells.

16. The method of claim 1 wherein said biomolecule of interest is an antibody or an antibody fragment thereof.

17. A cell culture subjected to the method of claim 1.

18. A process of production of a drug substance comprising the steps of:
(i) seeding cells expressing a biomolecule of interest in a cell culture medium;
(ii) culturing said cells for a period between 10 and 18 days, preferably for 14 days;
(iii) subjecting the obtained cell culture to the method according to claim 14 to purify the biomolecule of interest; and
(iv) adding excipients to the purified biomolecule of interest.
